(19) United States
(12) Patent Application Publication
(10) Pub. No.: US 2003/0027331 A1
(43) Pub. Date: Feb. 6, 2003

(54) Isolated homozygous stem cells, differentiated cells derived therefrom, and materials and methods for making and using same

(76) Inventors: Wen Liang Yan, Potomac, MD (US); Steve Chien-Wen Huang, Germantown, MD (US); Minh-Thanh Nguyen, Rockville, MD (US); Hua Lin, N. Potomac, MD (US); Lei Jingqi, Gaithersburg, MD (US); Ruchi Khanna, Germantown, MD (US)

Correspondence Address: Shanks & Herbert, TransPotomac Plaza, Suite 306, 1033 N. Fairfax Street, Alexandria, VA, 22314, US

(21) Appl. No.: 10/179,959

(22) Filed: Jun. 26, 2002

Related U.S. Application Data
(63) Continuation-in-part of application No. 09/997,240, filed on Nov. 30, 2001.
(60) Provisional application No. 60/253,943, filed on Nov. 30, 2000.

(51) Int. Cl. C12N 5/08

(52) U.S. Cl. 435/366

(57) ABSTRACT

The present invention discloses and describes pluripotent homozygous stem (HS) cells, and methods and materials for making same. The present invention also provides methods for differentiation of HS cells into progenitor (multipotent) cells or other desired cells, groups of cells or tissues. Further, the applications of the HS cells disclosed herein, include (but are not limited to) the diagnosis and treatment of various diseases (for example, genetic diseases, neurodegenerative diseases, endocrine-related disorders and cancer), traumatic injuries, cosmetic or therapeutic transplantation, gene therapy and cell replacement therapy.

The present application is a continuation in part of U.S. application Ser. No. 09/997,240, filed, Nov. 30, 2001, which claims priority to Provisinal U.S. Application Serial No. 60/253,943 filed on Nov. 30, 2001, the disclosure of which are all incorporated by reference herein in their entireties.

### I. FIELD OF THE INVENTION

The present invention discloses pluripotent homozygous stem (HS) cells, and methods and materials for making same. The invention also provides methods for differentiation of HS cells into progenitor cells or other desired cells, groups of cells or tissues. Further, HS cells disclosed herein may be used for the diagnosis and treatment of various diseases, such as genetic diseases, neurodegenerative diseases, endocrine-related disorders and cancer, traumatic injuries, cosmetic and therapeutic transplantation, and gene therapy and cell replacement therapy.

### II. BACKGROUND OF THE INVENTION AND DESCRIPTION OF RELATED ART

In 1981, Evans and Kaufman described the technique for isolating embryonic stem (ES) cell lines from mouse blastocysts. Establishment in Culture of Pluripotent Cells from Mouse Embryos,” Nature 292:154-6 (1981). In this procedure, the inner cell mass (ICM) was used to give rise to a cell line that remained undifferentiated and pluripotent, i.e., the cells had the capacity to develop into any cell type. ES cell lines were subsequently produced in other animal models including chicken (Pain et al, Development 122:2339-48 (1996)), hamster (Doetschmann et al., Dev. Biol. 127:224-7 (1988)), swine (Wheeler et al., Reprod. Fertil. Dev. 6:563-8 (1994)), marmoset (Thompson et al., Biol. Reprod. 55:254-9 (1996)), and rhesus monkey (Thompson et al., Proc. Natl. Acad. Sci. USA 92:7844-8 (1995)).

Saito et al., Roux's Arch. Dev. Biol., 201:134-141 (1992) reported bovine embryonic stem cell-like cell lines, which survived three passages, but were lost after the fourth passage. Further, Handyside et al., Roux's Arch. Dev. Biol., 196:185-190 (1987) disclosed culturing of immunosurgically isolated inner cell masses of sheep embryos under conditions which allowed for the isolation of mouse ES cell lines derived from mouse inner cell masses (“ICM”). It was further reported that under such conditions sheep ICMs attached, spread, and developed areas of both ES cell-like and endoderm-like cells, but that after prolonged culture only endoderm-like cells were evident. Id.

It has been determined earlier that ES cells, when injected into mouse blastocysts in vivo are incorporated into the ICM of the recipient embryo, and contribute to many different tissue types, including the germ line. Stewart et al., “Stem Cells from Primordial Germ Cells Can Reenter the Germ Line,” Dev. Biol. 161:626-8 (1984). See also, Bradley et al., Nature 309: 255-256 (1984).

Recently, Cherny et al., Theriogenology, 41:175 (1994) reported pluripotent bovine primordial germ cell derived cell lines maintained in long-term culture. After approximately seven days in culture, such cells produced ES-like colonies that stained positive for alkaline phosphatase (AP), exhibited the ability to form embryoid bodies, and spontaneously differentiated into at least two different cell types. These cells also reportedly expressed mRNA for the transcription factors OCT4, OCT6 and HES1.

Campbell et al., Theriogenology, 43:181 (1995) (abstract) reported the production of live lambs following nuclear transfer of cultured embryonic disc (ED) cells from day nine ovine embryos, which were cultured under conditions that promote the isolation of ES cell lines in the mouse. Based on their results, the authors concluded that ED cells from day nine ovine embryos are totipotent by nuclear transfer, and that totipotency is maintained in culture for up to three passages. Campbell et al., Nature, 380:64-68 (1996), further reported cloning of sheep by nucleic transfer from a cultured cell line.

Van Stekelenburg-Hamers et al., Mol. Reprod. Dev., 40:444-454 (1995), reported the isolation and characterization of purportedly permanent cell lines from ICM cells of bovine blastocysts. The authors isolated and cultured ICMs from 8- or 9-day bovine blastocysts under different conditions to determine which feeder cells and culture media are most efficient in supporting the attachment and outgrowth of bovine ICM cells. They concluded based on their results that the attachment and outgrowth of cultured ICM cells is enhanced by the use of STO (mouse fibroblast) feeder cells (instead of bovine uterus epithelial cells), and by the use of charcoal-stripped serum (rather than normal serum) to supplement the culture medium. Van Stekelenburg et al. report, however, that their cell lines resembled epithelial cells more than pluripotent ICM cells. Id.

Smith et al., WO 94/24274, published Oct. 27, 1994, Evans et al, WO 90/03432, published Apr. 5, 1990 and Wheeler et al, WO 94/26889 published Nov. 24, 1994, reported the isolation, selection and propagation of animal stem cells which purportedly may be used to obtain transgenic animals. Also, Evans et al., WO 90/03432, published on Apr. 5, 1990, reported the derivation of purportedly pluripotent embryonic stem cells derived from porcine and bovine species, for the production of transgenic animals. Further, Wheeler et al., WO 94/26884, published Nov. 24, 1994, disclosed embryonic stem cells, for the manufacture of chimeric and transgenic ungulates.

The use of ungulate ICM cells for nuclear transplantation has also been reported. Collas et al., Mol. Reprod. Dev., 38:264-267 (1994), for example, disclosed a technique of nuclear transplantation of bovine ICMs by microinjection of the lysed donor cells into enucleated mature oocytes. The reference disclosed culturing of embryos in vitro for seven days to produce fifteen blastocysts, which upon transferal into bovine recipients, resulted in four pregnancies and two births. Also, Keefer et al., Biol. Reprod., 50:935-939 (1994), disclosed the use of bovine ICM cells as donor nuclei in nuclear transfer procedures to produce blastocysts, which resulted in several live offspring upon transplantation into bovine recipients. Further, Sims et al., Proc. Natl. Acad. Sci., USA, 90:6143-6147 (1993), disclosed the production of calves by transfer of nuclei from short-term in vitro cultured bovine ICM cells into enucleated mature oocytes.

The production of live lambs following nuclear transfer of short-term cultured embryonic disc cells (up to three passages) has been reported (Campbell et al., Theriogenology, 43:181 (1995)). Further, the use of bovine pluripotent embryonic cells in nuclear transfer and the production of chimeric fetuses have also been reported (Stice et al., Theriogenology, 41:301 (1994)).

More recently, Cibelli et al, WO 01/29206, published Apr. 26, 2001, assigned to Advanced Cell Technology (ACT), disclosed methods for differentiating mammalian ES cells, including human, isolated from the inner cell mass of blastocysts to generate cells and organs for isogenic, allogenic, and/or xenogeneic transplantation. However, the stem cells disclosed were created from fertilized embryos unlike the present invention. Moreover, efforts to create stem cell from non-fertilized embryos by investigators at ACT were unsuccessful, see Washington Post, “First Human Embryos Are Cloned in US,” Nov. 26, 2001.

Based on the foregoing, it is evident that many groups have attempted to produce ES cell lines. The attention that ES cells have received is primarily because ES cells are pluripotent, and therefore can give rise to mature, differentiated, functional cells. Despite the promising therapeutic and prophylactic application of ES cells, however, use of ES cells raises various ethical concerns. ES cells, as described in the foregoing paragraphs, are derived from blastocysts that develop upon fertilization of an oocyte. Hence, ES cells are inherently derived, or harvested, from potentially viable embryos that are created expressly to be sacrificed.

Moreover, there are technical problems associated with use or development of ES cells. For example, ES cells derived from other individuals, e.g., from cell lines currently in existence, may cause immunoreactivity when transplanted into an incompatible recipient, and ES cell lines derived from somatic nuclear transfers may be less than ideal for therapeutic uses, since genetic mutations acquired during the lifetime of the nuclear donor will be carried into the pluripotent cell lines.

However, pluripotent cells, which include ES cells, are enormously useful because they can be used therapeutically to treat diseases like genetic diseases, neurodegenerative diseases, and cancer, for example, by repairing or restoring function to damaged nerves, or by providing a source of replacement tissues or organs. Pluripotent cells can also be used in the study of developmental biology, and for transplantation therapies because of their ability to give rise to germline chimeras or transfer their genome into the next generation.

The development of other sources of pluripotent cells is hence needed in the art. The present invention provides one such source. In one embodiment, the present invention provides isolated homozygous stem (HS) cells that are isolated from a blastocyst-like mass that is created by: (a) fusing two oocytes or two spermatids; (b) preventing the extrusion of the second polar body during oogenesis; (c) allowing the extrusion of the second polar body and spontaneous genomic self-replication in appropriate conditions; or, (d) transferring two haploid egg or sperm nuclei into an enucleated oocyte. Additionally, screening for stem cells that are homozygous is performed using genotyping when method (a) or (d) are used.

The HS cells of the present invention are pluripotent, and raise no ethical concerns as they isolated from cell-masses that are non-fertilized, and incapable of developing into viable embryos. Moreover, immunohistocompatibility matching is difficult to accomplish when heterozygous ES cell lines are employed in tissue or cell transplantation therapy, or maintained in banks and/or depositories. This is because the ES cell lines, including those developed by Advanced Cell Technology and other organizations, are derived from fertilized embryos or from nuclear transfer techniques using adult differentiated cells, and are genomically heterozygous. Because the pluripotent stem cells of the present invention are homozygous (with minimal heterozygosity or uniform homozygosity), such cells may be used to overcome immunohistocompatibility problems faced by currently available transplantation, cell replacement, and gene therapy techniques employing ES cell lines, or maintaining ES cell line banks and/or depositories.

During gametogenesis, heterozygous germ cells, i.e. germ cells with both paternal and maternal chromosomes, undergo meiosis. In the first meiotic division (meiosis I), homologous chromosomes separate to form two homozygous daughter cells that contain either paternal or maternal chromosomes with some heterozygosity introduced because of the phenomenon of crossing-over. Further, during oogenesis, the extrusion of one daughter cell (the primary polar body) is observed. The other daughter cell is arrested at metaphase II. Such metaphase II diploid oocytes may be used to derive homozygous stem cells with minimal heterozygosity.

Upon proper activation, a metaphase II oocyte can proceed to complete meiosis by the extrusion of one of chromatid (i.e. the secondary polar body) and give rise to a haploid cell. Such meiosis-completed haploid oocyte self-replicates without cytokinesis, rendering it diploid and uniformly homozygous. Such meiosis-completed haploid oocytes, hence, may also be used to create the homozygous stem cells of the present invention with no heterozygosity. See also, Kaufman M. H., Robertson E. J., Handyside A. H., Evans M. J., “Establishment of pluripotential cell lines from haploid mouse embryos,” J. Embryol. Exp. Morphol., 73:249-61 (1983).

Both HS cells with minimal heterozygosity and uniform homozygosity are superior to stem cells with heterozygous ES cells (such as those derived from using fertilized embryonic embryos, therapeutic cloning embryos, and adult stem cells) in that homozygous stem cells can contain two sets of identical Major Histocompatibility Complex (MHC) haplotypes. Therefore, immunohistocompatibility matching between a donor and an individual in need of transplantation therapy is easier to achieve with HS cells. Such stem cells homozygous for one MHC haplotype are tolerated not only by recipients carrying the identical haplotype, but also by recipients with the same MHC components in either of their parental haplotypes.

Furthermore, human MHC loci are within 4 Mb on chromosome 6, and MHC alleles are usually inherited en bloc. Some MHC allelic combinations are shared in a considerably higher frequency in the population, for example the 15 most common HLA-A, -B, -DR haplotypes are shared by 21.3% Caucasian Americans, and similar observations of haplotype frequency are seen in other ethnical backgrounds, Mori, M., et al., “HLA gene and haplotype frequencies in the North American population: the National Marrow Donor Program Donor Registry,” Transplantation, 64(7):1017-27 (1997). Considering such evidence supporting such linkage disequilibrium, the use of non-fertilized post-meiosis I diploid gamete derived HS cells can reduce the number of immunologically different cell lines needed to be maintained in a stem cell bank or depository for tissue or cell transplantation.

Hence, potentially, a few hundred stem cell lines that are homozygous for different haplotypes will be sufficient to match a majority of the population. This number is tremendously smaller in contrast to the number of haplotypes needed to maintain a bank or depository for stem cell lines derived from embryonic stem cells, adult stem cells, or therapeutic cloning stem cells. For example, for every 200 haplotypes there are more than 20,000 heterozygous possibilities.

The present invention, therefore, in one embodiment, provides stem cells homozygous for MHC loci and a wild-type (normal) gene that can be derived from nonfertilized oocytes from female donors related to a recipient to treat hereditary diseases, for example, hemophilia, diabetes, Huntington's, and so forth. The advantage of excluding an abnormal (disease-causing) allele in the HS cell lines of the present invention cannot be achieved at this time by currently available ES cell lines.

Teratomas are benign tumors that are composed of a variety of tissue elements reminiscent of normal derivatives from any of the three germ layers. Naturally found teratomas are derived from diploid totipotent cells, typically non-fertilized germ cells, having the capacity to differentiate into elements representative of any of the three germ layers—ectoderm, mesoderm, and endoderm. Scientific theories on the origin of teratomas include incomplete twinning, neoplastic proliferation of sequestered totipotent blastomeres or primordial germ cells, de-repression of totipotent generic information in the nuclei of somatic cells, and parthenogenetic development of germ cells.

Naturally occurring spontaneous teratomas are diploid and occasionally polyploid (Surti et al., Am. J. Hum. Gene. 47:635-643 (1990)). It is believed that diploid teratomous tissue occurs secondary to meiosis I, or due to fusion of the second polar body with the ovum (Eppig and Eicher, Genetics, 103:797-812 (1983); Eppig and Eicher, J. Hered., 79:425-429 (1988)). Further, teratomas have been proved to be genetically homozygous in heterozygous hosts (Linder, Proc. Natl. Acad. Sci. USA, 63:699-704 (1969); Linder and Power, Ann. Hum. Genet. 34:21-30, (1970); Linder et al., Nature, 254:597-598 (1975); Kaiser-McCaw et al., Cytogenet. Cell. Genet., 16:391-395 (1975)). Subsequent studies, however, failed to consistently replicate such results (Surti et al., Am. J. Hum. Gene., 47:635-643 (1990); Carritt et al., Proc. Natl. Acad. Sci. USA, 79:7400-7404 (1982); Parrington et al., J. Med. Genet., 21:1-12 (1984); Deka et al., Am. J. Hum. Genet., 47:644-655 (1990); Dahl et al, Cancer Genet. Cytogenet., 46:115-123 (1990)).

Compared to other tumors, teratomas exhibit unique histological features. They are composed of various differentiated tissues, including tissues such as epidermis, central nervous system tissue, or mature cartilage. They also contain nonspecific tissue types, e.g., lymphoid tissue or fibrous stroma. A “stemplasm” is a newly derived term used to describe a mass that develops upon the transplantation of HS cells into a host. Unlike teratomas, a stemplasm exhibits controlled growth, while still containing cells from all three embryonic germ layers. It can therefore be used as a means for the in vivo differentiation of the HS cells of the present invention.

There is clearly a need in the art for a reliable source of stem cells capable of directed differentiation. The present invention fulfills this need by providing homozygous stem cells without the necessity of fertilization procedures. The present invention discloses homozygous stem (HS) cells derived from non-fertilized post-meiosis I diploid germ cells. Donor cells, which may be harvested from an individual donor using techniques commonly used in the field of in vitro fertilization, can be induced to form blastocyst-like masses from which the HS cells of the present invention can be derived, and such HS cells can be differentiated into any cell type, group of cells, or tissue type. Further, HS-derived differentiated cells and/or tissues may be used subsequently for diagnosis and treatment, particularly cell replacement therapy and gene therapy, and cosmetic and/or therapeutic transplantation. Such uses, moreover, are intended to be exemplary rather than exhaustive.

### III. SUMMARY OF THE INVENTION

The present invention relates to the production of isolated homozygous stem cells (HS), and the discovery that these cells have the unique property of being able to be differentiated in a directed and predictable manner. In this way, HS cells mimic ES cells, but do not require fertilization procedures, or harvesting of embryonic tissue.

It is an object of the invention to provide novel and improved methods for producing isolated homozygous stem cells, which can be used as sources of cells for cell therapy and for the generation of cells, masses of cells, tissues and organs for transplantation.

It is an object of the invention to provide isolated homozygous stem (HS) cells. It is a further object of the invention to provide HS cells derived from animal donor material, including animals of the following species: mammals, birds, fish, amphibians, and reptiles. In one preferred embodiment, the animal is a mammal, more preferably a human. HS cells are derived from non-fertilized post-meiotic I diploid germ cells retrieved from donors, where donor cells may be harvested using current and future in vitro fertilization techniques.

It is another object of the invention to provide homozygous stem cells (HS) derived from blastocyst-like masses mitotically created by: (a) fusing two oocytes or two spermatids; (b) preventing the extrusion of the second polar body during oogenesis; (c) allowing the extrusion of the second polar body and spontaneous genomic self-replication in appropriate conditions; or, (d) transferring two haploid egg or sperm nuclei into an enucleated oocyte. Additionally, screening for stem cells that are homozygous is performed using genotyping when method (a) or (d) are used.

It is also an object of the invention to provide methods of deriving homozygous stem cells from non-fertilized post-meiosis I diploid germ cells. Preferably, HS cells are derived using methods for preventing the extrusion of the second polar body from an oocyte during oogenesis, or allowing the extrusion of the second polar body and spontaneous genomic self-replication under appropriate conditions of such haploid oocyte to create a blastocyst-like mass from which HS cells are extracted.

In one embodiment, homozygous stem cells are derived from metaphase II oocytes. Preferably, oocytes are allowed to mature to metaphase II in vivo, and recovered using methods commonly used in in vitro fertilization. Such metaphase II oocytes are then mitotically activated after allowing a resting period of between extracted.about 0-9 hours. In humans, the resting period is between 5-9, preferably about 3-5, more preferably about 1 1/2-3, and most preferably between 0-1 /2 hours after egg retrieval from a donor. Moreover, frozen human oocytes that are matured to metaphase II in vivo or in vitro may also be used to derive the HS cells of the present invention. In a further embodiment, oocytes, excluding bovine or primate oocytes, may be matured to metaphase II in vitro, where maturation times longer or shorter than 36 hrs may be required for some animals.

HS cells created upon activation of non-fertilized post-meiosis I diploid germ cells form stemplasms when transplanted into a live animal. It is a further object to isolate HS cells, and multi-potent progenitor cells from said stemplasms. It is another object of the invention to provide methods of selecting the cell to be isolated from said stemplasm.

It is another object of the invention to provide a method of making a desired cell, group of cells, or tissue type comprising directing the differentiation of an isolated HS cell as described above, under suitable conditions, so as to arrive at the desired cell, group of cells, or tissue type. It is a further object of the invention to provide differentiated cells derived from HS cells, and use such differentiated cells for therapy and/or diagnosis. Exemplary tissues include, but are not limited to, tissues of the epithelium, connective tissue, muscle tissue or nervous tissue.

Illustrative types of epithelial cells include but are not limited to keratinizing epithelial cells; wet-stratified barrier epithelia; lining epithelial cells; exocrine-secreting epithelial cells; endocrine-secreting epithelial cells; extracellular matrix-secreting epithelial cells; absorptive epithelial cells, such as those of the gut, exocrine glands, and urogenital tract; and contractile epithelial cells. Illustrative types of connective tissue cells include but are not limited to extracellular matrix-secreting cells; cells specialized for metabolism and storage; and circulating cells of the blood and immune systems. Illustrative types of muscle cells include but are not limited to contractile cells and ciliated cells with propulsive function. Illustrative types of nervous or sensory cells include but are not limited to: a) sensory transducers; b) autonomic neurons; c) supporting cells of sense organs; and d) peripheral neurons; and neurons and glial cells of central nervous system. Illustrative types of reproductive cells include but are not limited to germ cells and nurse cells.

It is a more specific object of the invention to provide novel methods for inducing cells derived from HS cells to differentiate into multi-potent progenitor cells which can be also be used as sources of cells for diagnosis, treatment, for example, cell therapy, gene therapy, and for the generation of cells, masses of cells, tissues and organs for transplantation. Such uses, moreover, are exemplary rather than exhaustive.

It is an object of the invention to provide improved methods for producing genetically engineered progenitor cells derived from HS cells, which can be used as a source for diagnosis, treatment, for example, cell therapy, gene therapy, and for the generation of cells, masses of cells, tissues and organs for transplantation. Such uses, moreover, are exemplary rather than exhaustive. In one embodiment, a desired gene may be inserted, removed or modified in HS cells that are caused to further differentiate into progenitor cells. In a further embodiment, the progenitor cell itself may be genetically altered and then cultured to generate colonies of genetically altered progenitors.

It is another object of the invention to provide progenitor cells, preferably human progenitor cells derived from HS cells. Such progenitors, in one embodiment, are induced to differentiate into cells, groups of cells, tissues and/or organs. Further, it is an object of the invention to use such progenitor cells to culture differentiated cells and/or tissues for therapy and/or diagnosis.

It is a specific object of the invention to provide progenitor cells, preferably human, for treatment or diagnosis of any disease wherein cell, tissue or organ transplantation, gene therapy and/or cell therapy is therapeutically or diagnostically beneficial. The HS cells, progenitor cells, and or differentiated cells of the present invention may be used within the same species or across species.

The HS and progenitor cells, and further differentiated HS and progenitor cells of the present invention may be created using ova or sperm of the same, related or unrelated mammals, preferably human.

It is another specific object of the invention to use the differentiated cells produced according to the invention in vitro or in vivo for the study of cell differentiation and for assay purposes, for example for drug studies.

It is another object of the invention to provide models of disease states for use in investigating or diagnosing same using genetically modified HS cells, or groups of cells, tissues or organs generated from isolated HS cells.

It is another object of the invention to provide a method of treating a disorder or disease state by generating, in situ or in vitro, suitable replacement cells, groups of cells, tissues or organs from isolated HS cells. Illustrative disorders and disease states include but are not limited to traumatic injury (e.g., post-trauma repair and reconstruction, for limb replacement, spinal cord injury, burns, and the like) and birth defects; pathological and malignant conditions of the cells, tissues, and organs (e.g., cancer); and degenerative and congenital diseases of the cells and tissues of the muscles (e.g., muscular dystrophy, cardiac conditions), nerves (e.g., Alzheimer's, Parkinson's, and multiple sclerosis), epithelium (e.g., blindness and myopathy, atherosclerosis and other stenotic vascular conditions, enzyme deficiencies such as Crohn's disease, and hormone deficiencies such as diabetes), and connective tissues (e.g., immune conditions and anemia). HS-derived cells and tissues may be grafted or transplanted to a subject in need, preferably using the subject's own donor material.

It is another object of the invention to provide improved methods of diagnosis and transplantation, gene and/or cell replacement therapy comprising the usage of isogenic or syngenic cells, tissues, or organs produced from differentiated cells produced according to the invention. Such therapies by way of example include treatment of diseases and injuries including Parkinson's, Huntington's, Alzheimer's, ALS, spinal cord injuries, Multiple Sclerosis, Muscular Dystrophy, diabetes, liver diseases, heart disease, cartilage replacement, burns, vascular diseases, urinary tract diseases, as well as the treatment of immune defects and cancer, and bone marrow transplantation.

These and further objects of the invention are fully described by the below detailed description, examples, and claims.

### IV. BRIEF DESCRIPTION OF THE FIGURES

The patent or application file contains at least one drawing executed in color. Copies of this patent or patent application publication with color drawings will be provided by the Office upon request and payment of the necessary fee. See 37 C.F.R. §1.84(a)(2)(iv).

FIG. 1: Products of parthenogenetic activation of oocytes.

FIG. 2: A schematic representation of spermatogenesis and oogenesis.

FIG. 3: Fusion of oocytes and development of oocyte fusion products.

FIG. 4: Detail of products of parthenogenetic activation of oocytes.

FIG. 5A: Photograph of the morphology of a colony forming unit (CFU) derived from mouse HS cells.

FIG. 5B: Photograph of the morphology of erythrocytes derived from mouse HS cells.

FIG. 5C: Photograph of the morphology of a monocyte derived from mouse HS cells.

FIG. 5D: Photograph of the morphology of lymphocyte derived from mouse HS cells.

FIG. 5E: Photograph of the morphology of hematopoietic cells with granules, derived from mouse HS cells.

FIG. 5F: Photograph of the morphology of hematopoietic cells with both granules and monocytes derived from mouse HS cells.

FIG. 6: Photograph of the morphology of beating muscle cells derived from mouse HS cells.

FIG. 7A: Photograph of the morphology of clusters of pancreatic cells derived from mouse HS cells.

FIG. 7B: Photograph of insulin and glucagon staining of pancreatic cells derived from mouse HS cells where insulin staining is shown in brown, and glucagon staining in red.

FIG. 8A: Photograph depicting the development of a morula-like mass derived from human homozygous post-meiosis I diploid oocytes.

FIG. 8B: Photograph of an early blastocyst-like mass derived from human homozygous post-meiosis I diploid oocytes.

FIG. 8C: Photograph of a blastocyst-like mass revealing the inner cell mass derived from human homozygous post-meiosis I diploid oocytes.

FIG. 8D: Photograph of an isolated inner cell mass growing on feeder layers (D) derived from human homozygous post-meiosis I diploid oocytes.

FIG. 9A: Photograph of the morphology of Nestin-positive neuronal precursor cells derived from mouse HS cells.

FIG. 9B: Photograph of the morphology of Tyrosine Hydroxylase-positive neuronal cells derived from mouse HS cells.

### V. DETAILED DESCRIPTION OF THE PREFERRED EMBODIMENTS

All references cited herein are hereby incorporated by reference in their entirety. Nothing herein is to be construed as an admission that the invention is not entitled to antedate such disclosure by virtue of prior invention or that the prior art provides an enabling or adequate disclosure. Throughout this description, the preferred embodiments and examples shown should be considered as exemplary, rather than as limitations on the present invention.

The present invention provides isolated homozygous stem (HS) cells, methods of producing HS cells, and methods for making differentiated cells for use in diagnosis, cell therapy, gene therapy, or as a source of cells to provide tissues and organs for cosmetic and therapeutic transplantation. Such uses, moreover, are not exhaustive. More particularly, HS cells are isolated from a blastocyst-like mass derived from non-fertilized post-meiosis I diploid germ cells.

In the past, embryonic stem (ES) cells were generated by long-term culture of cells derived from the inner cell mass of fertilized blastocysts. Subsequently, ES cells were cultured and genetically modified, and induced to differentiate in order to produce cells to make transgenic animals or cells for therapy.

The present invention differs from prior methods of obtaining pluripotent cells capable of differentiating, in that it provides stem cells that are homozygous, and isolated from blastocyst-like masses that are created upon the mitotic activation of non-fertilized post-meiosis I diploid germ cells. Moreover, HS cells isolated from the blastocyst-like mass may be induced to differentiate to obtain differentiated cells or tissue, multi-potent progenitor cells, or be maintained as permanent cell lines. If so desired, genetic modifications may be introduced into the HS cells or progenitor cells of the present invention.

Thus, the present invention provides pluripotent HS cells, multi-potent progenitor cells, and/or terminally differentiated cells, methods of making same, where such cells may be used for various therapeutic and diagnostic purposes.

A. Definitions

In the context of the present invention, the following definitions apply.

“Differentiation” is a highly regulated process that cells undergo as they mature into normal functional cells. Differentiated cells have distinctive characteristics, perform specific functions and are less likely to divide. Conversely, undifferentiated cells are rapidly dividing immature, embryonic or primitive cells having a nonspecific appearance with multiple nonspecific activities and functions.

As used herein, the term “stem cell” refers to a relatively undifferentiated cell that actively divides and cycles, giving rise upon proper stimulation to a lineage of mature, differentiated, functional cells. The defining properties of a stem cell include: (a) it is not itself terminally differentiated; (b) it can divide without limit for the lifetime of the animal; and (c) when it divides, each daughter has a choice of remaining a stem cell or embarking on a course that leads irreversibly to terminal differentiation. Those stem cells that are initially unrestricted in their capabilities (i.e., capable of giving rise to several types of differentiated cell) are called “pluripotent”. Current sources of pluripotent cells include embryonic (ES) stem cells, embryonic carcinoma (EC) cells, cells generated from somatic cloning, teratomas and teratocarcinomas.

Progenitor cell lines, each capable of producing cells from one of the three germ layers, i.e. the endoderm, mesoderm and ectoderm, are referred to in the present application as “multi-potent”. While each progenitor cell line is not terminally differentiated and can continue to divide for the lifetime of an animal, it is considered to be committed to different tissues or cells from only one type of embryonic layer. Therefore, particular progenitor cell lines may be differentiated into bone, cartilage, smooth muscle, striated muscle and hematopoietic cells (mesoderm); liver, primitive gut, and respiratory epithelium (endoderm); or, neurons, glial cells, hair follicles and tooth buds (ectoderm). The term “progenitor cells” hence may be used synonymously with “multi-potent stem cells” or “precursor cells”. Such progenitor cells lines, which are created by the directed differentiation of HS cells in vivo (where the term “in vivo” includes differentiation induced by encapsulating said HS cells in an isogenic or allogeneic animal to generate stemplasms from such encapsulated cells) or in vitro, can be maintained in culture as permanent cell lines.

A “teratoma” is a naturally occurring spontaneous mass of abnormal cells containing many types of differentiated tissue, tissues derived from all three embryonic layers, such as bone, muscle, cartilage, nerve, tooth-buds, glandular epithelium, and so forth, mixed with undifferentiated stem cells that continually divide and generate yet more of these differentiated tissues.

Additionally, a teratoma is a spontaneously formed neoplasm usually found in reproductive tissues, which contains cells from all the three embryonic germ layers. Further, it is characterized by unregulated growth. A “stemplasm” is a newly derived term used to describe a mass that develops upon the transplantation of HS cells into a host. Unlike teratomas, a stemplasm exhibits controlled growth, while still containing cells from all three embryonic germ layers. It can therefore be used as a means for the in vivo differentiation of the HS cells of the present invention.

A “teratocarcinoma” is secondary to a teratoma. Teratomas are largely benign; however if they become malignant, a teratocarcinoma develops and can be deadly to the host.

A “homozygous stem cell”, previously termed a “teratoma stem cell” or a “TS cell”, is an undifferentiated stem cell arising from a non-fertilized post-meiosis I diploid germ cell. Preferably, it is formed by preventing the extrusion of the second polar body during oogenesis (or “activation”), or allowing the extrusion of the second polar body and spontaneous genomic self-replication of the haploid oocyte in appropriate conditions. Homozygous stem (HS) cells are isolated cells generated from the inner cell mass of blastocyst-like masses that develop upon “mitotic activation” of non-fertilized post-meiosis I diploid germ cells, which can be accomplished by: (a) fusing two oocytes or two spermatids; (b) preventing the extrusion of the second polar body during oogenesis; (c) allowing the extrusion of the second polar body and spontaneous genomic self-replication in appropriate conditions; or, (d) transferring two haploid egg or sperm nuclei into an enucleated oocyte. Additionally, screening for stem cells that are homozygous is performed using genotyping when method (a) or (d) are used.

In mammalian development, cleavage produces a thin-walled hollow sphere, the “blastocyst”, with the embryo proper being represented by a mass of cells at one side, otherwise known as the “inner cell mass”. The blastocyst is formed before implantation and is equivalent to the “blastula”. The wall of the thin-walled hollow sphere is referred to as the “trophoblast”, which is the extra-embryonic layer of epithelium that forms around the mammalian blastocyst, and attaches the embryo to the uterus wall. The trophoblast forms the outer layer of the chorion, and together with maternal tissue will form the placenta.

In the context of the present invention, a “blastocyst-like mass” is different from a “blastocyst” (as used in the art) in that it is the product of a mitotically activated nonfertilized post-meiosis I germ cell.

As used herein, the term “mitotically activated” means acquiring the ability to undergo regular cell divisions mitotically, and includes both parthenogenetic activation of oocytes and androgenetic activation of spermatids. For the purposes of this application, mitotically activated is used synonymously with parthenogenic activation or androgenetic activation.

The term “homozygous post-meiosis I diploid germ cells”, as used herein, means germ cells that are the stage of gametogenesis at which the cells contain two copies of either the paternal or maternal homologous chromosomes.

B. Isolated Stem Cells of the Present Invention

As stated in the foregoing paragraphs, homozygous stem (HS) cells of the present invention arise from activated non-fertilized post-meiosis I diploid germ cells. For example, the fusion of two mature oocytes or spermatocytes results in a blastocyst-like mass from which HS cells may be derived. A stem cell derived from such blastocyst-like mass has a postmeiotic genotype rendering it homozygous, pluripotent, and biologically benign.

Furthermore, in a preferred embodiment, HS cells of the present invention can be procured from any individual and used in the same individual or a related or unrelated immunohistocompatible individual with high immunologic compatibility between the recipient and the HS cells, progenitors, or differentiated cells and/or tissues derived from the HS cells or progenitor cells.

HS cells can be induced to differentiate in vitro, or in vivo, into various types of tissues originating from all three germ layers. In a preferred embodiment, HS cells can be encapsulated in an allogeneic or isogenic animal to generate stemplasms, within which such cells can differentiate into various types of tissues originating from the endoderm, mesoderm, and ectoderm including, but not limited to, skin, hair, nervous tissue, pancreatic islet cells, bone, bone marrow, pituitary gland, liver, bladder, and other tissues having diagnostic or therapeutic utility in animals, including humans. Moreover, one skilled in the art of differentiation techniques, particularly those developed for differentiation of ES cells and embryonic carcinoma (teratocarcinoma) cells, can induce a pluripotent cell to differentiate into a desired type of tissue without undue experimentation.

For example, Hole, Cells Tissues Organs, 165: 181-189 (1999), incorporated by reference herein) describes methods for directing the differentiation of hematopoietic cells from embryonic stem cells in vitro. In addition, Doetschman et al., Embryol. Exp. Morphol., 87: 27-45 (1985), incorporated by reference herein) suggest that the withdrawal of leukemia inhibitory factor (LIF) from ES cells grown in suspended culture results in the formation of cystic embryoid bodies containing blood islands made up of erythrocytes and macrophages. The production of other hematopoietic cells, including neutrophils, mast cells, macrophages and erythroid cells, from stem cells has also been described. (See, e.g., Wiles and Keller, Development, 111: 259-267 (1991); Keller et al, Mol. Cell. Biol. 13: 473-486 (1993a); and Lieschke and Dunn, Exp. Hematol., 23: 328-334 (1995), each of which are hereby incorporated by reference herein in their entirety). Such methods are applicable to HS cells of the present invention The techniques described by Cho et al., Proc. Natl. Acad. Sci. USA, 96:9797-9802 (1999), incorporated by reference herein, for efficiently differentiating ES cells into mature Ig-secreting B lymphocytes can also be adapted for use with the HS cells of the present invention. Likewise, Dani, Cells Tissues Organs, 165: 173-180 (1999), also incorporated by reference herein, describes a method for cells, differentiating ES cells into adipocytes serves as a promising model for use in the context of the present invention. For example, the treatment of embryoid bodies at an early stage of their differentiation with retinoic acid (RA) for a short period of time appears to be linked to adipogenesis.

Techniques for eliciting the differentiation of stem cells into a variety of neuronal cells are described by Okabe et al. Mech. Dev., 59: 89-102 (1996), incorporated by reference herein). Likewise, McDonald et al., Nature Medicine, 5:1410-1412 (1999), incorporated by reference herein, describe oligodendrocytes and neurons derived from stem cells that have particular use in treating injured spinal cords. These techniques can be used with HS cells of the present invention.

The use of accessory cell lines, such as OP9, to derive particular cell lineages is also contemplated. See, for example, Nakano et al., Science, 265:1098-1101 (1994), incorporated by reference herein, and Nakayama et al., Blood, 91: 2283-2295 (1998), incorporated by reference herein, relating to erythroid, myeloid and lymphoid lineages.

Techniques for eliciting the differentiation of HS cells of the present invention into follicular cells, as well as epidermal cells are also contemplated. For example, Taylor et al., Cell, 102: 451-361 (2000), incorporated by reference herein, describe ES cell-derived follicle and epidermis cells may be used for hair replacement and skin graft therapies. These techniques can be adapted for use with the HS cells of the present invention. The expression of particular regulatory genes may also be used to direct differentiation. See, for example, Hole et al., Blood, 90:1266-1276 (1996a), and Battieres. Clin. Hematol., 3:467-483 (1997), incorporated by reference herein, relating to hematopoletic genes. Likewise, preliminary evidence suggests that nuclear regulatory factors involved in lipid metabolism, including but not limited to PPARs (PPARδ and PPARγ) and C/EBPδ (C/EBPβ, C/EBPδ and C/EBPα), may also be triggers of terminal differentiation of preadipocytes into adipocytes. Such factors would find utility in the context of the differentiation methods of the present invention.

Depending on the function needed, differentiation may be assessed by detecting expression of a gene specific for differentiation, by detecting tissue-specific antigens, by examining cell or tissue morphology, by detecting functional expression such as ion channel function; or by any means suitable for detecting the differentiation of HS cells.

Multi-potent progenitor cells, derived from the HS cells of the present invention by in vivo or in vitro directed differentiation techniques, are capable of producing cells from all three germ layers: the endoderm, mesoderm and ectoderm. For example, progenitor cells may be differentiated into bone, cartilage, smooth muscle, striated muscle and hematopoietic cells (mesoderm); liver, primitive gut, and respiratory epithelium (endoderm); or, neurons, glial cells, hair follicles and tooth buds (ectoderm). While it is not necessary for progenitor cells of the present invention to be immortal, they may be maintained as immortal lines. Morphologically, progenitor cells do not express cell surface markers found on ES cells, such as cell surface markers characteristic of primate ES cell lines-positive for SSEA-3, SSEA-4, TRA-1-60, TRA-1-81, alkaline phosphatase activity, and negative for SSEA-1.

Preferably, culturing HS cells in the absence of other pluripotent HS cells leads to the production of progenitor cells of the present invention. Moreover, the proliferation of progenitor cells is aided by preventing further growth and proliferation of pluripotent HS cells from which said progenitor cells are derived. Techniques known in the art can be used to generate progenitor cells, for example, HS cells isolated from a blastocyst-like mass can be cultured in the presence of differentiation-inducing agents, and in the absence of other HS cells to produce multi-potent progenitor cells and prevent undifferentiated HS cells from proliferating further.

Further, the isolated HS cells of the present invention are incapable of developing into full-term embryos because of genomic imprinting; however, HS retain their ability to differentiate into functional differentiated cells, and/or tissues as demonstrated in the examples that follow. The mechanism of genomic imprinting is at present poorly understood, however it has been clearly demonstrated that parthenogenetic embryos fail to develop to term as a consequence of imprinting (Surani et al, Development Supplement, 89-98 (1990)). Imprinting involves a germline-specific epigenetic marking process since the expression of imprinted genes is determined by their parental origin (Allen et al., Development, 120: 1473-1482 (1994)). Heritable epigenetic modifications that could be employed in imprinting mechanisms include allele-specific DNA methylation and chromatin structural modifications such as those detected by DNase I hypersensitivity assays. Id. For certain genes (e.g., Igf2r and H19), the paternal allele is imprinted, while for other genes (e.g., Igf2 and Snrpn) the mother's allele is always imprinted. Id. (See also, Mann et al., Cell, 62:251-260 (1990), and Devel. Biol., 3:77-85 (1992), incorporated by reference herein for a discussion of the pluripotency of androgenetic and parthenogenetic embryos, and the implications for genetic imprinting.)

C. Creation of Homozygous Stem Cells of the Present Invention

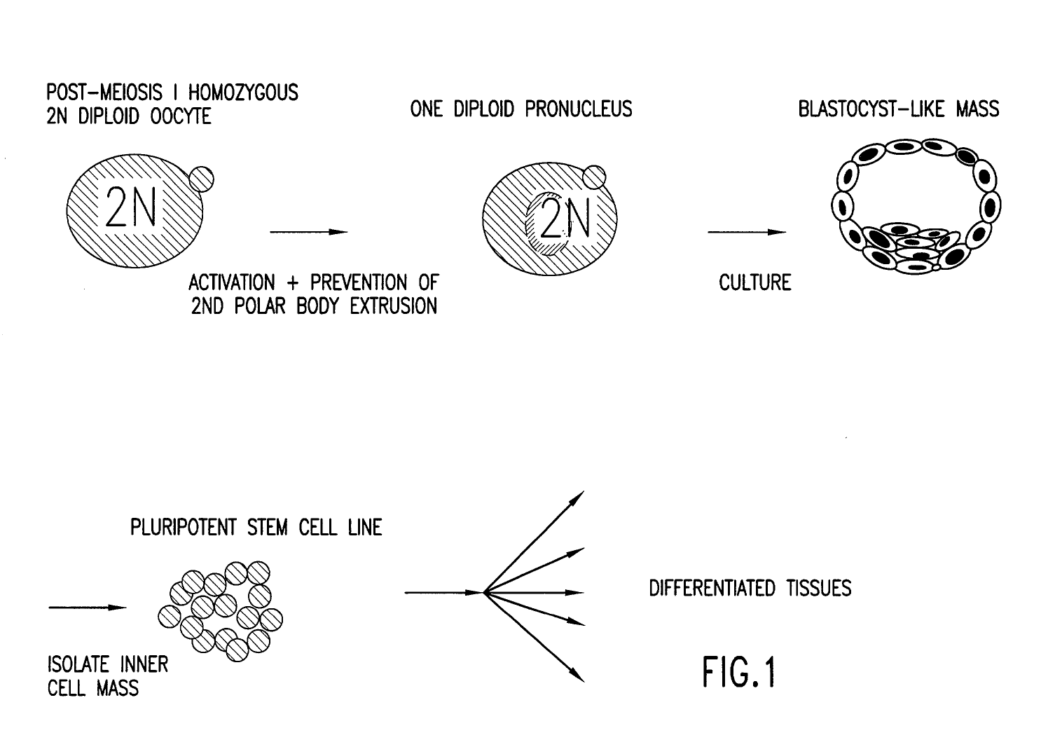

As noted above, the HS cell is isolated from a blastocyst-like mass that develops upon the mitotic activation, or creation of a non-fertilized post-meiosis I diploid germ cell. FIG. 1 provides a flow chart, showing a preferred method of developing HS cells from a non-fertilized post-meiosis I diploid germ cell.

Germ cells develop into non-fertilized post-meiosis I diploid germ cells that upon activation produce blastocyst-like masses from which the HS cells of the present invention are derived. HS cells, and/or differentiated cells, of the present invention find utility in the diagnosis and/or treatment of diseases, for example, by implantation or transplantation to an affected individual in need of such therapy.

While homozygous post-meiosis I diploid germ cells may be obtained from the same individual or from an immunocompatible donor, in certain situations self-donors are preferred. However, in cases where the affected individual selected for therapy suffers from a genetic disease (i.e., a disease characterized by a lack of a crucial gene, either due to mutation or improper expression), it may be preferable to utilize a non-self donor. Alternatively, one skilled in the art of selections procedures may choose those self germ cells that display the desired genotype (e.g., cells lacking a flawed or mutated gene), those cells capable of expressing the deficient gene. Such selection techniques may also be used to avoid an immuno-incompatible genotype or phenotype for tissue transplant.

Homozygous post-meiosis I diploid germ cells can be harvested from a donor using conventional technology, particularly those techniques commonly used in the field of in vitro fertilization. See, for example, Jones H W Jr. et al., Fertil. Steril., 37(1):26-29 (1982), describing techniques for aspirating oocytes from human ovarian follicles; Lisek et al., Tech. Urol., 3(2):81-85 (1997), describing techniques for collecting sperm from the epididymis and testicle; and Stice et al., Mol. Reprod. Dev., 38(1):61-8 (1994), and Takeuchi et al., Hum. Reprod., 14(5):1312-7 (1999), describing techniques for transplanting nuclear material of one donor to an enucleated oocyte of another. The entire contents of these references are hereby incorporated by reference herein.

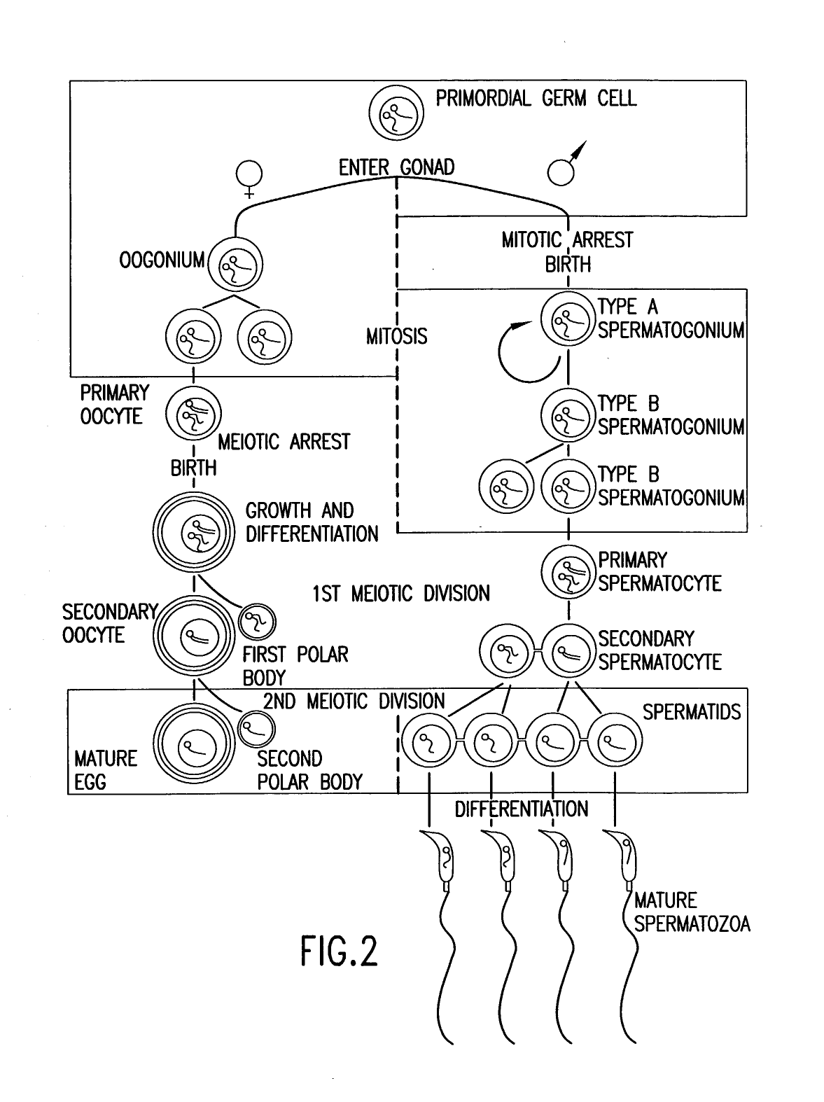

HS cells are by: (a) fusing two oocytes or two spermatids followed by screening for homozygous stem cells by genotyping; (b) preventing the extrusion of the second polar body during oogenesis; (c) allowing the extrusion of the second polar body and spontaneous genomic self-replication in appropriate conditions; or, (d) transferring two haploid egg or sperm nuclei into an enucleated oocyte followed by screening for homozygous stem cells by genotyping. FIG. 2 provides a schematic representation of spermatogenesis and oogenesis, showing the difference in phases of mitosis and meiosis in males and females.

Oocytes useful in the context of the present invention may be obtained using any suitable method known in the art, or yet to be discovered. Human oocytes are typically harvested from the ovarian follicles of a donor individual and isolated from surrounding or adhering cells. To maximize yield, superovulation is induced in the donor individual. Superovulation may be induced by the administration of appropriate gonadotropins or gonadotropin analogues, administered either alone or in combination with clomiphene citrate (Barriere et al., Rev. Prat., 40(29):2689-93 (1990), incorporated by reference herein). In mice, an exemplary method involves the administration of pregnant mare's serum (PMS) to mimic follicle-stimulating hormone (FSH) and human chorionic gonadotropin (hCG) to mimic luteinizing hormone (LH) (See Hogan et al., Manipulating the mouse embryo: A Laboratory Manual, 2^{nd} ed. Cold Spring Harbor Laboratory Press, 1994). Efficient induction of superovulation depends on several variables including, but not limited to, the age and weight of the female, the dose of gonadotropin, the time of administration, and the strain used.

Superinduction of ovulation and harvesting of oocytes are known in the art. For exemplary detailed mouse protocols, see Hogan et al., supra, pp. 130-132, the entire contents of which are hereby incorporated by reference. For example, Hogan describes the intraperitoneal administration of PMS and hCG, both resuspended from lyophilized powder in sterile 0.9% NaCl, to induce superovulation. Both PMS and hCG should be administered prior to the release of endogenous LH. The Hogan protocols, directed to the harvesting of oocytes from mice, can be routinely adapted for humans without undue experimentation.

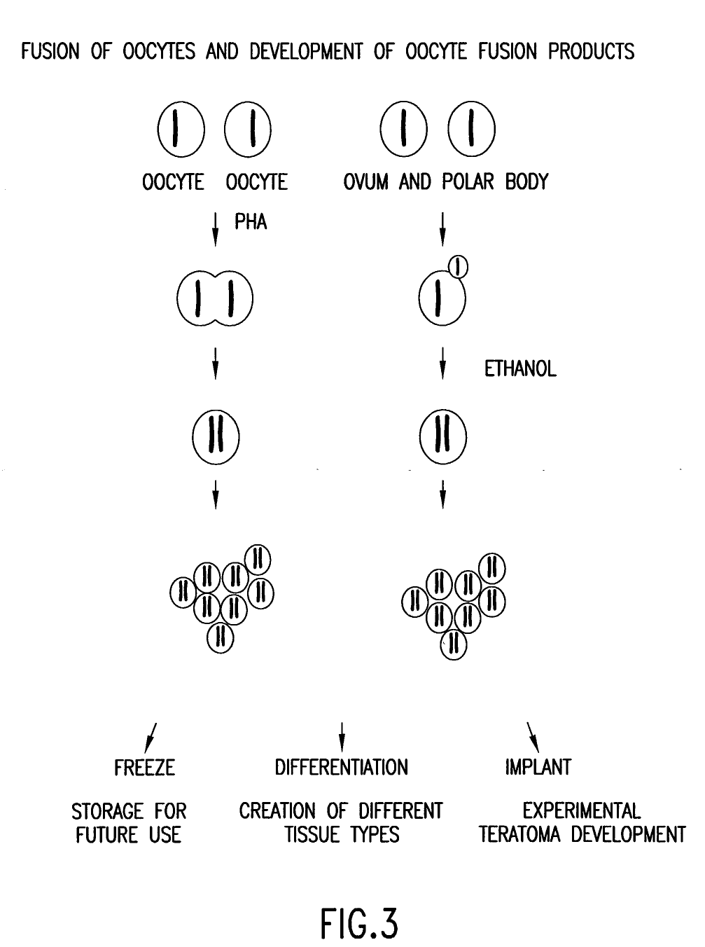

Polyethylene glycol has also been shown to induce fusion of ovulated oocytes (see, e.g., G G Sekirina, Ontogenez, 16(6):583-8 (1985), and Gulyas B J, Dev. Biol. 101(1):246-50 (1984), incorporated by reference herein). Alternatively, Nogues et al., Zygote, 2(1):15-28 (1994), incorporated by reference herein) describes the induction of oocyte fusion by inactivated Sendai virus, resulting in the production of “zygotes” or “oocyte fusion products (OFP)” that are able to undergo the first stages of embryonic development. For a review of oocyte fusion techniques, see Gulyas B J, Dev. Biol., 4:57-80 (1986), incorporated by reference herein. For a detailed protocol for fusion of mouse oocytes, see Hogan et al. supra, pp. 148-150, wherein harvested eggs with their cumulus cells attached are maintained in a solution of 7% ethanol in Dulbecco's PBS for 5 minutes, washed with medium, and incubated at 37° C. for 5 hours. The cumulus cells are subsequently removed by treatment with hyaluronidase. FIG. 3 provides a depiction of the fusion of oocytes and the development of oocyte fusion products.

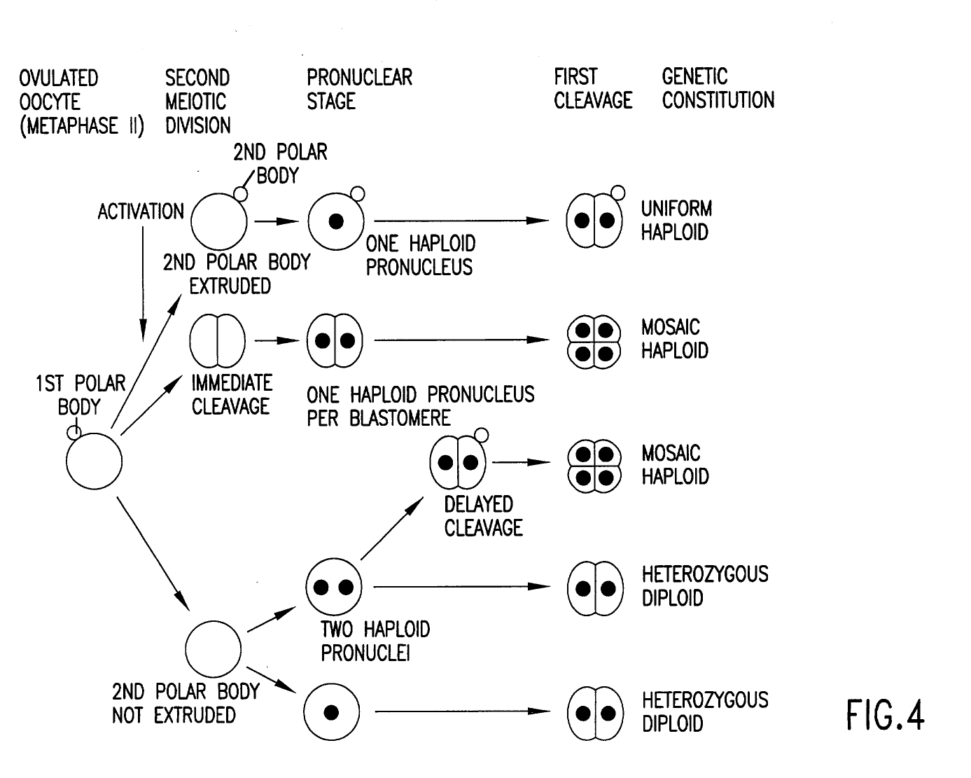

Alternatively, preventing the extrusion of the second polar body from oocytes can generate HS cells. In nature, following the first meiotic division and separation of the first polar body, the second meiotic division occurs. Exposing oocytes before the extrusion of the second polar body to agents including, but not limited to, Ca⁺⁺ ionophore (A23187), or ethanol, followed by exposure to agents including 6-dimethylaminopurine (6-DMAP), puromycin, or cytochalasin D, results in the activation of such diploid oocytes and subsequent formation of blastocyst-like masses. FIG. 4 depicts the possible products of activation.

In another embodiment, allowing the extrusion of the second polar body and spontaneous genomic self-replication may be used to derive HS cells. Upon parthenogenetic activation, oocytes extrude the secondary polar body and become haploid. Such haploid oocytes when incubated under appropriate conditions divide and form blastocyst like masses. See, Taylor, A. S., et al., “The early development and DNA content of activated human oocytes and parthenogenetic human embryos,” Hum. Reprod., 9(12):2389-97 (1994); Kaufman, M. H. et al., “Establishment of pluripotential cell lines from haploid mouse embryos,” J. Embryol. Exp. Morphol, 73:249-61 (1983).

Spermatids useful in the context of the present invention can be obtained using any suitable method known in the art or yet to be discovered, particularly those conventional in the field of in vitro fertilization. To create HS cells for use in a male, spermatids (meiosis II completed) are harvested and then induced to fuse. Spermatid fusion can be achieved using well-established standard techniques. For example, Asakura S, et al., Exp. Cell. Res., 181(2):566-73 (1989), incorporated by reference herein, teaches the use of a hypotonic medium to induce the fusion of a pair of spermatids and the eventual formation of a single acrosome (synacrosome). Alternately, secondary spermatocytes (meiosis I completed) can be activated using methods that are known in the art.

Finally, as noted above, the isolated HS cell can be created from an enucleated oocyte. Specifically, two sperm or haploid egg nuclei can be transferred into an enucleated oocyte to create a non-fertilized diploid oocyte bearing the nuclear genetic information of the donor male or female in the oocyte cytoplasm. In males, this approach favors paternal gene expression because it mimics the processes involved when a sperm fertilizes an ovum, which triggers gene expression in the zygote. The donor nuclear material can be harvested and/or isolated using standard techniques conventional in the art. Likewise, the transfer step can be performed using techniques conventional in the art of in vitro fertilization (see U.S. Pat. No. 5,945,577, WO98/07841 and the teachings of S. L. Stice and T. Takeuchi discussed above, and Wobus et al., Cells Tissues Organs, 166:1-5 (2000) that are incorporated by reference herein).

Genetic modifications may be introduced into HS cells by polynucleotide transfection techniques, including but not limited to, viral vector transfer, bacterial vector transfer, and synthetic vector transfer (e.g., via plasmids, liposomes and colloid complexes).

Methods for isolating ES cells from the inner cell mass of fertilized blastocysts are known in the art. Such methods may be adapted for isolating HS cells from the inner cell mass of blastocyst-like masses. For example, see Gardner et al., “Culture and Transfer of Human Blastocysts”, Current Opinions in Obstetrics and Gynecology, 11:307-311 (1999), U.S. Pat. No. 5,843,780 (Thomson et al.) and U.S. Pat. No. 5,905,042 (Stice et al.) the contents of which are incorporated by reference herein.

D. Deriving Progenitor Cells and Differentiated Cells, Masses of Cells and Tissue Types from the Isolated HS Cells of the Present Invention

Isolated HS cells are induced to differentiate in the absence or presence of cytokines, growth factors, extracellular matrix components, and other factors by any appropriate method. For example, HS cells can be induced to differentiate in a flat adhesive environment (liquid) or in a 3D adhesive environment (e.g. 1% collagen gel). A microgravity environment can also be used to induce HS cell differentiation, see Ingram et al, In vitro Cell Dev. Biol. Anim., 33(6):459-466 (1997). Yet another method of inducing differentiation is by the generation of stemplasms in immunodeficient mice, Thompson et al., Science, 282(5391):1145-47 (1998), or in other animals. Differentiation is induced in this way by encapsulating HS cells and allowing them to form stemplasms in an appropriate host. For example human HS cells may be encapsulated and placed in the same patient from whom such cells are derived (isogenic), or a different human (allogeneic). Likewise the entire blastocyst-like mass may be implanted into a recipient animal allowing it to form stemplasms.

Currently, a number of techniques are available that allow separation of cells from the immune system of the body using a synthetic, selectively permeable membrane. Such techniques can be used to differentiate HS cells by the generation of stemplasms in vivo. For example, upon implantation of encapsulated HS cells to generate a stemplasm, a membrane can be used to allow free exchange of nutrients, oxygen and biotherapeutic substances between blood or plasma and the encapsulated cells. Such system may modulate the bidirectional diffusion of antigens, cytokines, and other immunological moieties based upon the chemical characteristics of the membrane and matrix support. See Lanza et al., Nat. Biotechnol., 14(9):1107-11(1996). For systems involving implantation of blastocyst-like masses in animals, individual or multiple cell masses may be implanted in a single animal.

HS cells can be produced from any animal donor material and used in any animal system. Both human and non-human HS cells are contemplated by the present invention. Suitable veterinary applications include the generation of HS cells from and use in mammals, fish, reptiles, birds, and amphibians.

The pluripotent isolated HS cells of the present invention can be differentiated into selected tissues for a variety of therapeutic uses including the in vitro culture of differentiated tissues for purposes of study, diagnostics, or for implantation into an individual. Preferably, HS cells will be used therapeutically in the individual that provided the donor material for HS cell formation.

Current techniques used to differentiate pluripotent cells that are known to those skilled in the art include methods for differentiating embryonal carcinoma (EC) cells into a variety of embryonic and extra-embryonic cell types. (See, Andrews, APMIS, 106:158-168 (1998), incorporated by reference herein). Such techniques can also be used to induce differentiation of HS cells. In vitro methods used for directed differentiation of EC cells include exposure of EC cells to various factors known to trigger cell commitment and differentiation into a desired cell type or tissue. Alternatively, the in vitro differentiation scheme employed could involve the removal of growth factors known to favor stem cell maintenance. Upon removal of such factors from the medium, the stem cells form clusters, known as embryoid bodies, within which descendants of all three embryonic germ layers can be found. The presence of certain cell lineages within the embryoid body can then be enhanced through supplementation of the medium with additional growth factors and chemicals. The resulting cell population will then contain an increased proportion of a desired cell type, which then can be selectively isolated. Also see, Edwards et al., Modern Trend, 74(1): 1-7 (2000), incorporated by reference herein, for a discussion of pluripotent stem cells and their use in medicine.

Illustrative examples of differentiation control factors include but are not limited to cytokines, hormones, and cell-regulating factors such as LIF, granulocyte macrophage colony stimulating factor (GM-CSF), IL-3, thyroid hormone (T3), stem cell factor (SCF), fibroblast growth factor (FGF-2), platelet derived growth factor (PDGF), ciliary neurotrophic factor. While stimulating cytokines such as GM-CSF, SCF, and IL-3 have been shown to promote differentiation (see Keil et al., Ann. Hematol., 79(5):243-8 (2000), incorporated by reference herein), inhibitory factors, such as LIF, have been shown to maintain mouse embryonic stem (ES) cells in the undifferentiated pluripotent state (Zandstra et al., Blood, 96(4): 1215-22 (2000), incorporated by reference herein). Further, SCF has been shown to stimulate the differentiation of chicken osteoclasts from their putative progenitors (van't Hof et al., FASEB J., 11(4):287-93 (1997), incorporated by reference herein), while FGF-2 has been shown to play a role both in initiating lactotrope differentiation and maintaining prolactin expression in immortalized GHFT cells, thereby suggesting a mechanism for controlling differentiation of stem cells into different anterior pituitary cells (Lopez-Fernandez et al., J. Biol. Chem. 275(28):21653-60 (2000), incorporated by reference herein). In addition, platelet-derived growth factor (PDGF-AA, -AB, and -BB) supports neuronal differentiation while ciliary neurotrophic factor and thyroid hormone T3 generate clones of astrocytes and oligodendrocytes (Johe et al., Genes. Dev., 10(24):3129-40 (1996), incorporated by reference herein).

Further, WO 01/29206 (Cibelli et al.), published Apr. 26, 2001, describes various differentiation factors, such as differentiation agents, growth factors, hormones and hormone antagonists, extracellular matrix components and antibodies to various factors, and techniques that can be used to induce ES cells to differentiate. Such techniques and reagents/factors can be used in accordance with the present invention, and are hereby incorporated by reference. See also, Schuldiner et al., “Effects of Eight Growth Factors On The Differentiation Of Cells Derived From Human Embryonic Stem Cells,” PNAS 97(21):11307-12 (2000), also incorporated by reference herein.

HS cells may also be induced to differentiate by transplantation in vivo, preferably in situ, where the cells undergo histologic and functional differentiation and form appropriate connections with host cells. Endogenous regulation factors located in the transplant site can direct the differentiation of the stem cell into a particular type of differentiated cell or tissue. Alternatively, groups of divergent differentiated cells and/or tissues result from stem cells transplanted to the hypodermis, the peritoneum, and the renal capsule. See Hogan, supra, pp. 183 to 184, for a detailed description of the kidney capsule implantation procedure.

1. Histological Features and Genotype of Differentiated Cells Found Within Human Teratomas

Teratomas may be composed of mature and/or immature tissues. Morphological analysis of groups of cells comprising several types of differentiated tissue were identified in sections of teratomas affixed to glass slides, and tissue morphology was performed on these teratoma sections using conventional techniques (Zhuang et al., J Pathol, 146:620 (1995), and Vortmeyer et al., Am. J. Pathol., 154:987-991(1999) incorporated by reference herein). Microdissection of teratomas selectively procured individual tissue components including mature squamous epithelium, mature intestinal epithelium, mature cartilage and respiratory epithelium, immature cartilage, mature neuroglial tissue, immature neural tissue, and mature respiratory epithelium. (See Nicolas et al., Cancer Research, 36:4224-4231 (1976), incorporated by reference herein, for a detailed discussion of the variety of cell lines isolated from in vitro transplantable teratocarcinomas and techniques associated therewith.)

After DNA extraction, allelic zygosity was analyzed using multiple genetic markers on several human chromosomes. In an initial study of a limited number of mature tumors, homozygosity of the same allele was consistently detected (Vortmeyer et al., Am. J. Pathol., 154:987-991 (1999), the entire contents of which are hereby incorporated by reference). Analysis of a larger number of teratomas, however, revealed a small number of tumors with loci having heterozygous alleles.

To test the hypothesis that heterozygous teratoma tissue arises from premeiotic cells, ovarian and testicular teratomas containing both mature (differentiated) and immature (undifferentiated) tissue elements were dissected to obtain samples of one variety of mature and immature tissue elements, using the same experimental approach. (See Examples below). Heterozygous alleles were detected in undifferentiated tissue elements including immature squamous epithelium, immature neural tissue and immature cartilage. Differentiated tissue from these tumors was homozygous for the same genetic markers. Differentiated tissue elements tested include mature sebaceous gland tissue and mature squamous epithelium, including duplicate samples taken from separate areas of the same mature element of the same tumor.

The results of this test demonstrate that genetic homozygosity correlates with differentiation into recognizable mature tissue types, and genetic heterozygosity correlates with undifferentiated tissues. Regions of undifferentiated tissue within a teratoma, therefore, are initiated by a teratogenic event in the premeiotic germ cell, while differentiated tissues within teratomas arise from postmeiotic germ cell.

Premeiotic cells contain both copies of each chromosome, such that proliferation of premeiotic cells produces a population of genetically heterozygous cells. In contrast, postmeiotic cells have only one copy of each chromosome and are genetically homozygous. The fact that postmeiotic progenitor cells proliferate to yield mature teratomas, or regions of mature differentiated tissue within a teratoma, suggests that meiosis is not only a mechanism for chromosomal rearrangement and recombination of genetic material, but is also a prerequisite for the activation of specific genes leading to tissue differentiation and development.

Therefore, proliferating tumor cells that have not undergone meiosis will retain undifferentiated, heterozygous characteristics and develop into undifferentiated teratomatous tissue. Differentiated teratomatous tissue may be derived from proliferating teratoma cells that have completed meiosis or may be derived from postmeiotic cells undergoing a teratogenic event.

Thus meiosis is required for tissue differentiation in teratomas. Genetic analysis of tissue elements within teratomas demonstrated that homozygosity is associated with histologically mature differentiated tissues, and genetic heterozygosity is associated with histologically immature, undifferentiated tissues. This result supports the conclusion that meiosis must be complete before teratomatous cells can undergo subsequent tissue differentiation. Thus, the present invention teaches the interruption of germ cell meiosis to create the isolated, undifferentiated, pluripotent homozygous stem cells of the present invention.

2. Differentiation of HS Cells into Specific Cells or Tissues

As noted in the foregoing paragraphs, any method available to those skilled in the art to induce differentiation may be used with the present invention. For example, cells can be cultured in tissue culture wells, each well containing a unique combination of differentiation factors. Nucleic acids or cDNAs encoding such factors can be plated out as naked DNA, as constructs which are prepared to carry such nucleic acids by transfection, or by viruses. Differentiated cells are identified by use of: a) differentiation-specific anti-bodies; 2) morphology; 3) PCR using differentiation-specific primers; or (4 any other applicable technique for identifying specific types of differentiated cells.

Once subjected to the differentiation protocol, primitive cells from a particular cell lineage can be isolated from the differentiated HS cells by conventional techniques. If desired, such isolated differentiated progenitor cells can be expanded by cell culture or other appropriate methods.

Progenitor cells can also be transfected during any appropriate stage of their differentiation. For example, before the formation of the blastocyst-like mass, HS cells may be transfected, and said cells can then be used as nuclear donors for enucleated oocytes. In another embodiment, progenitor cells may be transfected directly after isolation, for example with the CD34+, or CD38 cells of the hematopoietic system.

Any known method for inserting, deleting or modifying a desired gene may be used to produce genetically altered progenitor or HS cells.

Upon implantation of encapsulated HS cells, or blastocyst-like masses, into an animal, teratomas and even teratocarcinomas can be produced. To prevent HS cells from forming benign or malignant tumors in a transplant recipient, genes may be introduced into or deleted from such cells so as to prevent the growth of undifferentiated cells. For example, an inducible promotor such as MMTV can be introduced into cells followed by induction with dexamethasone to drive the expression of a gene that blocks the growth of undifferentiated cells and induces differentiation. Or, a promotor for a gene that is germline specific can be introduced to drive the expression of a cell-cycle blocker or an apoptosis gene.

A preferred method of making differentiated progenitor cells comprises activation of non-fertilized post-meiosis I diploid oocytes using calcium ionophore, and culturing such activated oocytes in culture media to the stage where a blastocyst-like mass is formed. Zona pellucida is then removed from the cell mass using pronase, followed by removal of trophoblastic cells by immunosurgery. With the cell mass remaining, the aggregate of HS cells, is induced to differentiate with or without cytokines using a flat adhesive environment, a 3D adhesive environment, microgravity, generating stemplasms in immunodeficient animals, or isogenic, or allogeneic animals. Differentiated progenitor cells can then be removed from the differentiated cell mass derivatives.

The specific types of cell/tissues that pluripotent cells can be differentiated into are discussed below. However, such discussion is designed to be illustrative not exhaustive. The present invention can be practiced using differentiation methods known in the art, including techniques not recited herein, or not yet discovered.

Differentiation into Endoderm Cell Types

Differentiation of pluripotent cells into various endodermal cell types has great therapeutic implications including use for transplantation purposes, or for enhancing the uptake and processing of nutrients, or to direct pattern formation. HS cells can be induced to differentiate into endodermal progenitor cells by treatment with high doses of RA or by members of the transforming growth factor β superfamily, including bone morphogenetic protein (BMP)-2 (Pera and Herzfeld, Reprod. Fert. Dev., 80:551-555 (1998)). Some HS cell lines can also be induced to differentiate in a distinct, apparently non-neural, direction by hexamethylene bisacetamide (HMBA) (Andrews, APMIS, 106:158-168 (1998)). BMP-2 can be used to specifically trigger differentiation into parietal, or visceral endoderm (Rogers et al., Mol. Bio. Cell, 3:189-196 (1992)). BMPs are molecules that can induce cartilage and bone growth in vivo, but BMP messages are also expressed in many non-bony tissues, including developing heart, hair follicles and central nervous system, indicating a pivotal role in cell commitment and differentiation.

Differentiation into Epithelial Tissues

Epithelial tissues are composed of closely aggregated polyhedral cells with very little intercellular substance. The forms and dimensions of epithelial cells are varied, ranging from high columnar, to cuboidal, to low squamous. Epithelial cell nuclei have a distinctive appearance, varying from spherical to, elongated, to elliptic in shape. Adhesion between these cells tends to be very strong. Thus, cellular sheets are formed that cover the surface of the body and line its cavities. These sheets may take the form of a monolayer, comprised of one type of epithelial cell, or a stratified multilayer, comprised of many different types of epithelial cells.

The principle functions of epithelial cells include: covering and lining (e.g., skin), absorption (e.g., the intestine), secretion (epithelial cells of the glands), sensation (neuroepithelium), and contractility (e.g., myoepithelial cells). An illustrative discussion of the various types of epithelial cells, and of methods for differentiating HS cells to various types of epithelial cells is provided below.

(a) Keratinizing Epithelial Cells

The keratinizing epithelial cells are primarily associated with the epidermal and dermal layers of the body (e.g., hair, skin, nails, etc.). Examples include but are not limited to: keratinocytes of the epidermis and nail bed (differentiating epithelial cells); basal cells of the epidermis and nail bed (epidermal stem cells); and hair shaft (e.g., medullary, cortical, and cuticular), root sheath (e.g., cuticular, Huxley's and Henley's layers, and external) and matrix cells (hair stem cell).

Basal cells are relatively undifferentiated cells in an epithelial sheet that give rise to more specialized cells, which act like stem cells. Basal cells of the squamous epithelium of the skin give rise to keratinocytes of the epidermis and nail bed. Likewise, basal cells of the epithelium of the epididymis (absorptive epithelial cells, discussed below) give rise to epididymal principal cells. Basal cells of the olfactory mucosa give rise to olfactory and sustenacular cells. Thus, basal cells serve as a precursor for more specialized epithelial cells.

Isolated HS cells of the present invention can be differentiated into mature keratinizing epithelial cells, either directly or via suitable precursor cells or basal cells using techniques known in the art for differentiating ES cells, EC cells, other kinds of pluripotent or stem cells, and/or teratocarcinoma cells. See, for example, protocols described by Taylor et al., Cell, 102:451-461 (2000) (the contents of which are incorporated by reference herein) describing the formation of follicles and epidermis from follicular stem cells, particularly bipotent follicular bulge stem cells.

(b) Barrier Epithelial Cells

The barrier epithelial cells can be divided into two classes—wet stratified barrier epithelia and lining epithelia. Wet stratified barrier epithelia include, for example, cells of the urinary epithelium (lining bladder and urinary ducts), and surface and basal epithelial cells of the stratified squamous epithelium of the cornea, tongue, oral cavity, esophagus, anal canal, distal urethra, and vagina (i.e., the cells of the mucosal tissues). Lining epithelia include, for example, cells lining the lung, gut, exocrine glands and urinary tract as well as cells lining closed internal body cavities.

Examples of the epithelial cells lining vessels, ducts, and open cavities include but are not limited to: type I pneumocytes (lining the air space of the lung); pancreatic duct cells (centroacinar cells); nonstriated duct cells of the sweat, salivary and mammary glands; parietal cells and podocytes of the kidney glomerulus; cells of the thin segment of the loop of Henle (kidney); and duct cells of the kidneys, seminal vesicles, prostate, and other glands.

Examples of the epithelia lining closed internal body cavities include but are not limited to: vascular endothelial cells of the blood vessels and lymphatics (fenestrated, continuous, and splenic); synovial cells lining the joint cavities; serosal cells lining the peritoneal, pleural and pericardial cavities; squamous cells lining the perilymphatic space of the ear; cells lining the enolymphatic space of the ear squamous cells; choroid plexus cells (secreting cerebrospinal fluid); squamous cells of the pia-arachnoid; cells of the ciliary epithelium of the eye; and corneal epithelial cells.

Isolated HS cells of the present invention can be differentiated into mature barrier epithelial cells directly, or via suitable precursor cells such as basal cells using techniques known in the art for differentiating ES cells, EC cells, other kinds of pluripotent or stem cells, and/or teratocarcinoma cells. For example, see Wolosin et al., Progress in Retinal and Eye Research, 19(2): 223-255 (2000), incorporated by reference herein, providing a review of stem cells and the differentiation stages of the limbocorneal epithelium.

(c) Exocrine, Endocrine, and Matrix Secreting Epithelial Cells

Exocrine glands secrete products via ducts or canals, onto the free surface of the skin, or onto the free surface of the open cavities of the body, such as the digestive, respiratory or reproductive tracts. Their products are not released into the blood stream. Examples of exocrine products include: mucus polysaccharides and carbohydrates, digestive enzymes, milk, tears, wax, sebum, sweat, seminal fluid and vaginal fluid. Examples of epithelial cells specialized for exocrine secretion include but are not limited to: cells of the salivary gland (mucous and serous); cells of von Ebner's gland in the tongue; cells of the mammary gland; cells of the lacrimal gland; cells of the ceruminous gland of the ear; cells of the eccrine and apocrine sweat glands; cells of the gland of Moll in the eyelid; cells of the sebaceous gland; cells of Bowman's gland in the nose; cells of Brunner's gland in the duodenum; cells of the seminal vesicle gland; cells of the prostate gland; cells of the gland of Littre; cells of the uterine endometrium; isolated goblet cells of the respiratory and digestive tract; mucous cells of the stomach lining; zymogenic and oxyntic cells of the gastric gland; acinar cells of the pancreas; Paneth cells of the small intestine; and type II pneumocytes and Clara cells of the lung.

Isolated HS cells of the present invention can be differentiated into exocrine epithelial cells directly or via suitable precursor cells using techniques known in the art for differentiating ES cells, EC cells, other kinds of pluripotent or stem cells, and/or teratocarcinoma cells. Such techniques are incorporated by reference herein.

Endocrine glands secrete their products, called hormones, directly into the blood stream. Hormones circulate throughout the body to their target areas and act as chemical messengers to regulate specific body functions. Most of the endocrine glands are also epithelial derivatives: they are formed by invagination from an epithelial sheet and initially have ducts connecting them to the free surface of the epithelial sheet. During embryonic development, they lose their ducts and thus are called ductless glands. Their secretory products are released in the interstitial space between cells and diffuse into the blood of the nearest capillaries. Under the microscope, endocrine glands look like any stratified epithelial tissues with one big difference: they do not have a free surface, and are surrounded directly by other tissues.

Examples of endocrines include: oxytocin, vasopressin, serotonin, endorphins, somastatin, secretin, cholecystokinin, insulin, glucagon, bombesin, calcitonin, epinephrine, norepinephrine, steroids, and other hormones. Examples of epithelial cells specialized for endocrine secretion include but are not limited to: cells of the anterior and posterior pituitary; cells of the gut and respiratory tract; cells of the thyroid and parathyroid glands; cells of the adrenal gland; cells of the gonads; and cells of the juxtaglomerular apparatus of the kidney.

Isolated HS cells of the present invention can be differentiated into endocrine secreting epithelial cells, either directly or via suitable precursor cells using techniques known in the art for differentiating ES cells, EC cells, other stem cells and/or teratocarcinoma cells. Such techniques are incorporated by reference herein. For example, Ramiya et al., Nature Medicine, 6(3):278-282 (2000), incorporated by reference in its entirety, describe the in vitro generation of pancreatic islet cells from pancreatic stem cells where Islet producing stem cell (IPSC) cultures were established from digested pancreatic tissue explanted from prediabetic mice. Islet progenitor cells budded from a monolayer of epithelioid-like IPSCs cultured in Earle's high-amino-acid medium with normal mouse serum. Id. VEGF, hepatocyte growth factor, regenerating gene-1, transforming growth factor alpha and islet neogenesis-associated protein were also found to be mitogenic to ductal epithelial cells to give rise to islet endocrine cells. Id. In addition, it was shown that hepatocyte growth factor, beta-cellulin and activin A differentiate acinar cells into insulin-secreting cells. Id. (See also, Serup et al., Nature Genetics, 25:134-135 (2000), Assady et al., Diabetes, 50:1691-7 (2001), and Lumelsky et al., Science, 292: 1389-93 (2001), the contents of which are incorporated by reference herein.)

The major constituent of the connective tissue is its extracellular matrix, which is composed of protein fibers, amorphous ground substance, and tissue fluid. Components of the extracellular matrix are secreted by either the epithelial tissues or connective tissues or both. Examples of epithelial cells specialized for extracellular matrix secretion include but are not limited to: ameloblasts (secreting enamel); planum semilunatum cells of the vestibular apparatus of the ear; and interdental cells of the Corti.

Isolated HS cells of the present invention can be differentiated into extracellular matrix secreting epithelial cells directly or via suitable precursor cells using techniques known in the art for differentiating ES cells, EC cells, other kinds of pluripotent or stem cells, and/or teratocarcinoma cells.

(d) Epithelial Absorptive Cells

Epithelial cells associated with absorption are found in the gut, exocrine glands, and urogenital tract. Examples of such epithelial cells include but are not limited to: brush border cells of the intestine; striated duct cells of the exocrine glands; gall bladder epithelial cells; brush border cells of the proximal tubule of the kidney; distal tubule cells of the kidney; nonciliated cells of the ductulus efferens; and epididymal principle and basal cells.

Isolated HS cells of the present invention can be differentiated into absorptive epithelial cells directly or via suitable precursor cells using techniques known in the art for differentiating ES cells, EC cells, other kinds of pluripotent or stem cells, and/or teratocarcinoma cells.

(e) Contractile Epithelial Cells

Myoepithelial cells are stellate or spindle-shaped cells located between basal lamina and the basal pole of secretory or ductal cells. The function of myoepithelial cells is to contract around the secretory or conducting portion of the gland and thus help propel secretory products toward the exterior. Exemplary myoepithelial cells are found in the iris and exocrine glands.

Isolated HS cells of the present invention can be differentiated into myoepithelial cells directly, or via suitable precursor cells using techniques known in the art for differentiating ES cells, EC cells, other kinds of pluripotent or stem cells, and/or teratocarcinoma cells.

(f) Miscellaneous Epithelial Cells

Certain epithelial cells are particularly specialized for a single function or environment and, as such, cannot be grouped into any of the above categories. For example, lens cells include epithelial cells of the anterior lens, and lens fiber cells. Likewise, pigment cells include retinal pigmented epithelial cells, and melanocytes.

Isolated HS cells of the present invention can be differentiated into specific epithelial cells directly, or via suitable precursor cells using techniques known in the art for differentiating ES cells, EC cells, other kinds of pluripotent or stem cells, and/or teratocarcinoma cells.

Differentiating into Connective Tissues

Connective tissue is characterized by the abundance of intercellular material produced by its cells. The connective tissues are responsible for providing and maintaining form in the body. Functioning in a mechanical role, they provide a matrix that serves to connect and bind the cells and organs and ultimately give support to the body.

Some cells of the connective tissue, such as osteocytes, fibroblasts and adipose tissues, are produced locally and remain there. Other cells come for other territories but circulate and transiently inhabit the connective tissues. The cellular components of connective tissues can be subdivided into the following classes: extracellular matrix secreting cells; cells specialized for metabolism and storage; and circulating cells of the blood and immune systems.

An illustrative discussion of these classes of connective tissue cells is provided below.

(a) Extracellular Matrix Secreting Cells

Examples of extracellular matrix secreting of the connective tissue include but are not limited to: fibroblasts; pericytes of the capillaries; pulposus cells of the intervertebral disc; cementoblasts and cementocytes (secreting bonelike cementum of the root of the tooth); odontoblasts and odontocytes (secreting dentin); chondrocytes (secreting cartilage); osteoblasts and osteocytes; osteoprogenitor cells (osteoblast stem cells); hyalocytes of the vitreous body of the eye; and stellate cells of the perilymphatic space of the ear.

Isolated HS cells of the present invention can be differentiated into extracellular matrix secreting cells, either directly or via suitable precursor cells such as osteoprogenitors, using techniques known in the art for differentiating ES cells, EC cells, other kinds of pluripotent or stem cells, and/or teratocarcinoma cells.

(b) Cells Specialized for Metabolism and Storage

Examples of cells specialized for metabolism and storage include but are not limited to: hepatocytes and adipocytes.

Isolated HS cells of the present invention can be differentiated into cells specialized for metabolism and storage, either directly or via suitable precursor cells using techniques known in the art for differentiating ES cells, EC cells, other kinds of pluripotent or stem cells, and/or teratocarcinoma cells.

In one embodiment, HS cells are induced to differentiate into adipocytes, for example by the method of Dani, Cells Tissues Organs, 165: 173-180 (1999). The capacity of HS cells to undergo adipocyte differentiation invitro provides a promising model for studying early differentiative events in dipogenesis and for identifying regulatory genes involved in the commitment of mesenchymal stem cell to the adipoblast lineage.

A prerequisite for the commitment of HS cells into the adipocyte lineage is to treat HS cell-derived, embryoid bodies at an early stage of their differentiation with retinoic acid (RA) for a short period of time. Two phases are distinguished in the development of adipogenesis from ES cells: the first phase, between day 2 and 5 after embryoid body (EB) formation, corresponds to a permissive period for the commitment of HS cells which is influenced by all-trans-RA. The second phase corresponds to the permissive period for terminal differentiation and requires adipogenesis hormones as previously shown for the differentiation of cells from preadipose clonal lines. The treatment leads to 50-70% of outgrowths containing adipose cells compared to 2-5% in the absence of RA treatment.

RA cannot be substituted by hormones or compounds known to be important for terminal differentiation. Treatment of early EB with insulin, triiodothyronine, dexamethasone or potent activators of PPARs, such as the thiazolidinedione BRL49653, and the nonmetabolizable fatty acid 2-bromopalmitate, alone, or in combination, leads to a low level of adipogenesis (5%). Among factors that have previously been reported to modulate terminal adipocyte differentiation, RA is possibly the only naturally occurring compound able to trigger development of adipose cells from HS cells.

The main function of adipocytes as energy source is to store triglycerides (lipogenic activity) and to release free fatty acids (lipolytic activity) upon hormonal conditions. It can be shown that EB-derived adipocytes display both lipogenic and lipolytic activities in response to insulin and to P-adrenergic agonists, respectively, indicating that mature and functional adipocytes are indeed formed from HS cells in vitro.

PPARs (PPARδ and PPARγ) and C/EBPδ (C/EBPβ, C/EBPδ and C/EBPα) are nuclear factors that regulate genes involved in lipid metabolism. C/EBPα seems to be important to maintain the adipocyte differentiated phenotype, whereas several lines of evidence indicate that PPARγ and C/EBPβ and C/EBPδ are triggers of terminal differentiation of preadipocytes into adipocytes. The role of these factors in the commitment of stem cells into the adipocyte lineage is addressed by studying their expression during the determination and the differentiation periods of HS cells. Expression of PPARγ and C/EBPβ is low during the determination phase and parallels expression of adipocyte-fatty acid binding protein (a-F ABP) which is a marker of terminal differentiation. This result suggests that PPARγ and C/EBPβ are not regulatory genes for the commitment of HS cells into the adipocyte lineage. It has previously been reported that PPARδ gene expression is detected early during rat embryonic development and preceded expression of PPARγ. The same temporal pattern of expression is conserved in developing EBs. In contrast to PPARγ, PPARδ is strongly expressed during the determination phase of HS cells suggesting that this factor could be a good candidate as master gene involved in the commitment of mesenchymal precursors into the adipocyte lineage. However, expression of PPARδ gene is not restricted to adipose tissue and its expression is not modified by the treatment required to induce adipogenesis of HS cells. Stimulation of early EBs by potent activators of PPARδ such as fatty acid 2-bromopalmitate or carbocyclin cannot trigger differentiation of EBs along an adipogenesis pathway.

The generation of HS cells deficient for PPARδ and/or PPARγ will facilitate elucidation of the rule of these transcription factors during the different stages of adipogenesis. Gene targeting via two rounds of homologous recombination generates these mutant HS cells. The differentiation culture system combined with genetic manipulations of undifferentiated HS cells, such as gene trapping and gain or loss of function, should provide a means to identify novel regulatory genes involved in early determinative events in adipogenesis.

Moreover, leukemia inhibitory factor (LIF) and LIF receptor (LIF-R) genes are developmentally regulated during the differentiation of preadipocytes to adipocytes. The fact that LIF and LIF-R are both expressed during the first step of adipocyte differentiation leads to the speculation that this pathway plays a regulatory role in adipogenesis. The role of LIF is addressed by investigating whether LIFR/HS cells are able to undergo adipocyte differentiation. It is known that LIF-null ES cells undergo adipogenesis with comparable efficiency to wild-type cells, which is in agreement with studies of LIF mutant mice indicating that a lack of LIF expression does not prevent the development of adipose tissue. LIF belongs to the IL-6 cytokine family and a feature of members of this family is the redundancy of biological functions.

Therefore, one may postulate that LIF-related cytokines could compensate for the lack of LIF both in vivo and in vitro. The role of LIF-R during adipogenesis is therefore investigated. However, upon generating LIFR/HS cells, it is shown that the capacity of LIF-R null HS cells to undergo adipocyte differentiation is dramatically reduced. Only 5-7% of outgrowths derived from mutant cells contained adipocyte colonies compared to 55-70% of outgrowths derived from wild-type HS cells. The use of genetically modified HS cells combined with conditions of culture to commit stem cells into the adipogenesis pathway facilitates determining the role of LIF-R in the development of adipose cells.

In another embodiment, HS cells of the present invention may be caused to differentiate into hepatocytes using the methods of Hamazaki et al., FEBS Letters, 497:15-19 (2001), that is hereby incorporated by reference.

(c) Circulating Cells of the Blood and Immune Systems

Examples of cells of the blood and immune systems include but are not limited to: red blood cells (erythrocytes); megakaryocytes; macrophages (e.g., monocytes, osteoclasts, Langerhans cells, dendritic cells, and microglial cells); neutrophils; eosinophils; basophils; mast cells; killer cells; T lymphocytes (e.g., helper T cells, suppressor T cells, killer T cells); and B lymphocytes (e.g., IgM, IgG, IgA, IgE, killer cells).

Isolated HS cells of the present invention can be differentiated into circulating cells of the blood and immune systems, either directly, or via suitable progenitor cells such as haematopoietic stem cells, using techniques known in the art for differentiating ES cells, EC cells, other kinds of pluripotent or stem cells and/or teratocarcinoma cells. Such techniques, including those described by Suzuki et al, Int'l J. of Hematology, 73:1-5 (2001) and Cho et al., PNAS, 96:9797-9802 (1999), are incorporated by reference herein.

In one embodiment, HS cells are induced to form hematopoietic lineages. Most, if not all, hematopoietic lineages can be produced following in vitro differentiation of ES cells (Hole, Cells Tissues Organs, 165:181-189 (1999)). Although HS cells will analogously begin to differentiate following the withdrawal of leukemia inhibiting factor (LIF), it appears that the conditions of culture of these pluripotent cell types during that differentiation have a critical role to play in the nature of the cell lineages which are subsequently produced. At least three approaches are to be used: (1) HS cells can be aggregated, and allowed to differentiate in suspension culture; (2) HS cells can be seeded in semisolid culture and allowed to differentiate in situ, and (3) HS cells can be allowed to differentiate in the presence of accessory cell types.

Suspension culture is used based on some of the earliest reports of in vitro differentiation of ES cells. Doetschman et al., Embryol Exp Morphol., 87:27-45 (1985) reported the formation of cystic embryoid bodies from ES cells following the withdrawal of LIF and growth in suspension culture. These bodies contained blood islands (reminiscent of yolk sac hematopoiesis), which were made up of erythrocytes and macrophages. Differentiation in semisolid medium is used based on demonstration by several groups of the production of neutrophil, mast cell, macrophage and erythroid lineages (Wiles and Keller, Development, 111:259-267 (1991); Keller et al., Mol. Cell Biol. 13:473-486 (1993a); Lieschke and Dunn, Exp. Hematol., 23:328-334 (1995). Use of accessory cell lines is contemplated, such as OP9 which have subsequently been demonstrated to show the presence of erythroid, myeloid and lymphoid lineages (Nakano el al., Science, 265:1098-1101 (1994)) the latter including natural killer cell types (Nakayama el al., Blood, 91:2283-2295 (1998)).

Although HS cells can indeed realize the potential to form most, if not all, hematopoietic lineages during differentiation in vitro, it is not so clear as to whether they will do so autonomously. Regarding ES cells, several groups reported the requirement for additional hematopoietic growth factor. The work of Nakano and others (Nakano et al., Science, 265:1098-1101 (1994)) suggests that the use of the macrophage-colony-stimulating factor-deficient cell line OP9 is critical to facilitating comprehensive hematopoietic differentiation. The need for stromal cells is also indicated by investigators the workers using the RP010 stromal cell line; in this case, exogenous growth factors are also used. In contrast, other groups report that commitment to myeloid, erythroid or lymphoid lineages appears to not require exogenous cell lines or growth factors (Hole et al., Blood 90:1266-1276 (1996a)).

For ES cells, apparent differences in outcome of hematopoietic differentiation may be due to several different approaches by these groups. Some have used exogenous cytokines, which may amplify otherwise low levels of specific lineage commitment. Indeed, it is clear that the differentiating ES cells themselves contain transcripts for a wide range of hematopoietic cytokines (Hole et al., Blood, 90:1266-1276 (1996a); Hole et at., Gene Technology, Berlin, Springer, pp 3-10 (1996b)) and factors (Keller et al., Mol. Cell. Biol., 13:473-486 (1993b)), which can influence the commitment process.

Lymphoid progenitors can be produced and isolated following HS cell differentiation in vitro. Adoptive transfer into mice whose lymphoid compartment is compromised by genetic lesion results in ES cell-derived lymphoid repopulation over both the long and short term (Potocnik et al., Immunol. Lett., 57:131-137 (1997)). Early reports suggest that repopulating ability of ES cell-derived hematopoietic progenitors maybe restricted to the lymphoid system, however, further studies show that ES cell-derived cells can demonstrate long-term, multilineage, hematopoietic repopulating potential (Palacios et al., Proc. Natl. Acad. Sci. USA, 92:7530-7534 (1995); Hole et al., Blood, 90:1266-1276, (1996a)).

Long-term repopulating hematopoietic stem cells can be identified following differentiation of ES cells in vitro. By characterizing the time course of this differentiation, ES cells can be used to examine the differential expression of genes at the stage at which hematopoietic stem cells are first emerging as distinct cell type. Hematopoietic stem cells are present within a comparatively brief period of differentiation; multilineage repopulating activity is present at day 4 of differentiation, but not found either at day 3 or day 5. (Hole et al., Blood, 90: 1266-1276 (1996a)). Expression of known hematopoietic genes reinforces the importance of this period in hematopoietic differentiation; expression is dramatically up-regulated in this period (Hole et al., Blood, 90:1266-1276 (1996a); Hole and Graham, Battieres Clin. Hematol., 3:467-483 (1997)). Using a subtractive hybridization approach, Hole and Graham, Battieres Clin. Hematol., 3:467-483 (1997) demonstrated that this model of in vitro differentiation is a rich source of hematopoietic genes; at least two of the novel genes identified are expressed in embryogenesis and hematopoietic cell lines in a manner consistent with early hematopoietic commitment.

Gene trapping can be used to identify genes likely to be involved in early hematopoietic commitment. In this strategy, genes are mutagenized at random by the insertion of a reporter construct into the genome of HS cells, often coupled to an expression construct conferring drug resistance. Typically, the expression profile of the “trapped” gene is then observed following production of chimeric animals; candidate genes can then be identified by sequencing. An alternative approach is to use in vitro differentiation of TS cells as a prescreen. Using the OP9-dependent model of in vitro ES cell hematopoietic differentiation, expression trapping of hematopoietic and endothelial cells has been demonstrated (Stanford et al., Blood 92:4622-4631 (1998)).

In a further embodiment, HS cells are induced to differentiate into lymphocytes. Exemplary protocols using the methods provided by Cho et al., who established an efficient system for the differentiation of ES cells into mature Ig-secreting B lymphocytes (Cho et al., Proc. Natl. Acad. Sci. USA, 96:9797-9802 (1999)), are as follows.

The-BM stromal cell line, OP9, is cultured as a monolayer in αMEM supplemented with 2.2 g/liter sodium bicarbonate and 20% FCS (ES grade and lot tested; Cyclone, Logan, Utah). OP9 media is also used for TS/OP9 co cultures. HS cells are cultured on a confluent monolayer of mitomycin C-treated embryonic fibroblasts with 1 ng/ml leukemia inhibitory factor (R & D Systems, Minneapolis, Minn.). HS and embryonic fibroblast cells are maintained in DMEM, supplemented with 15% FCS, 2 mM glutamine, 110 μg/ml sodium pyruvate, 50 μM2-mercaptoethanol, and 10 mM Hepes (pH 7.4). All co-cultures are incubated at 37° C. in a humidified incubator containing 5% CO2 in air. Periodic testing indicates that all cell lines were maintained as mycoplasma-free cultures.

For hematopoietic induction, a single-cell suspension of HS cells is seeded onto a confluent OP9 monolayer in 6-well plates. The media is changed at day 3; by day 5, nearly 100% of the TS colonies differentiate into mesoderm-like colonies. The cocultures are trypsinized (0.25%; GIBCO/BRL) at day 5; the single-cell suspension is preplated for 30 min; and nonadherent cells (1 to 2×106) are reseeded onto new confluent OP9 layers in 10-cm dishes. At day 6 or day 7, small clusters of hematopoietic-like, smooth round cells begin to appear. At day 8, loosely adherent cells are gently washed off and placed onto new OP9 layers (without trypsin). This treatment enriches cells with hematopoietic potential and leaves behind differentiated mesoderm and undifferentiated HS colonies.

After this passage, hematopoietic colonies expand with noticeable proliferation between days 10 and 12 and thereafter. By day 19, the total number of CD45⁺ cells that are recovered from the HS/OP9 cocultures is approximately 105 cells-Flt-3L is used at a final concentration of 5-20 ng/ml (R & D Systems). Cells are cultured in the presence of exogenous Flt-3L from day 5. The addition of Flt-3L at day 5 appears to represent a temporal window for the enhancement of B lymphopoiesis, because the enhancement is observed when Flt-3L is added at a later time (on or after day 8), The media is changed and/or the cells are passaged without trypsin [i.e., they are made into single-cell suspension and filtered (70 um)] between days 8 and 15.

To generate slgM-B cells, the lympho-hematopoietic cells are harvested at day 15, and replated onto a fresh OP9 monolayer, At day 28, cells are stimulated with lipopolysaccharide (LPS) at 10 /g/ml for 4 days. The cells and culture supernatant are then harvested for flow cytometry and ELISA analysis, respectively. In a separate experiment, cells are stimulated with LPS (100 μg/ml) for 48 hours, and analyzed for the up-regulation of CD80 (B7-1).

To generate transformed cell lines, IL-7 (5 ng/ml) (R & D Systems) is added at day 8 to Flt-3L-containing TS/OP9 co-cultures to maintain immature pre-B Cells-Co-cultures are infected by adding an undiluted virus stock harvested from a 4-day confluent plate of the producer cell line. Co-cultures from a 10-cm dish are infected by replacing the medium with 3 ml of virus stock containing 4 μg/ml of polybrene (Sigma) and IL-7. The plate is rocked periodically at 37° C. for 2 to 4 hours. After this period, ml of fresh OP9 medium containing IL-7 is added to the plate. The medium is changed 5 days later to medium with IL-7, but without Flt-3L. Subsequent media changes lack IL-7. Flow cytometry analyses show that all transformed lines display the same phenotype. In each experiment a significant population of CD45R+ CD24+ IgMe immature pre-B cells are present. Infected cells are grown in bulk, and then cloned by limiting dilution. The presence of integrated copies of the viral genome is confirmed by Southern blot analysis.

Flow cytometric analyses of cells harvested at different times after initiation of the HS/OP9 co-culture reveal that CD45+ cells are first observed by day 5 of co-culture. By day 8, the CD45+ cells also express CD 117 and Sca-1 on their surface, thus displaying a phenotype analogous to that of early hematopoietic stem cells. A significant portion of early hematopoiesis occurring in the coculture system typically gives rise to cells of the erythroid lineage as is evident by the large fraction of CD24+ cells staining positive for TER-119 (days 8 and 12). Although the majority of cocultured day-12 cells belong to the erythroid lineage (CD24^{hi} CD45-TER-119˜), the CD45+ cells express low to high levels of CD45R. This phenotype indicates that B lineage cells emerge from the coculture between days 8 and 12. Although this B lineage phenotype is clearly apparent by day 12, long-term cultures (>20 days) seldom result in the generation of CD¹⁹+IgM B cells.

Flt-3L is added at day 5 of the TS/OP9 co-culture, when hematopoietic cells are first observed. Analysis of the day 19 co-cultures reveals that the addition of Flt-3L dramatically enhances the generation of B lymphocytes from the HS/OP9 co-cultures (60˜/o vs. 6% CD45R+ cells, with Flt-3L and without Flt-3L, respectively). Thus, the addition of Flt-3L to the HS/OP9 co-culture at day 5 increases the recovery of B lineage cells at later times by ˜ten-fold. Significantly, the frequency of myeloid, CD 11 b⁺ (Mac-1), and erythroid, TER-119, cells is diminished in the Flt-3 L-treated cultures. Evidence for T lymphocyte differentiation is not observed in these cultures. The phenotype of day 19 HS/OP9 co-culture cells clearly shows that the addition of Flt-3L results in a specific increase in the generation of CD¹⁹. CD45R-AA4.1-CD²⁴+IgM˜ cells, although one observes only a slight increase in the total number of cells (˜30%). With the addition of Flt-3L at day 5, B lymphopoiesis in the HS/OP9 co-culture system occurs with high efficiency.

The analysis of cells of HS/OP9 co-cultures with Flt-3L that are harvested later show a large increase in the percentage of cells positive for B-lineage markers. After a 4-week culture period, nearly all (>90%) of the cells in the co-culture are B lineage CD45R⁺CD 19+ lymphocytes. These HS-derived B lymphocytes display a CD 11 b⁺ phenotype and a small subset (2 to 3%) of the CD5⁺ B cells, suggesting that CD5⁺ B cells are not generated readily in the HS/OP9 co-cultures.

To demonstrate further the functional capabilities of the in vitro-generated B cells, day-28 cocultures are treated with LPS, after which the mature surface IgM˜CD 19+ B cells increase in size and proliferate extensively. After mitogen activation, one looks for the expression of CD80 (B7-1), a costimulatory molecule that normally up regulates on mature B cells after activation. Furthermore, culture supernatant from LPS-stimulated cells tests positive (by ELISA analysis) for the presence of 119˜, revealing that these cells are capable of robust levels of Ig secretion-

These findings provide evidence for the differentiation of HS cells into mature mitogen-responsive Ig-secreting B cells in vitro.

The addition of exogenous Flt-3L to the HS/OP9 co-culture system is found to be a key element in the development of an efficient and practical model system for the generation of mature, functional B lymphocytes from HS cells in vitro. Various findings support the notion that Flt-3L is an important factor in early B lymphopoiesis in vitro. Moreover, various results elucidate the manner in which the addition of Flt-3L to the HS/OP9 co-cultures facilitates the generation of B lymphocytes. Flow cytometric stages that occur during B cell differentiation in vivo. The fact that HS-derived B cells follow a normal developmental pathway and are functionally analogous to progenitor and mature B cells in vivo leads to the conclusion that this system will prove to be significant in B cell differentiation.

The ability to obtain transformed differentiated stable cell lines from a genetically modified HS cell entirely in vitro will generate additional applications. Because transformants are simple to produce and maintain and have a rapid doubling time, the derivation of cell lines will add to the armory of possible approaches in studying lineage-specific gene-targeted mutations. For example, null mutations in certain genes involved in V(D)J recombination can be assessed.

The present invention contemplates a system for the generation of human B cell progenitors and/or B lymphocytes directly from HS cells in vitro. Such a system would provide a limitless source of genetically defined HS cell-derived B cells with therapeutic applications for individuals suffering from agammaglobulinemias or specific B cell dysfunctions.

Differentiating into Muscle Tissues

Muscle tissue is composed of elongated cells having the specialized function of contraction or propulsion (e.g., ciliated cells).

Examples of contractile cells include but are not limited to: skeletal muscle cells (red; white, intermediate, spindle and satellite); heart muscle (ordinary, nodal and Purkinje fiber); and smooth muscle.

Satellite cells are muscle stem cells involved in the regeneration of skeletal muscles. These cells are mononucleated spindle-shaped cells that lie within the basal lamina surrounding each mature muscle fiber. They are considered to be inactive myoblasts that persist after muscle differentiation. However, following appropriate stimuli, these normally quiescent cells become activated, proliferating to form new skeletal muscle fibers.

Myoblasts are post-mitotic cells capable of fusing together to give rise to myotubes that eventually develop into skeletal muscle fibers. Thus, myoblasts are recognized as the immediate precursors of skeletal muscle fibers.

Isolated HS cells of the present invention can be differentiated into contractile muscle cells directly, or via suitable precursor cells such as satellite cells or myoblasts, using techniques known in the art for differentiating ES cells, EC cells, other kinds of pluripotent or stem cells, and/or teratocarcinoma cells. Such techniques include procedures such as those described by McKarney et al, Int J. Dev. Biol. 41(3):385-90 (1997), and Gussoni et al., Nature 401(6751):390-4 (1999), the contents of which are incorporated by reference herein. McKarney et al. describe the effect of myogenic regulatory factors (myf5, myogenin, MyoD1 and myf6) on embryonic stem cell differentiation. Gussoni et al., describe the delivery of normal hematopoietic stem cells into irradiated animals, resulting in the reconstitution of the hematopoietic compartment of the transplanted recipients.

Examples of ciliated cells with propulsive function include but are not limited to: ciliated cells of the respiratory duct; ciliated cells of the oviduct and endometrium; ciliated cells of the rete testis and ductulus efferens; and ciliated cells of the central nervous system.

Moreover, adipocytes and skeletal myocytes are believed to be derived from the same mesenchymal stem cell precursor and it has been suggested that in vitro the skeletal muscle and adipose development programs are mutually exclusive. In vitro, there is often an inverse relationship between skeletal muscle and adipose tissue. In contrast to the adipocyte lineage, the skeletal myocyte lineage appears. Single EB pretreated with a low development spontaneously during differentiation of HS cells concentration of RA (10⁻⁸ M) can give rise subsequently to both adipocytes and skeletal myocytes (determined by expression of a-FABP and myogenin genes, respectively). However, as the concentration of RA is increased, a shift in the progression of the differentiation program occurs. At an RA concentration higher than 10⁻⁸ M, the expression of myogenin is inhibited and expression of a-F ABP is increased.

Expression of a-F ABP and myogenin genes is paralleled by the development of adipocytes and myocytes scored by microscopic examination. Expression of the A₂COL₆ gene, which is mainly expressed by esenchymal cells, is not modified suggesting that pretreatment of early EBs with RA does not lead to generalized changes in the development program of HS cells. A switch from myogenesis to adipogenesis can be induced by RA in a concentration-dependent manner. Although studies of expression of early gene markers of skeletal myogenesis, such as Myf 5 or MyoD, are required to know at which stage the development of myoblast precursors is blocked, these results lead to the conclusion that the permissive period for the commitment of HS cells into the adipocyte lineage is also critical for the myocyte lineage. In vitro differentiation of HS cells can allow characterization of factors involved in the decision of stem cells to follow the adipogenesis or myogenic developmental pathway.

Isolated HS cells, and progenitor cells of the present invention may also be induced to differentiate into cardiomyocytes using techniques known in the art such as Kehat et al., J. Clin. Invest., 108:407-414 (2001) and Muller et al., The FASEB Journal, 14: 2540-2548 (2000), that are incorporated by reference herein.

Differentiation into Nervous Tissues

Nervous and sensory tissue is composed of cells with elongated processes extending from the cell body that have the specialized functions of receiving, generating, and transmitting nerve impulses. Cells of the nervous and sensory tissues fall into four classes: autonomic neurons; neurons and glial cells; supporting cells of the sense organs and peripheral neurons; and sensory transducers. An illustrative discussion of the various classes of nervous and sensory cells is provided below.

The isolated HS cells and progenitor cells of the present invention can be induced to different into the various kinds of nervous tissue using techniques known in the art, including Guan et al., Cell Tissue Res, 305:171-176 (2001), Przyborski et al., Eur. J. of Neuroscience, 12:3521-28 (2000), Brustle et al., Science, 285: 754-6 (1999), Hancock et al., Biochem. & Biophys. Res. Comm., 271:418-421 (2000), Liu et al., PNAS, 97(11): 6126-31 (2000), and Fairchild et al., Curr. Bio., 10(23): 1515-18 (2000), the contents of which are incorporated by reference herein.

(a) Differential Activation of Homeobox Genes by Retinoic Acid

Homeobox genes, which specify positional information in Drosophila and vertebrate embryogenesis, are responsive to RA, which is a natural morphogen. In human HS cells, RA can be used to specifically activate the expression of all of the four clusters of human Antennapedia-like homeobox genes, known as HOX1, 2, 3, and 4. See, for example, Bottero et al., Rec. Res. Cancer Res. 123:133-143 (1991), incorporated by reference herein, demonstrating that human HOX2 genes are differentially activated in EC cells by RA in a concentration-dependent fashion and in a sequential order co-linear with their 3′ to 5′ arrangement in the cluster.

These genes are normally expressed along the anterior-posterior axis of the developing central nervous system, where 3′ genes are expressed more rostral in the myencephalon, and 5′ genes more caudally in the spinal cord. The concentration dependence of homeobox genes means that HS cells can be exposed to a particular concentration of RA to elicit expression of a particular homeobox cluster or an individual gene within a cluster, thus eliciting commitment to differentiation into tissue of the type corresponding to a precise location, e.g., corresponding to a subregion of the central nervous system.

(b) Autonomic Neurons

Examples of autonomic neurons include but are not limited to: 1) cholinergic neurons; 2) adrenergic neurons; and 3) peptidergic neurons.

Isolated HS cells of the present invention can be differentiated into autonomic neurons, directly, or via suitable precursor cells using techniques known in the art for differentiating ES cells, EC cells, other kinds of pluripotent or stem cells, and/or teratocarcinoma cells.

(c) Neurons and Glial Cells

Examples of neurons and glial cells include but are not limited to: 1) neurons, 2) astrocytes, and 3) oligodendrocytes.

Isolated HS cells of the present invention can be differentiated into neurons and glial cells, either directly, or via suitable intermediate cells such as neuronal precursor cells, using techniques known in the art for differentiating ES cells, EC cells, other kinds of pluripotent or stem cells, and/or teratocarcinoma cells. Such techniques include procedures such as those described below, the entire contents of which are hereby incorporated by reference.

Woodbury et al., J. Neurosci. Res. 61:364-370 (2000), incorporated by reference herein) describe the use of serum free medium containing 1-10 mM BME (SFM/BME) to induce neuronal phenotype in recombinant marrow stromal cells (rMSCs).

Lee et al., (Nature Biotech. 18:675-678 (2000), incorporated by reference herein) describe the efficient generation of midbrain and hindbrain neurons, particularly dopaminergic and serotonergic neurons, from mouse embryonic stem cells using mitogen and specific signaling molecules and factors such as sonic hedgehog (SHH), FGF-8, ascorbic acid cAMP and analogs thereof.

Okabe et al., Mech. Dev. 59: 89-102 (1996), incorporated by reference herein) describe culture conditions that allow the differentiation of neuroepithelial precursor cells from embryonic stem (ES) cells. Specifically, a highly enriched population of neuroepithelial precursor cells derived from ES cells proliferates in the presence of basic fibroblast growth factor (bFGF). These cells differentiate into both neurons and glia following withdrawal of bFGF.

McDonald et al., Nature Medicine 5:1410-1412 (1999), incorporated by reference herein) describe in situ transplantation as a means of directing differentiation. Specifically, neural differentiated mouse embryonic stem cells were transplanted into a rat spinal cord, 9 days after traumatic injury. Histological analysis 2-5 weeks later showed that transplant-derived cells survived and differentiated into astrocytes, oligodendrocytes and neurons, and migrated as far as 8 mm away from the lesion edge.

Borlongan et al., NeuroReport 9:3703-3709 (1998), incorporated by reference herein) describe use of retinoic acid to stimulate the development of post-mitotic neuron like (hNT) cells from immortal human embryonal carcinoma cells (Ntera2 or NT2 cells).

Oliver Brüstle et al., “Protocol for Producing Glial Precursors from pES Cells: A Source of Myelinating Transplants”, The American Association for the Advancement of Science 285: 754-756 (1999), further describe techniques for differentiating ES cells into glial cells. Such techniques can be used for differentiating HS cells, and are incorporated by reference herein.

To characterize electrophysiological properties of neuronal precursor cells, they can be maintained in neurobasal medium containing B27 and 5% FCS for more than 12 days, and the activity of 15 cells is recorded from three plates. The resting membrane potential of such cells should be about −60 mV, and they should exhibit inward action current upon depolarization by 20 mV from the resting potential.

Inward currents are followed by a fast inactivating outward current (I_{A}) and a sustained outward current. These currents should be absent in Cs-filled cells indicating that they are likely to be mediated by outward K-rectifying channels.

Most neuronal cells express spontaneous synaptic currents of varying durations and magnitudes. Application of glutamate onto cells adjacent to the recorded neuronal precursor cell, i.e. putative neurons, can trigger the generation of these synaptic currents in the recorded neuron with a short delay. The recorded synaptic currents are of two types: fast excitatory postsynaptic currents, reversing at about 0 mV, and slow-decaying inhibitory synaptic current, reversing at about −50 mV when recorded in acetate-containing pipettes. Moreover, recorded cells should also respond to topical application of glutamate with a marked inward current recorded at resting potential. The spontaneous and evoked synaptic responses, as well as the responses of the cells to glutamate indicate that recorded cells in culture maintain an array of properties akin to those of prenatal, cultured CNS neurons.

Stimulation of recorded cell with NMDA induces the phosphorylation of the cyclic adenosine monophosphate response element binding protein (CREB protein) and transcription of the c-fos gene. These two inductions are analyzed to determine whether functional NMDA receptors are expressed. Unstimulated cells should not stain with phospho-CREB. In contrast, recorded neuronal cells after stimulation with either glutamate or NMDA for 10 min should show intense nuclear immunoreactivity, and stain with phospho-CREB. In contrast, the large nuclei of glia-like cells should show no phospho-CREB staining.

Further, RT-PCR of cells treated with glutamate, or NMDA reveals c-fos induction suggesting that some of the neurons in this preparation have functional NMDA receptors. The presence of synaptic connections can also be confirmed by electron microscopy, or by morphological characteristic, for example, typical pre-synaptic structures containing numerous synaptic vesicles should be observed, or thickening of the membrane, which is characteristic of the active zone. Such results suggest that neuronal precursor cells derived from HS cells can be differentiated into post-mitotic neurons, which form functional synaptic connections.

Previous studies have shown that bFGF is a strong mitogen for neuroepithelial precursor cells. To investigate the response of HS cells to bFGF, HS cells kept in ITS/FN medium for 6-7 days are dissociated and plated in several different DMEM/F12-based media. Three days later cell density is measured. A combination of DMEM/F12 medium supplemented with modified N3 (mN3) medium, bFGF and fibronectin should have the highest proliferative effect. At concentrations of 5 to 50 ng/ml, bFGF should show the same effect on proliferation. At concentrations lower than 1 ng/ml, bFGF should not show clear proliferative effects. Since laminin is expected to show a slightly higher stimulation of cell proliferation than fibronectin, a combination of N3 medium, bFGF and laminin (“N3FL” medium) is used as a proliferation condition for neuronal precursor-like cells.

In N3FL medium, the predominant proliferating cells should resemble ITS/FN medium-induced nestin-positive cells. When grown in N3FL medium, HS cells like various ES cell lines (D3, CJ7 and J1) should take on the same morphology, and their proliferation should be strictly dependent on bFGF. Cell proliferation is quantified by counting the cell density 1, 4 and 7 days after plating. Cell counting should show a six-fold increase in cell number after 7 days in culture.

It is also possible to stain the preparation with antibodies specific to neuronal precursors (nestin), post-mitotic neurons (microtubule-associated protein 2; MAP2), astrocytes (glial fibrillary acidic protein; GFAP) and oligodendrocyte-lineage cells (O4, Gal-C). Nestin-positive cells should be greater than 80% of the total cell population at each time point; MAP2-positive cells should be about 10-15% of the total cell population; and GFAP-positive cells should be less than 2% of the total cell population. There should be no O4- or Gal-C-positive cells observed in this preparation.

Moreover, neural progenitors isolated from the adult central nervous system differentiate into neurons and glial cells after transplantation into brain, and differentiate into oligodendrocytes and astrocytes after transplantation into spinal cord. Similarly, stem cells can be transplanted into the spinal cord where they undergo differentiation and migration, and promote recovery in injured spinal cords. McDonald et al., 1999, Nature Medicine 5:1410-1412, transplanted ES cells that have been exposed to RA (retinoic acid) to induce neural differentiation (4 day exposure to 500 nM all-trans-RA) and observed differentiation into astrocytes, oligodendrocytes and neurons, migration within the spinal cord, and behavioral (locomotor) outcomes indicating recovery in injured spinal cords.

In one embodiment, HS cells can be substituted for ES cells and transplanted into the spinal cord to undergo differentiation and migration, and promote recovery in a patient in need of such therapy. HS cell derived embryoid bodies (4 days without, then 4 days with retinoic acid) are used for transplantation, where RA is used to induce neural differentiation. Partially trypsinized embryoid bodies are transplanted as cell aggregates into the syrinx that forms 9 days after spinal cord contusion. Sham-operated controls are handled identically, but in place of cell transplantation they receive intra-syrinx injections of culture medium only. Motor function is assessed using the Basso-Beattie-Bresnahan (BBB) Locomotor Rating Scale.

The day before transplantation (day 8 after injury) BBB scores are obtained, control and experimental groups are matched, and subjects are assigned randomly to groups, to ensure that initial locomotor scores are equalized between groups. At day nine (9) after impact injury, subjects receive transplants of neural differentiated HS cells (approximately 1×10⁶) or vehicle medium by means of a spinal stereotaxic frame, a glass pipette with a tip 100 μm in diameter configured to a 5-μl Hamilton syringe, or a Kopf microstereotaxic injection system (Kopf Model 5000 & 900; Kopf, Tujunga, Calif.). The HS cell or vehicle medium (5 μl) is injected into the center of the syrinx at the T9 level over a 5-minute period. Three independent experiments, with time-matched controls, are completed in total. The first series is completed for behavioral analysis and late histologic analysis (n=11 per group) 5 weeks after transplantation and HS cell transplantation is compared with the control. The second series is used to compare early (2 weeks after transplantation) and late (5 weeks after transplantation) histological outcomes (n=11 per group) and HS cell transplantation (ROSA lacZ transgene line) compared with the control.

HS cell-derived cells marked genetically and pre-labeled in vitro with a 24-hour pulse of 10 uM BrdU are identified in situ 14-33 days after being transplanted. Identification can also be achieved with specific antibodies. At 2-5 weeks after transplantation, HS cell-derived cells should be found in aggregates or dispersed singly throughout the injury site. Furthermore, single cells should be found as far as 8 mm away from the syrinx edge in either the rostral or caudal direction. In most of the transplanted subjects, by 2 weeks after transplantation, HS cell-derived cells should fill the space normally occupied by a syrinx in medium-treated subjects. By 5 weeks, the density of HS cell-derived cells in this area should be reduced and replaced with an extracellular matrix containing fibers.

Surviving HS cell-derived cells can be identified with antibodies against markers specific for oligodendrocytes (adenomatous polyposis coli gene product), astrocytes (glial fibrillary acidic protein), and neurons (neuron-specific nuclear protein). Nuclei can be identified distinctly with Hoechst 33342 staining. Most surviving HS cell-derived cells should be oligodendrocytes and astrocytes, but some HS cell-derived neurons should also be present in the middle of the cord. Many of the HS cell-derived oligodendrocytes should also be immunoreactive for myelin basic protein, an integral component of myelin.

Performance in “open field locomotion” is enhanced by HS cell transplantation. In contrast to the inability of the sham-operated transplantation group to support weight, subjects transplanted with HS cells should demonstrate partial weight-supported ambulation. A statistical difference in BBB scores should be achieved by 2 weeks after transplantation. After 1 month, there is should be a difference of two points on the BBB scale between the sham-operated and HS cell transplantation groups. The score obtained by the former indicates no weight-bearing and no coordinated movements, whereas the latter score indicates a gait characterized by partial limb weight-bearing and partial limb coordination.

In summary, HS cell-derived cells when transplanted into the spinal cord 9 days after weight-drop injury should survive for at least 5 weeks; migrate at least 8 mm away from the site of transplantation; differentiate into astrocytes, oligodendrocytes and neurons without forming tumors; and produce improved locomotor function.

Neuronal cells previously induced to differentiate by the withdrawal of bFGF can be maintained without significant cell death in neurobasal medium plus B27 supplement and 5% fetal calf serum for more than 2 weeks. This long-term culture may successfully be applied to J1, CJ7 and D3 cell lines. Long-term culture is difficult, however in N3-based serum-free medium.

Double labeling of cells in culture with MAP2 and neurofilament-M (NF-M) should indicate that two classes of neurites are present. Anti-MAP2 antibody stains short thick processes and cell bodies while anti-NF-M stains thin, long processes. HS cell-derived neurons upon double labeling should have MAP2-positive dendrites and NF-M-positive axons.

Staining with anti-synapsin I reveals punctuate structures closely associated with the plasmalemma of dendrites. Such staining pattern should indicate the segregation of synaptic vesicles to distinct sites along the axons.

To investigate neurotransmitter phenotypes, HS cells that are differentiated into neuronal cells are stained with several antibodies against neurotransmitters. Results should indicate large numbers of glutamate-positive cells mixed with completely negative cells.

Further, gamma-aminobutyric acid (GABA)-positive cells are common, and GABA staining is also available. Therefore, it is possible to identify thin processes that are GABA-positive but MAP2-negative. This finding suggests the differentiation of the dendritic and axonal structures, since the axons of GABA-nergic neurons should be GABA-positive and MAP2-negative.

Neuronal gene expression can be further analyzed by reverse transcription-polymerase chain reaction (RT-PCR) using a panel of neuron-specific primers. The preparation contains cells expressing glutamate decarboxylase (GAD65)′ calbindin D₂₈, NMDA receptors 1, 2A. 2B, 20, (1-amino-3-hydroxy-5-methylisoxazole-4-propionate (AMPA) receptors, and GABA_{A} receptor. In every case, much higher amounts of transcripts should be detected in total RNA from differentiated cells.

To investigate whether these neuronal cells correspond to cells at any specific CNS regions, expression of three position-specific markers along the anterior-posterior axis are analyzed. Otx-1 is mainly expressed in forebrain and midbrain, En-1 in the midbrain-hindbrain boundary, and Hoxa-7 in the posterior spinal cord. Undifferentiated HS cells should express Hoxa-7, but not Otx-1 and En-1 expression. Therefore, the expression of the posterior marker Hoxa-7 should be down-regulated in nestin-positive cells proliferating in the presence of bFGF for more than 10 days. In contrast, Otx-1 and En-1 should be up-regulated in these proliferating cells. After differentiation by switching to neurobasal medium containing B27 and serum, Hoxa-7 expression should be up-regulated again, and Otx-1 and En-1 expression should be maintained. The presence of different transcriptional factors suggests that the preparation generates neurons characteristic of different CNS regions.

(d) Supporting Cells of the Sense Organs and Peripheral Neurons

Examples of supporting cells of the sense organs and peripheral neurons include but are not limited to: supporting cells of the organ of Corti (e.g., inner and outer pillar cell, inner and outer phalangeal cell, border cells, Hensen cells); supporting cells of the vestibular apparatus; supporting cells of the taste buds; supporting cells of the olfactory epithelium; Schwann cells; enteric glial cells; and satellite cells.

Isolated HS cells of the present invention can be differentiated into such supporting cells directly or via suitable precursor cells using techniques known in the art for differentiating ES cells, EC cells, other kinds of pluripotent or stem cells, and/or teratocarcinoma cells.

(e) Sensory Transducers

Examples of sensory transducers include but are not limited to: 1) photoreceptors; hearing sensors (e.g., inner and outer hair cell of Corti); 2) acceleration and gravity sensors; 2) taste sensors (type II taste bud cell); 3) smell sensors (e.g., olfactory neurons); blood pH sensors (carotid body cell, type I, type II); 4) touch sensors (e.g., Merkel cell of the epidermis, primary sensory neurons); 5) temperature and pain sensors (e.g., primary sensory neurons); and 6) configurations and forces sensor in the musculoskeletal system (proprioceptive primary sensory neurons).

Isolated HS cells of the present invention can be differentiated into sensory transducers, particularly primary sensory neuron, either directly, or via suitable precursor cells such as basal cells, using techniques known in the art for differentiating ES cells, EC cells, other kinds of pluripotent or stem cells, and/or teratocarcinoma cells.

Differentiating into Reproductive Cells

Cell involved in reproduction include germ cells, such as oocytes and spermatocytes, and nurse cells, such as ovarian follicle cells, thymus epithelial cells, and Sertoli cells.

Isolated HS cells of the present invention can be differentiated into reproductive cells, either directly or via suitable precursor cells such as ooginium, spermatogonium or primordial germ cells (originating in the endoderm of the yolk sac), using routine experimentation and conventional techniques such as those known in the art for differentiating ES cells, EC cells, other kinds of pluripotent or stem cells, and/or teratocarcinoma cells.

### E. EXAMPLES

### Example 1

### Homozygous Stem Cell Formation, and Their Differentiation into Progenitor Cells and Various Tissues of the Three Embryonic Germ Layers Within Teratomas

### Example 1(a)

### Derivation of HS Cells from Mouse Post-Meiosis I Oocytes by Activation Followed by Prevention of the Extrusion of the Secondary Polar Body

73 Oocytes were obtained from hybrid (BDA2 F1: C57 black×DBA2, Charles River Laboratories, Wilmington, Mass.), eight-week old, female mice by superovulation using the following procedure. Three hybrid mice were administered injections of 5 IU/100 ul of pregnant mare's serum gonadotropin (PMS; PCCA, Houston, Tex. (29-1000-IBX)), and 5 IU/100 μl of human chorionic gonadotropin (HCG; Sigma, St. Louis, Mo., (C8554)) about 48 hours apart.

Oocytes were harvested about 17 hours after the HCG injection, and the cumulus mass was removed without allowing a resting period by incubating the freshly obtained oocytes in a drop (˜300 μl) of hyaluronidase (H4272, Sigma) dissolved in M2 media (M7167, Sigma) at final concentration of 0.3 mg/ml. Oocytes were then washed three times with HEPES buffered M2 media before further handling.

Oocytes were then activated by treatment with 5 μM calcium ionophore (C7522, Sigma: Ca²⁺ 1 mg/764.8 ul; DMSO 2.5 mM=500×(stock); final concentration 2 ul Ca²⁺ (500×)/1 ml M₂=5 μM) solution at room temperature for five minutes. Oocytes were then washed twice with HEPES buffered M2 media.

Ca++ activated oocytes were incubated in M16 bicarbonate-buffered culture media (M7292, Sigma) containing 6-dimethylaminopurine (6-DMAP (D2629, Sigma): 250 mM=50×(stock); final concentration: 20 μl/1 ml M₁₆=5 mM), and 5% CO₂ at 37° C. for 3 hours. Oocytes were then washed three times with M16 media and incubated in a drop of M16 media under mineral oil for at least 4 days.

After 4-5 days incubation in M16 media, cell masses resembling blastocysts were obtained from Ca++ activated oocytes. After the shell surrounding these blastocyst-like masses detaches (“hatching”), they were transferred on to a mitomycin-C treated murine embryonic feeder cell layer for at least 15 days in ES medium (DMEM: Gibco, Life Technologies, Rockville, Md. (11995-065); 20% FBS: Gibco (16141-079)) for stem cell formation.

Alternatively, stem cells were derived from hatched blastocyst-like masses by immunosurgery. Hatched blastocyst-like masses were incubated with anti-mouse Thy-i rabbit serum (1:10, ACL2001, Accurate Chemical, Westbury, N.Y.) and anti-human lymphocytes rabbit serum (1:10, CL8010, Accurate Chemical) for one hour at 37° C. The cell masses were washed three times with M2 medium and incubated with guinea pig complement (1:10, ACL4051, Accurate Chemical) for 30 minutes at 37° C. to lyse trophoblastic cells. Complement-treated cell masses were then washed 3 times in the M2 medium and transferred to a mitomycin-C treated murine embryonic feeder cell layer for stem cell formation for at least 15 days.

Murine embryonic fibroblasts feeder cells were purchased from Stemcell, Inc. (00308), and passaged 2-3 times. One 60 mm dish of confluent-expanded feeder cells was treated with 5 ml of DMEM/10% FBS medium containing mitomycin-C (final concentration: 10 μg/ml, Sigma M4287) at 37° C. for three hours. Treated feeder cells were then washed with 5 ml DMEM/10% FBS three times, and collected by 1 ml trypsinization at 37° C. for 5 minutes, neutralization with 5 ml DMEM/10% FBS medium, and centrifugation at 1000 rpm for 5 minutes. The mitomycin-treated cell pellet obtained was resuspended in 15 ml DMEM/10% FBS medium, plated on three 60 mm dishes (5 ml of cell suspension/dish), and incubated at 37° C. overnight before use.

### Example 1(b)

### Development of Blastocyst-Like Cell Masses from Human Diploid Oocytes by Activation Followed by the Prevention of the Extrusion of the Secondary Polar Body

Female ovum donors underwent down-regulation with leurpolide acetate (Lupron: TAP Pharmaceuticals, Deerfield, Ill.) and then began COH (Controlled Ovarian hyper-stimulation) by receiving follicle stimulating hormone (FSH) (Seromo Pharmaceutical) treatment at a dosage of 300 IU/day to induce an appropriate multifollicular response. When ultrasonographic criteria for follicular maturity were met, a single 10,000 IU dose of hCG was administered, and transvaginal follicular aspiration was performed approximately 36 hours after hCG administration. Cumulus from retrieved oocytes were removed after a resting period of about 1½ hours by exposing them to 80 IU/ml hyaluronidase for approximately 30 seconds followed by HEPES-buffered human tubal fluid supplemented with 10% human serum albumin (InVitroCare, Inc., San Diego, Calif.).

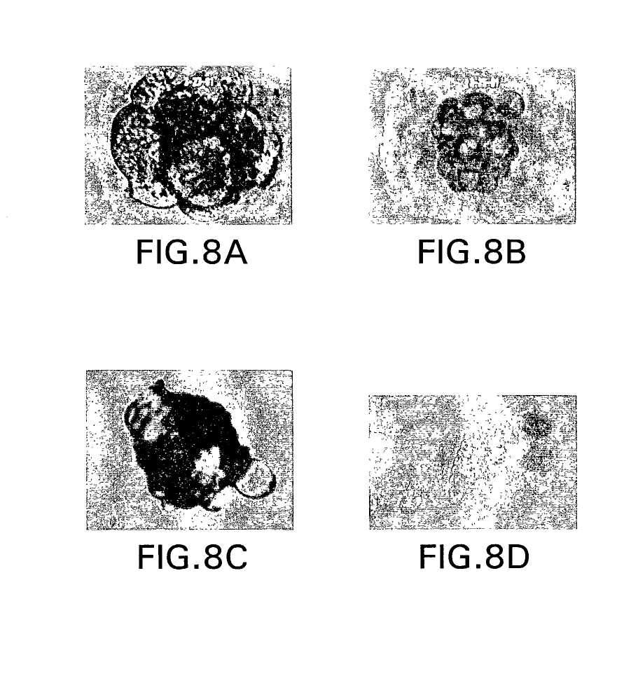

To accomplish mitotic activation, the cumulus free mature M-II oocytes were treated with 5 μM calcium ionophore (A23187, Sigma) for 5 minutes at 33° C. followed by incubation in 1 to 5 mM 6-dimethylaminopurine (6-DMAP, Sigma) for 3 to 5 hours at 37° C. The activated oocytes were incubated in IVC-1 medium (InVitroCare, Inc.) for 3 days, and further incubated in IVC-3 (InVitroCare, Inc.) for 2 days for cell division and blastocyst formation. Alternatively, day number 2 embryo like cell masses can be cocultures on STO feeder cells. On day 6 assisted hatching was performed under a micromanipulator by applying acidified tyrodes solution to about 1/8 of the total exterior surface area of the zona pellucida, using one arm of the micromanipulator, while securing the zone pellucida with the other arm. The blastocyst was then released from the weakened zona, and were then treated with anti-Thy1 and complements to immunosurgically eliminate cells in trophectorderm. Treated blastocysts were then cultured on mitomycin treated STO feeder cells (ATCC) in stem cell culture medium containing 20% fetal bovine serum (Life technologies) in DMEM medium supplemented with nonessential amino acid, pen-strep (Life Technologies), beta-mercaptoethanol (Sigma), and LIF (Chemicon). See FIG. 8D.

### Example 1(c)

### Development of Blastocyst-Like Cell Masses from Human Post Meiosis I Diploid Oocytes by Activation Followed by Allowing the Extrusion of the Secondary Polar Body and Genomic Self-Replication

Female ovum donors underwent down-regulation with leuprolide acetate (Lupron: TAP Pharmaceuticals, Deerfield, Ill.), and then began COH (Controlled Ovarian Hyper-stimulation) by receiving follicle stimulating hormone (FSH) (Serono Pharmaceuticals) treatment at a dosage of 300 IU/day to induce an appropriate multifollicular response. When ultrasonographic criteria for follicular maturity were met, a single 10,000 IU dose of hCG was administered, and transvaginal follicular aspiration was performed approximately 36 hours after hCG administration. Cumulus from retrieved oocytes were removed after a resting period of about 1½ hours by exposing them to 80 IU/ml hyaluronidase for approximately 30 seconds followed by HEPES-buffered human tubal fluid supplemented with 10% humans serum albumin (InVitroCare, Inc., San Diego, Calif.).

To accomplish mitotic activation, the cumulus free mature M-II oocytes were subjected to sham ICSI (intracytoplasmic sperm injection) to mimic activation introduced by sperm followed by incubation with 25 μM calcium iononphore (A23187, Sigma) for 5 minutes at 33° C. Oocytes activated in this manner extrude the secondary polar body and become haploid. Such haploid oocytes were incubated in IVC-1 medium (InVitroCare, Inc.) for 3 days, and further incubated in IVC-3 (InVitroCare) for 2 days for cell division and blastocyst formation. Alternatively, day number 2 embryo like cell masses can be co-cultures on STO feeder cells. On the sixth day, assisted hatching was performed under a micromanipulator by applying acidified tyrodes to the exterior of zona of a blastocyst. The blastocysts were then released from the weakened zona, and cultured on mitomycin-treated STO feeder cells (ATCC) in stem cell culture medium containing 20% fetal bovine serum (Life technologies) in DMEM medium supplemented with nonessential amino acid, pen-strep (Life Technologies), beta-mercaptoethanol (Sigma), and LIF (Chemicon).

Haploid oocytes resulting from activation are able to self-replicate their genome without cytokinesis and give rise to diploid cells (Taylor, A. S., et al., “The early development and DNA content of activated human oocytes and parthenogenetic human embryos,” Hum. Reprod. 9(12):2389-97 (1994); Kaufman, M. H. et al., “Establishment of pluripotential cell lines from haploid mouse embryos,” J. Embryol. Exp. Morphol. 73:249-61 (1983). On day 6 assisted hatching was performed under a micromanipulator by applying acidified tyrodes to the exterior of zona of a blastocyst. The blastocysts were then released from the weakened zona, and cultured on mitomycin treated STO feeder cells (ATCC) in stem cell culture medium containing 20% fetal bovine serum (Life technologies) in DMEM medium supplemented with non-essential amino acid, pen-strep (Life Technologies), beta-mercaptoethanol (Sigma), and LIF (Chemicon). See FIGS. 8A-C.

### Example 1(d)

### Mouse HS Cell Growth, Differentiation of Such HS Cells Under Mouse Kidney Capsule, and Embryoid Body Formation of Such Cells

HS cells obtained from blastocyst-like masses as described in Example 1(a) were seeded on 0.1% gelatin coated dishes (10 cm) in ES cell medium containing 1,400 U ml⁻¹ of leukemia inhibitory factor (LIF) (ESGRO™), Chemicon ESG1106:106 units/ml. [ES medium 500 ml: knock-DMEM (Gibco 10829-018) 425 ml; FCS (ES cell qualified, Gibco 16141-061) 75 ml; MEM non-essential AA solution (Gibco 11140-050) 5 ml; Penicillin-Streptomycin-Glutamin (Gibco 10378-016) 5 ml; 2-mercaptoethanol (Gibco 21985-023) 0.5 ml to final 100 μM on the layer of mouse feeder cells as described in Example 1(b) to grow colonies.]

The colony of HS cells was dissected into several pieces and implanted in one of the two kidney capsules of 26 hybrid mice to induce stemplasm formation. Stemplasms were then harvested by sacrificing the mice in the post-implantation week 1, 3, 6, 9.5, 10.5, 11, 12, and 14. Half of each stemplasm was fixed in formalin for morphological studies, and the other half was frozen in −80° C. for molecular characterization. Stemplasm started to be formed to a visible size around week three. By staggering the harvesting of stemplasms, various tissue types that developed within the stemplasms were studied. All tissue types identified herein were produced within said stemplasms. Stemplasm genotype was verified by PCR-based allelic analysis described in the foregoing paragraphs.

To create embryoid bodies (EB) HS cells on a 60 mm dish were first washed with PBS twice. 1 ml of Trypsin/EDTA solution was then added, and cells were held at a temperature of 37° C. for five minutes. 5 ml of ES medium was then added, and cells were lifted by a cell scraper and spun down at 1000 rpm for five minutes. The cell pellet thus obtained was then resuspended in 5 ml ES medium without LIF, and the cell number was counted. Cells were then seeded onto bacterial culture dishes at 2×10⁶/10 cm dish. Cells were fed in ES medium for 4 days, where medium was changed every two days by transferring cells into 15 ml tubes, waiting about five minutes until the cells settle to the bottom of the tube, then replacing medium. Cells were then aggregated to form EBs and transferred to the original dishes for further differentiation.

### Example 1(e)

### Differentiation of Human HS Cells Within Teratomas, and the Genetic Homozygosity of Such Differentiated Tissue

Thirty-one teratomas were retrieved from the files of the Armed Forces Institute of Pathology, Washington, D.C., and Department of Pathology, New York University. New York, N.Y. (Dr. J. Liu). A variety of different kinds of exclusively differentiated tissue were found in twenty ovarian tumors from female patients. Differentiated tissue was found to be diploid as confirmed by FISH analysis carried out in representative cases using methods known in the art, and alpha-satellite probes to chromosomes 3 and 8. Between 3 and 12 histological areas of undifferentiated and differentiated tissue found in seven ovarian tumors from female patients and four testicular tumors from male patients were identified and selectively microdissected from each case for genetic analysis. In each case, differentiated tissue was found to be genetically homozygous, and undifferentiated tissue was found to be genetically heterozygous.

Microdissection. Unstained 6-micron sections on glass slides were deparaffinized with xylene, rinsed in ethanol from 100% to 80%, briefly stained with hematoxylin and eosin, and rinsed in 10% glycerol in TE buffer. Tissue microdissection was performed under direct light microscopic visualization. From each case, between 6 and 12 areas of different tissue differentiation were separately micro dissected for genetic analysis. In addition, several areas of normal, non-neoplastic tissue were procured.

DNA Extraction. Procured cells were immediately resuspended in 25 μl buffer containing Tris-HCI, pH 8.0; 1.0 mM ethylenediamine tetraacetic acid, pH 8.0; 1% Tween 20, and 0.5 mg/ml proteinase K, and were incubated at 37° C. overnight. The mixture was boiled for 5 minutes to inactivate the proteinase K and 1.5 μl of this solution was used for PCR amplification of the DNA.

Genetic Analysis. In order to reliably identify homozygosity in the limited amounts of DNA that were available after microdissection, multiple different microdissected tissue samples were analyzed with up to 14 distinct highly polymorphic microsatellite markers including DIS1646 and D1S243 (1p), D3S2452 (3p), D5S346 (5q), D7S1822 (7q), Ank-1 (8p), D9S171 (9p), D9S303 (9q), Int-2 and PYGM (11q), IFNA (9p), D17S250 (17q), CYP2D (22q), and AR (Xq). Each PCR sample contained 1.5 μl of template DNA as described above, 10 pmol of each primer, 20 nmol each of dATP, dCTP, DGTP, and DTTP, 15 mM MgCl2, 0.1U Taq DNA polymerase, 0.05 ml [32P]dCTP (6000 Ci/mmol), and 1 μl of 10×buffer in a total volume of 10 μl. PCR was performed with 35 cycles: denaturing at 95° C. for 1 min, annealing for 1 min (annealing temperature between 55° and 60° C. depending on-the marker) and extending at 72° C. for 60 sec. The final extension was continued for 10 minutes. Labeled amplified DNA was mixed with an equal volume of formamide loading dye (95% formamide, 20 mM EDTA, 0.05% bromophenol blue, and 0.05% xylene cyanol).

Samples can be denatured for 5 min at 95%, loaded onto a gel consisting of 6% acrylamide (acrylamide:bisacrylamide 49:1), and electrophoresed at 1800 V for 90 minutes. After electrophoresis, the gels can be transferred to 3 mm Whatman paper and dried. Autoradiography can be performed with Kodak X-OMAT film (Eastman Kodak, Rochester, N.Y.).

Results. Differentiated teratomous tissue showing consistent homozygosity of the same allele included microdissected samples of squamous epithelium, glia, and cartilage (analyzed with markers Ank1 (top) and D1S1646 (bottom)). Normal ovarian tissue was included as control.

In a subset of teratomas, differentiated teratomous tissue found to have discordant homozygous alleles (analyzed with markers Int-2, D9S303, D1S1646, D3S2452, and Ank1) included samples of epidermis, sebaceous gland, respiratory epithelium, and glia. Normal ovarian tissue was included as a control. In such tumors, it is believed that allelic heterozygosity results from the initiation of tumorigenesis before meiosis I in germ cells. After teratogenic tumor cell initiation, random, independent events then lead to progenitor cells with a postmeiotic genotype.

A series of ovarian teratomas and testicular germ cell tumors containing both differentiated and undifferentiated tissue were also analyzed. In each tumor, both undifferentiated and differentiated tissue elements were procured. Homozygous and heterozygous components were detected using markers D3S2452, D3S303, CYP2D, and D17S250. Normal ovarian and testicular tissues were included as controls. Heterozygous alleles were detected in undifferentiated tissue elements including immature squamous epithelium, neural tissue (sometimes from separate areas of neural tissue within the same tumor), cartilage, glandular structures, and mesenchyme. Differentiated tissue elements isolated from the same tumors by microdissection were found to be homozygous for the same markers. Mature elements tested included: sebaceous gland tissue, hair follicle, and mature squamous epithelium (sometimes from separate areas of squamous epithelium within the same tumor). In some tumors, differentiated elements showed opposite homozygous alleles, indicating recombination or suggesting that various elements arose separately from distinct postmeiotic cells.

### Example 1(f)

### Derivation of Progenitor Cells from Human HS Cells Primary Differentiation

HS cells grown on 60 mm dish (Falcon, #353802) with primary embryonic fibroblast layer and/or 0.1 % gelatin coated dishes are trypsinized with 1.5 ml Trypsin/EDTA (Invitrogen, #25300-050) and transferred to 1.5 ml ES-LIF medium in a 15 ml conical tube. Cells are then spun down at 1200 rpm, and the supernatant is removed. The cell pellet is resuspended into single cell suspension in 2 ml ES-LIF medium, and cultured as suspension cells in suspension culture-35*10 mm-dishes (NalgeNunc, #171099) at a density of 1-3×10⁶ cells to allow stem cells to form rounded spherical clusters, known as embryoid bodies (EBs) for 4-6 days. Forming EBs are washed every two days by transferring the EBs to 15 ml conical tubes, and then allowed to settle to the bottom. The supernatant is removed and new ES-LIF is added. EBs are then transferred back into suspension culture dish. HS cells grown as embryoid bodies are comprised of all the germ cell layers, ectodermal, endodermal, and mesodermal.

Ectodermal Progenitors. After 4-6 days, EBs are trypsinized in 1 ml of Trypsin/EDTA, washed in 4 ml ES-LIF medium, and resuspended into single cell suspension in DMEM/Knockout medium (Invitrogen, #10829-018) supplemented with 10% Serum Replacement (Invitrogen, #10828), and G5 (Invitrogen, #17503), N2 (Invitrogen, #I7502-048) or beta NGF (10 ng/ml) (R&D Systems, #256-GF). These cells are cultured at 3-5×10⁵/3 ml in fibronectin-coated 35 mm dishes (50 ug/ml)(Sigma, #F-0895) for 10 days, with media changes every two-three days.

Alternatively, the EBs are cultured in 0.1% gelatin-coated dish in ES-LIF medium for 1-2 days, and then the medium is changed to serum-free medium supplemented with Insulin (5 ug/ml), Selenium chloride (0.015 nM), Transferrin (50 ug/ml), and fibronectin (5 ug/ml)(Sigma) for 6 days. The cells are trypsinized, and single cell suspensions are cultured in N2 medium (serum free-DMEM/F12 supplemented with N2 (Invitrogen, #I7502-048), B27 (Invitrogen, #I7504-44), and bFGF (10 ng/mL) (Invitrogen, #13256-029)). Cells are then counted and seeded at a density of 2-5×10⁴ cells/well/400 uL N2 medium in 24-well plates pre-coated with poly-L-ornithine (15 ug/ml)(Sigma, #P36550), and expanded for six days.

These progenitors are further differentiated into different neuronal cell types by adding G5, RA, FGF, NGF, GNDF, or BNDF. They are also maintained in their presence conditioned media for cell expansion.

Mesodermal Progenitors. For mesodermal progenitors, the single cell suspension in DMEM/Knockout medium supplemented with 10% Serum Replacement and beta-NGF as described above are cultured for 10 days with media change every two/three days. After this period, the cells are further cultured in Activin A supplemented (20 ng/ml) (Sigma, #A4941) conditioned medium for another 10 days for heart progenitor cells. Alternatively, for kidney and Mullerian duct progenitor cells the cells are further cultured in Activin A supplemented (20 ng/ml) (Sigma, #A4941) conditioned medium for 4-6 days after which 2 ng/ml of TGF-beta (R&D Systems, #) is added to the medium, and the cells are cultured for another 4-6 days.

Endodermal Progenitors. For endodermal progenitors, the single cell suspension in DMEM/Knockout medium supplemented with 10% Serum Replacement, along with G5 or beta-NGF on laminin-coated (10 ug/ml)(Sigma, #L2020), or Collagen I-coated (10 ug/ml)(Sigma, #C-7661) is cultured for 10 days. HGF (20 ng/ml) and/or TGF-alpha (2 ng/ml) are added to the medium to replace G5 or beta-NGF, and the cells are cultured for another 6-8 days.

Alternatively, EBs are plated onto Collagen I-coated dishes and cultured in ES-LIF medium for 4 days. FGF (20 ng/ml) is added and the cells are cultured for another 3 days. After this period, HGF (20 ng/ml) and/or TGF-alpha (2 ng/ml) are added and cultured for another 6 days.

EBs are also transferred to laminin-coated adherent dishes (10 ng/ml) (Sigma, #L2020) or 0.1% gelatin coated 35*10 mm adherent dish, and cultured 1-2 days in ES-LIF medium. The medium is removed and serum-free DMEM/F12 (Invitrogen, #11330-0321) medium supplemented with Insulin (5 ug/ml)(Invitrogen, #11882), Selenium chloride (0.015 nM)(Sigma, #S5261), Transferrin (50 ug/ml) (Sigma, #T-2036), and Fibronectin (5 ug/ml) (Sigma). This medium is designated as ITSFn medium. Cells are fed for 6 days in ITSFn medium, where medium is changed every two days.

### Example 1(g)

### Development and Isolation of Homozygous Progenitor Cells from Transplanted HS Cells

To obtain homozygous progenitor cells, pluripotent HS cells derived from methods disclosed in the foregoing description and examples are transplanted into immuno-compromised mice under kidney capsules and are allowed to grow in vivo for 4 to 6 weeks. The cell mass obtained is then minced into single cells and cultured on feeder cells for further propagation and development into cell lines.

To assess the lineage commitment (the types of progenitor cells ), gene expression assays, such as RT-PCR, northern blot, immunohistochemistry, and so forth, are performed for known lineage-specific markers, for example, NF-H, keratin, D-beta-H for the ectoderm, enolase, CMP, rennin, kallikerein, WT1, delta-globin, beta-globin for the mesoderm, and albumin, alpha-1-AT, amylase, PDX-1, insulin, alpha-FP for the endoderm progenitor lineages.

### Example 1(g)(i)

### Maintaining Human HS Cell Lines and Development and Isolation of Homozygous Progenitor Cells from in vivo Growth of Established Human HS Cell Lines

This work can be used for (1) maintaining human HS cells growth in vivo instead of in vitro, so that their genome stability and pluripotency can be better preserved, and (2) deriving progenitor cell lines in vivo from established HS lines.

HS cells, derived from methods disclosed in foregoing description and examples, are transplanted into kidney capsules or subcutaneously into the hind-legs of immuno-compromised mice (a.k.a. nude mice) for in vivo growth of stemplasms. The cell masses developed after 4 to 6 weeks growth are harvested and then minced into single cells and culture on mitotically inhibited mouse embryonic fibroblasts for colony formation and isolation. The isolated colonies are further propagated and maintained in vitro. To characterize each colony for its status of pluripotency, gene expression profile analysis such as RT-PCR, northern blot, immunocytochemistry (on known lineage specific markers), and telomerase activity analysis are used for assessing the stem cell characteristics and lineage commitment. Examples of gene expression analysis for lineage commitment are: SSEAs, Oct4, alkaline phosphatase, telomerase for undifferentiated stem cells; NF-H, keratin, D-beta-H for ectodermal progenitor cells; enolase, CMP, rennin, kallikerein, WT1, delta-globin, beta-globin for mesodermal progenitor cells; and albumin, alpha-1-AT, amylase, PDX-1, insulin, alpha-FP for endodermal progenitor cells. Alternatively, the cells from isolated colonies are transplanted back into said immuno-compromised mice for extensive growth and differentiation. Histological examinations of the resultant differentiated tissue masses are used to extrapolate stem cell characteristics and lineage commitment of the colonies.

### Example 1(g)(ii)

### Maintaining Mouse HS Cell Lines and Development and Isolation of Homozygous Progenitor Cells from in vivo Growth of Established Mouse HS Cell Lines

This work can be used for (1) maintaining mouse HS cells growth in vivo instead of in vitro, so that their genome stability and pluripotency can be better preserved, and (2) deriving progenitor cell lines in vivo from established HS lines.

HS cells, derived from methods disclosed in foregoing description and examples, are transplanted into kidney capsules or subcutaneously into the hind-legs of immuno-compromised mice (a.k.a. nude mice) or syngenic mice for in vivo growth of stemplasms. The cell masses developed after 4 to 6 weeks growth are harvested, then minced into single cells, and cultured on mitotically inhibited mouse embryonic fibroblasts for colony formation and isolation. The isolated colonies are further propagated and maintained in vitro. To characterize the each colony for its status of pluripotency, gene expression profile analysis such as RT-PCR, northern blot, immunocytochemistry (on known lineage specific markers), and telomerase activity analysis are used for assessing stem cell charactieristics and lineage commitment. Examples of gene expression analysis for lineage commitment are: SSEAs, Oct4, alkaline phosphatase, telomerase for undifferentiated stem cells; NF-H, keratin, D-beta-H for ectodermal progenitor cells; enolase, CMP, rennin, kallikerein, WT1, delta-globin, beta-globin for mesodermal progenitor cells; and albumin, alpha-1-AT, amylase, PDX-1, insulin, alpha-FP for endodermal progenitor cells. Alternatively, the cells from isolated colonies are transplanted back into immuno-compromised mice for extensive growth and differentiation. Histological examinations of he resultant differentiated tissue masses are used to extrapolate stem cell characteristics and lineage commitment of the colonies.

### Example 1(g)(iii)

### Development and Isolation of Human Homozygous Stem Cell Lines, and Progenitor Cell Lines from in vivo Growth of Derivatives of Mitotically Activated Human Post-Meoisis I Oocytes

Alternative to the foregoing description of in vitro human HS cell lines formation method (examples l(b), (c)), derivatives of mitotically activated human post-meiosis I oocytes, including one-cell, two-cell, four-cell, eight-cell, sixteen-cell, morulae, pre-hatching blastocyst, and post-hatching blastocyst, post-immunosurgery blastocyst, and inner cell mass growth, are transplanted into immuno-compromised mice (a.k.a. nude mice) for in vivo growth in the uterus, kidney capsules, or subcutaneously into the dermis of hind-legs.

The cell masses developed after 4 to 6 weeks growth are harvested and then minced into single cells and culture on mitotically inhibited mouse embryonic fibroblasts for colony formation and isolation. The isolated colonies are further propagated and maintained in vitro. To characterize each colony for its status of pluripotency, gene expression profile analysis such as RT-PCR, northern blot, immunocytochemistry (on known lineage specific markers), and telomerase activity analysis are used for assessing the stem cell characteristics and lineage commitment. Examples of gene expression analysis for lineage commitment are: SSEAs, Oct4, alkaline phosphatase, telomerase for undifferentiated stem cells; NF-H, keratin, D-beta-H for ectodermal progenitor cells; enolase, CMP, rennin, kallikerein, WT1, delta-globin, beta-globin for mesodermal progenitor cells; and albumin, alpha-1-AT, amylase, PDX-1, insulin, alpha-FP for endodermal progenitor cells. Alternatively, the cells from isolated colonies are transplanted back into nude mice for extensive growth and differentiation. Histological examinations of he resultant differentiated tissue masses are used to extrapolate the stemness and lineage commitment of the colonies.

### Example 1(g)(iv)

### Development and Isolation of Mouse Homozygous Stem Cell Lines, and Progenitor Cell Lines from in vivo Growth of derivatives of Mitotically Activated Mouse Post-Meoisis I Oocytes

Another alternative to the foregoing description of in vitro mouse HS cell line formation methods (example l(a)), derivatives of mitotically activated human post-meiosis I oocytes, including one-cell, two-cell, four-cell, eight-cell, sixteen-cell, morulae, pre-hatching blastocyst, and post-hatching blastocyst, post-immunosurgery blastocyst, and inner cell mass growth, are transplanted into immuno-compromised (nude) or syngenic mice for in vivo growth in the uterus, kidney capsules, or subcutaneously into the dermis of hind-legs.

The cell masses developed after 4 to 6 weeks growth are harvested and then minced into single cells and culture on mitotically inhibited mouse embryonic fibroblasts for colony formation and isolation. The isolated colonies are further propagated and maintained in vitro. To characterize the each colony for its status of pluripotency, gene expression profile analysis such as RT-PCR, northern blot, immunocytochemistry (on known lineage specific markers), and telomerase activity analysis are used for assessing the stem cell characteristics and lineage commitment. Examples of gene expression analysis for lineage commitment are: SSEAs, Oct4, alkaline phosphatase, telomerase for undifferentiated stem cells; NF-H, keratin, D-beta-H for ectodermal progenitor cells; enolase, CMP, rennin, kallikerein, WT1, delta-globin, beta-globin for mesodermal progenitor cells; and albumin, alpha-1-AT, amylase, PDX-1, insulin, alpha-FP for endodermal progenitor cells. Alternatively, the cells from isolated colonies are transplanted back into nude mice for extensive growth and differentiation. Histological examinations of the resultant differentiated tissue masses are used to extrapolate the stem cell characteristics and lineage commitment of the colonies.

### Example 1(g)(v)

### Isolation of Human Homozygous Stem Cell Lines, and Progenitor Cell Lines from Human Teratomas

Surgically removed human teratomas are minced into single cells and culture on mitotically inhibited mouse embryonic fibroblasts for colony formation and isolation. The isolated colonies are further propagated and maintain in vitro.

Haplotype analysis described in example 1(e) is used to characterize the homozygosity of the isolated colonies. Colonies with homozygosity in the genetic markers analyzed are further propagated in vitro or in vivo (example 1(g)(i)).

To characterize the each colony for its status of pluripotency, gene expression profile analysis such as RT-PCR, northern blot, immunocytochemistry (on known lineage specific markers), and telomerase activity analysis are used for assessing the stem cell charateristics and lineage commitment. Examples of gene expression analysis for lineage commitment are: SSEAs, Oct4, alkaline phosphatase, telomerase for undifferentiated stem cells; NF-H, keratin, D-beta-H for ectodermal progenitor cells; enolase, CMP, rennin, kallikerein, WT1, delta-globin, beta-globin for mesodermal progenitor cells; and albumin, alpha-1-AT, amylase, PDX-1, insulin, alpha-FP for endodermal progenitor cells. Alternatively, the cells from isolated colonies are transplanted back into nude mice for extensive growth and differentiation. Histological examinations of he resultant differentiated tissue masses are used to extrapolate the stem cell characteristics and lineage commitment of the colonies.

### Example 1(h)

### Development and Isolation of Homozygous Stem Cells, and Progenitor Cells from Transplanted HS Cells

To obtain homozygous stem cells and multi-potent progenitor cells, pluripotent HS cells derived from methods disclosed in the foregoing description and examples are injected into the hind limbs of immuno-compromised mice using matrigel tissue medium, and are allowed to grow in vivo for 4 to 6 weeks. The cell mass obtained is then dissected, minced into single cells, and plated in culture on matrigel for further propagation and development into cell lines. Colonies of both homozygous stem cells, and homozygous progenitor cells were observed.

To assess homozygosity, genotyping was performed, and to assess lineage commitment (the types of progenitor cells), gene expression assays, such as RT-PCR, northern blot, immunohistochemistry, and so forth, are performed for known lineage-specific markers, for example, NF-H, keratin, D-beta-H for the ectoderm, enolase, CMP, rennin, kallikerein, WT1, delta-globin, beta-globin for the mesoderm, and albumin, alpha-1-AT, amylase, PDX-1, insulin, alpha-FP for endoderm progenitor lineages.

### Example 2

### Differentiation of Mouse HS Cells into Cells from the Mesodermal Embryonic Layer

### Example 2(a)

### Differentiation into Hematopoietic Cells

Mouse HS cells were cultured in ES medium (DMEM Gibco 1195-065; FBS Gibco 16141-079, 100 μM Non-Essential amino acid Gibco 11140-050; 50 units/ml Penicillin-Streptomycin Gibco 15070-063; 100 μM β-Mercaptoethanol Gibco 21985-023)with LIF (1000 IU/ml) for 3-5 days. The cells were then trypsinized with Trypsin/EDTA (Gibco 25300-054, 1 ml/60 mm dish) for 5 minutes at 37° C. and 5 ml of ES medium was added. The mouse stem cells were lifted from the dish by cell scraper and the cell suspension was spun at 1000 rpm for 5 minutes. The cell pellet obtained was resuspended in ES medium without LIF and with 4.5×10⁻⁴ M MTG (monothioglyceral Sigma M6145) at the cell concentration of 2X10⁶/10 cm dish for 4 days at 37° C. and 5% CO₂. Mouse HS cells were then aggregating in suspension to form embryoid bodies (EBs).

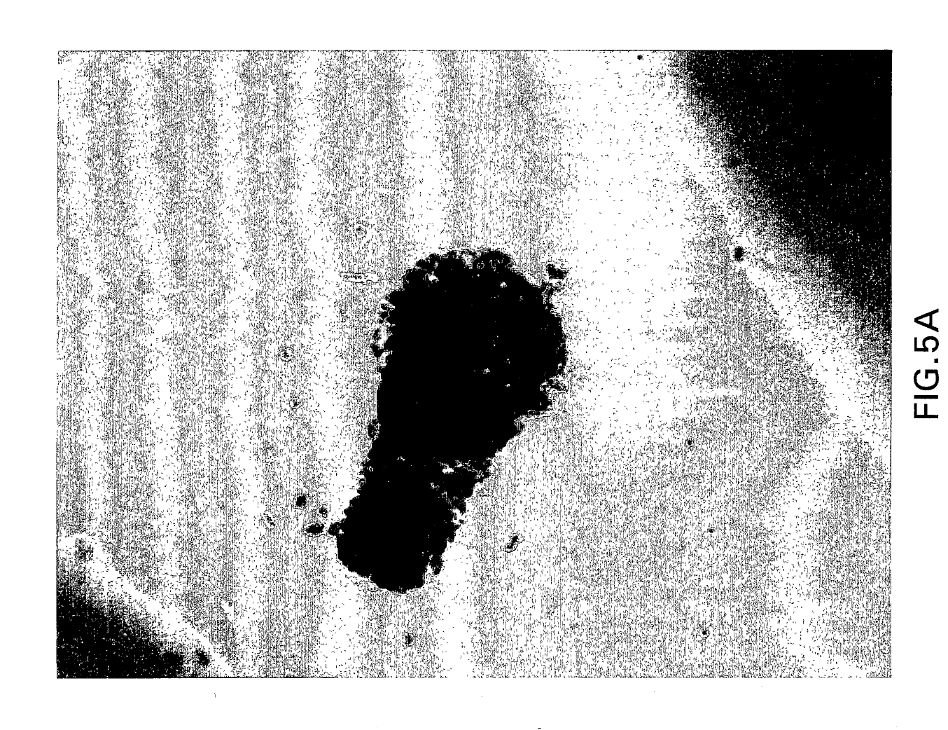

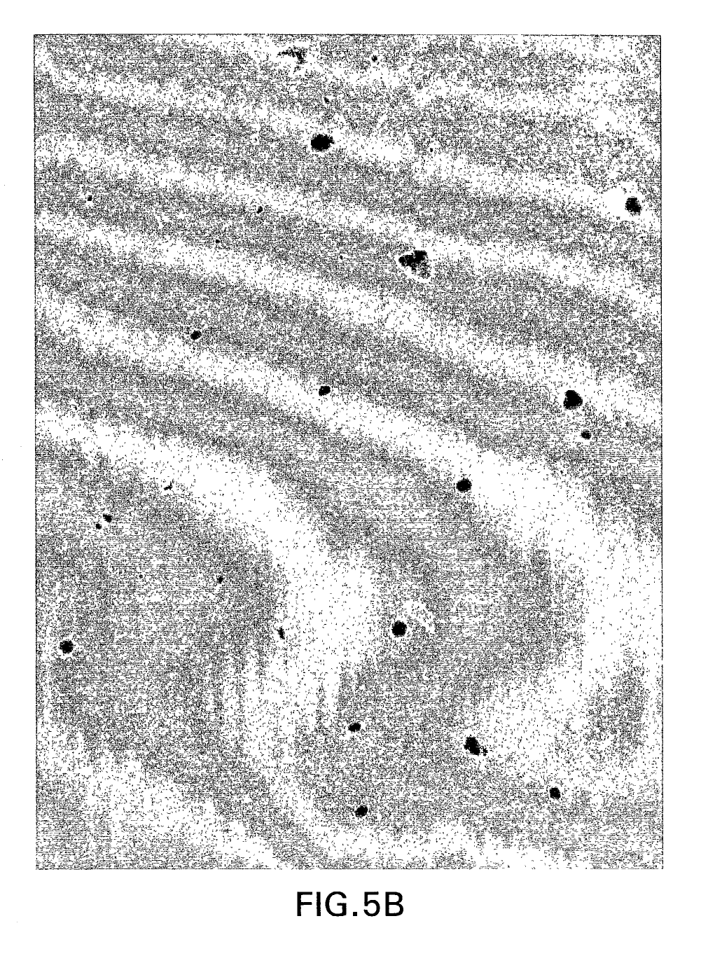

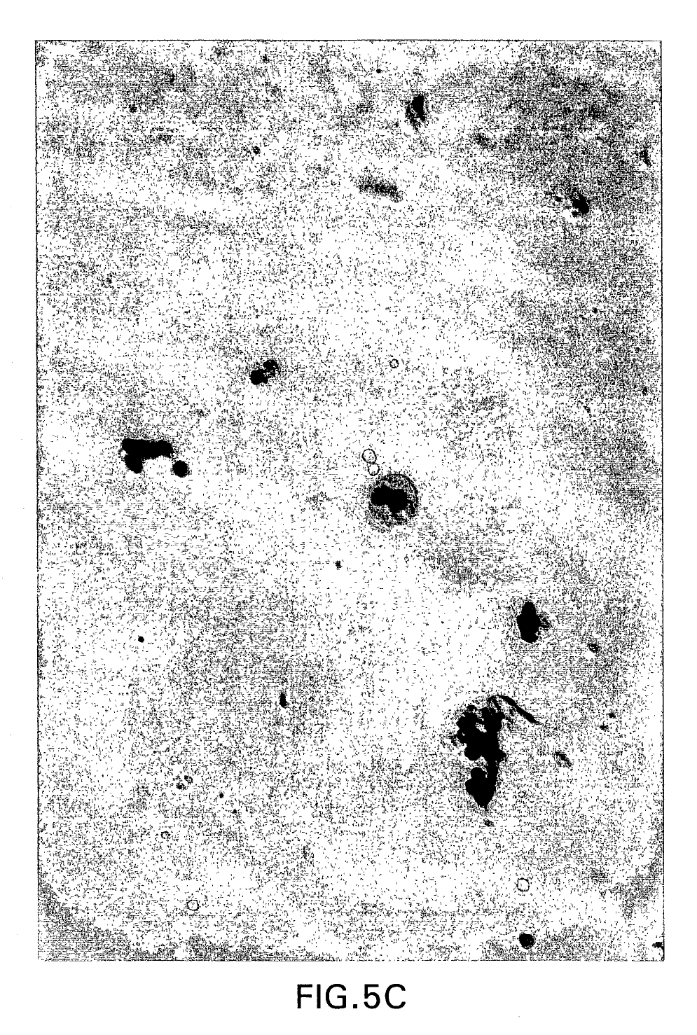

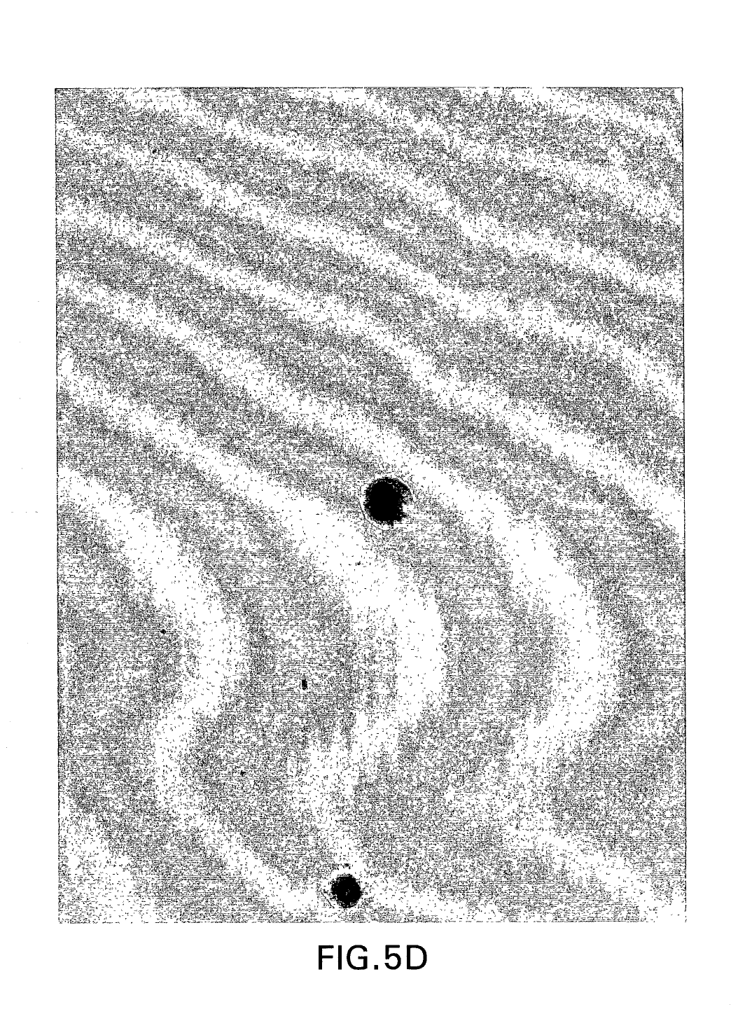

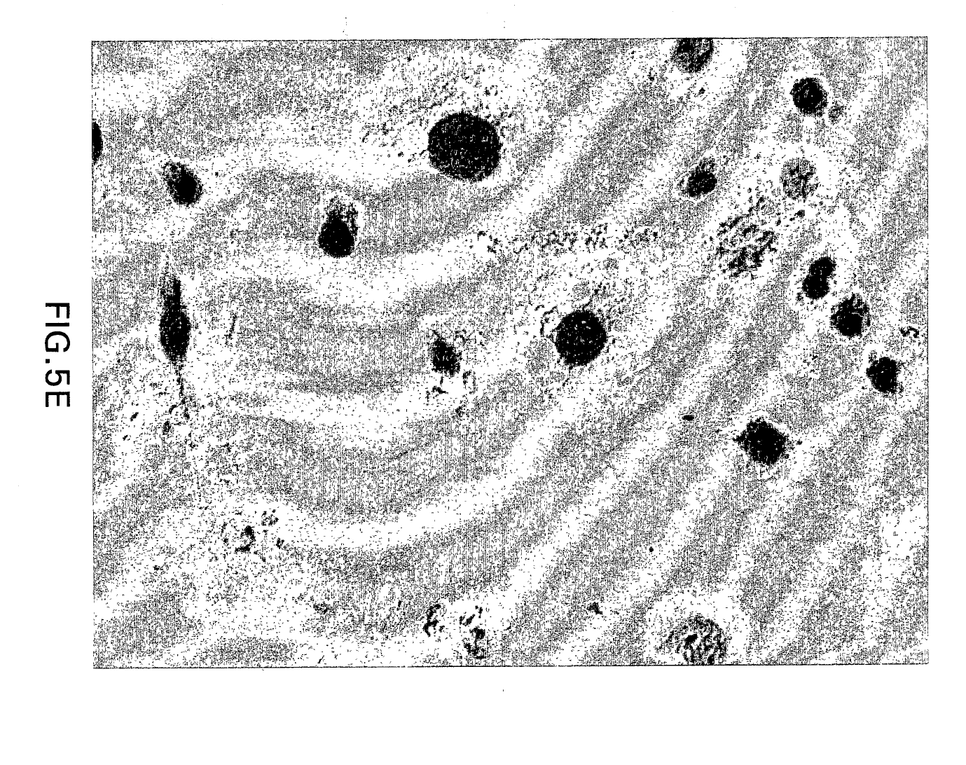

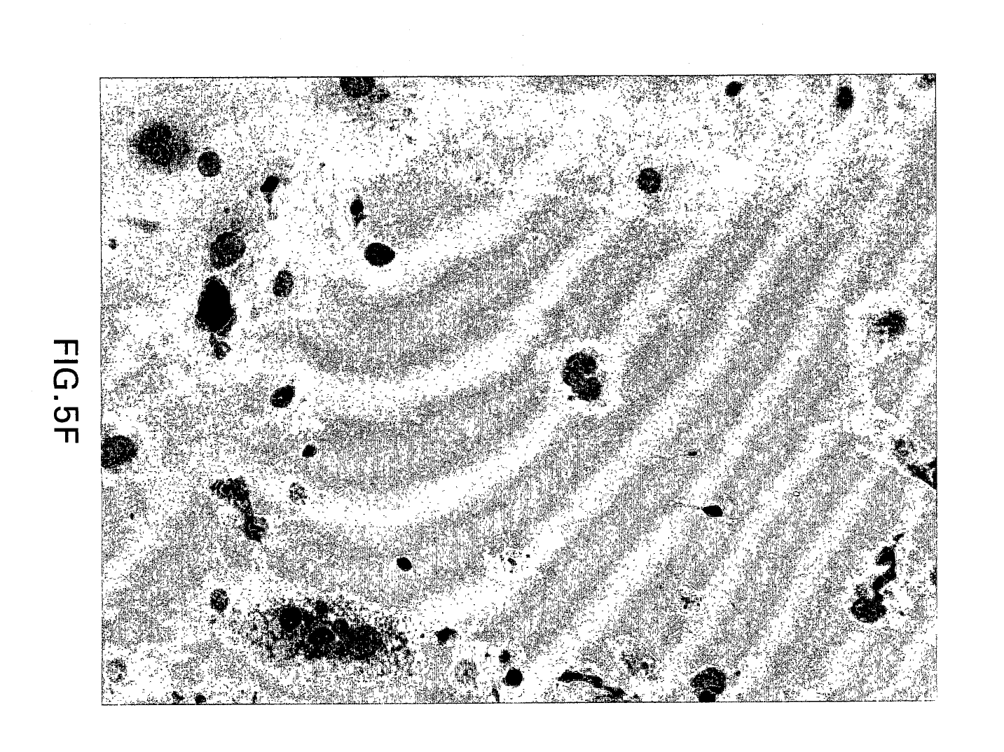

30-40 EBs formed were transferred to a 35 mm dish with 3 ml methylcellulose based hemopoietic cell differentiation medium M3434 (Stemcell 03434), which contains fetal bovine serum, bovine serum albumin, bovine pancreatic insulin, human transferrin (iron-saturated), β-mercaptoehtanol, L-glutamine, rm IL-3, rh IL-6, rm SCF and rh-erythropoietin and incubated at 37° C. and 5% CO₂. After 10 days incubation, several colonies and different type of cells were picked by pipette tips and resuspended in 500 μl IMDM medium (Sigma 13390). The mixture was then transferred into a 4-well chamber-slide and the colonies and cells were attached to the slide by incubating the chamber slide at 37° C. for at least 3 hours. After the cells attached to the slide, the IMDM medium was discarded and the cells were fixed in methanol for 7 minutes. The slide was air dried after methanol fixation, stained with 1:20 diluted Giemsa stain (Sigma GS-500) for 30 minutes at room temperature, and then rinsed 3 times with water. Observation of colonies and different types of hematopoietic cells started after 10 days in culture. See FIG. 5A for CFU (colony formation unit), FIG. 5B for erythrocytes, FIG. 5C for monocytes, and FIG. 5D for lymphocytes obtained using the protocol described above.

EBs grown in M3434 for 10-15 days were also transferred to 35 mm dish with IMDM, 10% FBS and either IL-3 (Stemcell 02733) alone or a combination of IL-3 and GM-CSF (Stemcell 02732). The cells were fixed and stained as described above and observation of the cell differentiation from EBs started within 5 days in liquid IMDM with cytokines. The cells differentiated from IMDM with IL-3 contained granules but no monocytes, and the cells from IMDM with IL-3 and GM-CSF contained granules and some monocytes (See FIGS. 5E & 5F).

### Example 2(b)

### Differentiation into Spontaneously Contracting Muscle Cells

Mouse HS cells were cultured in ES medium (DMEM Gibco 1195-065; FBS Gibco 16141-079, 100 μM Non-Essential amino acid Gibco 11140-050; 50 units/ml Penicillin-Streptomycin Gibco 15070-063; 100 μM β-Mercaptoethanol Gibco 21985-023) with LIF (1000 IU/ml) for 3-5 days. The cells were then trypsinized with Trypsin/EDTA (Gibco 25300-054, 1 ml/60 mm dish) for 5 minutes at 37° C. and 5 ml of ES medium was added. The mouse stem cells were lifted from the dish by cell scraper and the cell suspension was spun at 1000 rpm for 5 minutes. The cell pellet obtained was resuspended in ES medium without LIF at the cell concentration of 2×10⁶/10 cm dish for 4 days at 37° C. and 5% CO₂. Mouse HS cells were then aggregated in suspension to form embryoid bodies (EBs).

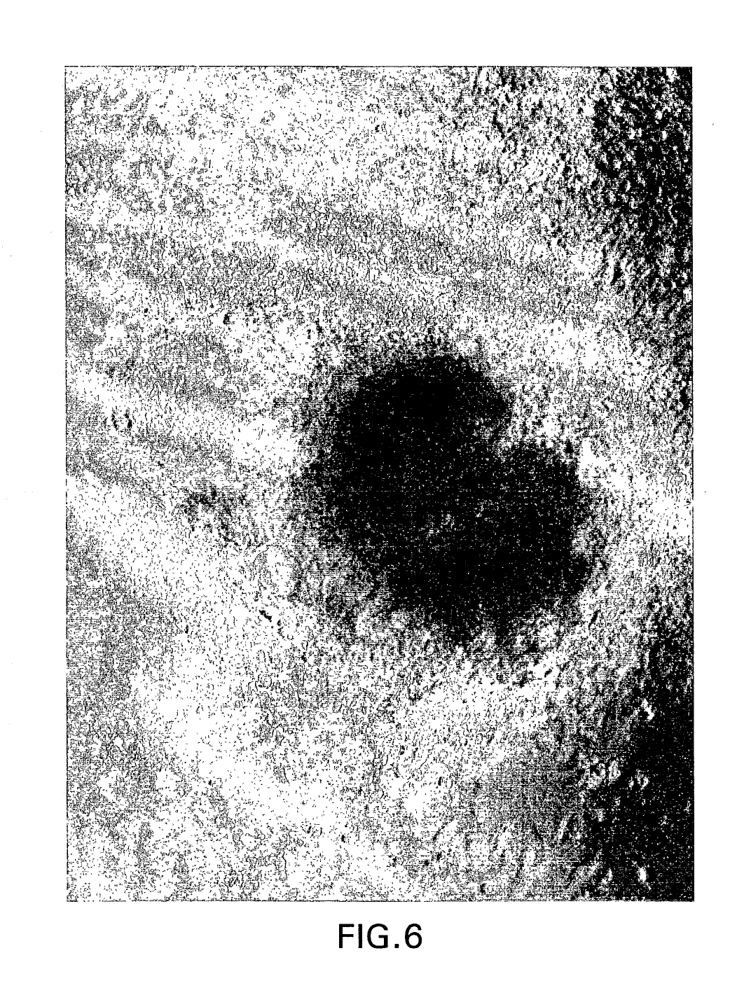

10-15 EBs formed were transferred to 24-well plate coated with gelatin. (1 ml of 0.1% gelatin was added into each well and allowed to sit in the hood for more than two hours. The gelatin is then removed and the plate is allowed to dry for 30 minutes.), and incubated in LIF-free ES medium which was changed every two days. Around the fourth day spontaneously contracting cells or beating EBs appeared and continued to beat up to 3-4 days. (See FIG. 6).

In addition, the beating EBs were induced by incubating EBs obtained as described above in semisolid medium (with 1% methylcellulose) called M3434 (Stemcell 03434), which contains fetal bovine serum, bovine serum albumin, bovine pancreatic insulin, human transferrin (iron-saturated), β-mercaptoehtanol, L-glutamine, rm IL-3, rh IL-6, rm SCF and rh-erythropoietin at 37° C. and 5% CO₂ The beating EBs were observed after 3-4 days incubation.

The beating EBs were also formed in serum-free ITSFn medium, which contains DMEM/F12 (Gibco 11320-033) containing insulin (5 μg/ml, Sigma 11882), Selenium chloride (30 nM, Sigma S5261) and Fibronectin (5 μg/ml, Sigma F 1141), after 4 days incubation of the above described EBs at 37° C. and 5% CO₂.

### Example 3

### Differentiation of Mouse HS Cells into Cells from the Endodermal Embryonic Layer

### Example 3(a)

### Differentiation into Pancreatic Cells

HS cells grown on 60 mm dish (Falcon, #353802) with primary embryonic fibroblast layer and/or 0.1% gelatin coated dishes were trypsinized with 1.5 ml Trypsin/EDTA (Invitrogen, #25300-050), and transferred to 1.5 ml ES-LIF medium in 15 ml conical tube. Cells were then spun down at 1000 rpm, and the supernatant was removed.

The cell pellet obtained was resuspended into single cell suspension in 2 ml ES-LIF medium, and cultured as suspension cells in suspension culture-35*10 mm-dishes (NalgeNunc, #171099) to allow stem cells to form rounded spherical clusters, known as embryoid bodies (EBs) for 4-6 days. Forming EBs were washed every two days by transferring the EBs to 15 ml conical tubes. The EBs were allowed to settle to the bottom, and the supernatant was removed. New ES-LIF was then added, and EBs were transferred back into suspension culture dish. The HS cells grown as embryoid bodies comprised of all the germ cell layers, ectodermal, endodermal, and mesodermal. Selection of Pancreatic precursor cells. After 4-6 days in culture as embryoid bodies, cells were transferred to 15 ml conical tubes, and allowed to settle. Half of the medium was removed and 1.5 ml of fresh ES-LIF medium was added to tube followed by transferring the entire content of the tube to a 0.1% gelatin coated 35*10 mm adherent dish for overnight culture. ES-LIF medium was removed the next day and serum-free DMEM/F12 (Invitrogen, #11330-0321) medium supplemented with Insulin (5 ug/ml) (Sigma, #I-1882), Selenium chloride (0.015 nM)(Sigma, #S5261), Transferrin (50 ug/ml) (Sigma, #T-2036), and Fibronectin (5 ug/ml)(Sigma, #F-0895). This medium is designated as ITSFn medium. Cells were fed for 6 days in ITSFn medium, where medium was changed every two days.

EBs were also cultured on laminin-coated adherent dishes (10 ng/ml) (Sigma, #L-2020) in ES-LIF medium for two to four days to allow the endodermal cells to migrate out of the embryoid bodies, expanded in 8.7 mM glucose DMEM/F12 serum-free medium that were supplemented with N2 (Invitrogen, #I7502-048), B27 (Invitrogen cat#I7504-44), and bFGF (10 ng/mL)(Invitrogen, #13256-029) for 4 days. After the endoderm expansion, the medium was changed to ITSFn medium, and grown for 6 days with medium change every two days to select for pancreatic precursor cells.

Alternatively, after 4-6 days EBs were trypsinized in 1 ml of Trypsin/EDTA, washed in ES-LIF medium, and resuspended into single cell suspension in DMEM/Knockout medium (Invitrogen, #10829-018) supplemented with 10% Serum Replacement (Invitrogen, #10828), and G5 (Invitrogen, #17503), or beta NGF (R&D Systems, #256-GF). These cells were culture at 3-5×10⁵/3 ml in fibronectin-coated 35 mm dishes (10 ug/ml) for 4-6 days with media changes every two days. After 4-6 days, ITSFn medium was added to replace the DMEM/Knockout medium, and cells were cultured for another 6 days for selection of pancreatic precursor cells.

The pancreatic precursor cells are positive for the early markers, Nestin, neurogenin 3, and tyrosine hydroxylase.

Expansion of Pancreatic Precursor Cells by bFGF. Cells maintained in ITSFn medium were washed twice with PBS, after removing the medium. 1 mL of Trypsin/EDTA was added, and cells were incubated at 37° C. for 5 minutes to cause dissociation. The adhered cells were further dissociated by using cell scraper. 3 ml of ES-LIF medium was then added to dish, and its entire content was transferred to 15 ml conical tube. Remaining EBs from the pancreatic precursor selection were allowed to settle for about 2-5 minutes, and the supernatant was transferred to a new 15 ml conical tube and spun down at 1200 rpm. The supernatant was discarded and the cell pellet was resuspended into serum-free DMEM/F12 medium, at 5.8 mM glucose or lower, supplemented with N2 (Invitrogen, #I7502-048), B27 (Invitrogen, #I7504-44), and bFGF (10 ng/mL)(Invitrogen, #13256-029). Such medium was designated as N2 medium.

Cells were counted and seeded at a density of 2-5×10⁵ cells/well/400 uL N2 medium in 24 well plated pre-coated with poly-L-ornithine (15 ug/ml)(Sigma, #P36550), and expanded for six to eight days. Alternatively, cells were seeded at a density of 2-5×10⁴ cells/well/400 ul N2 medium and expanded for eight to ten days.

The precoating protocol was as follows: 400 ul of 15 ug/ml of poly-L-ornithine was added to each well of 24-well plates and let sit at room temperature overnight; plates were then washed with PBS twice, fresh PBS was added and plates were incubated at 37° C. for 30 minutes; plates were washed with PBS, and 400 ul of Fibronectin (10 ug/ml) was added followed by incubating the plates at room temperature for at least two hours before use.

Differentiation of Pancreatic Precursors into Insulin-Secreting Beta-Islets Cells.

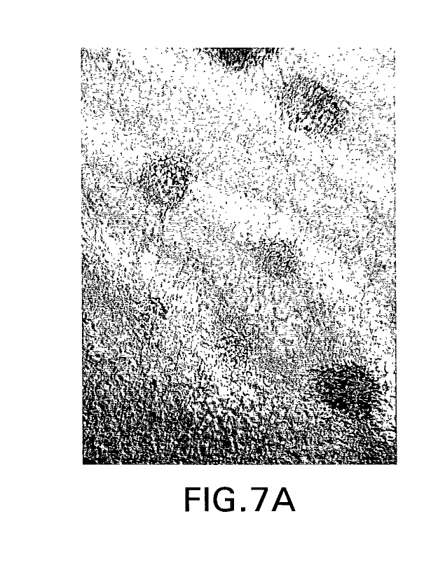

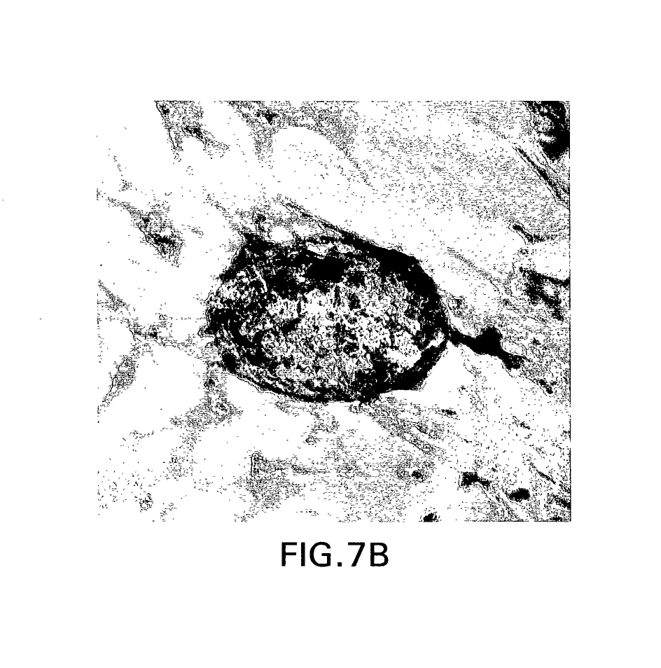

Pancreatic precursors were driven to differentiate into Insulin-secreting beta islets cells by withdrawing bFGF from N2 medium, and in the presence of 100 ng/ml EGF (Invitrogen, #53003-018), 20 ng/ml HGF (Sigma, #H1404), and 20 ng/ml Activin A (Sigma,#A4941) or 20 ng/ml VEGF (R&D Systems, #298-VS). Cells were allowed to differentiate for six days with medium changes every two days. Upon differentiation, the epithelial pancreatic cells gave rise to small rounded cells, which underwent rapid proliferation to form organized cell clusters, appeared as smooth spheroids, see FIG. 7A.

Detection of Insulin-secreting Beta Islets. For detection of insulin production and secretion, differentiation medium was removed and replaced with high glucose DMEM/F12 supplemented 10 mM Nicotinamide, 0.015 nM Selenium chloride, 50 ug/ml Transferrin, 0.1 mM putrescine (Sigma, #P5780), and 20 nM progesterone (Sigma, #P8783). These cells are then cultured for 3 hours at 37° C. After three hours, medium in each well was collected and stored at −70° C. for insulin release assay, and cells in each well were fixed in 4% paraformaldehyde (EMS, #15712) for 30 minutes at room temperature for immunocytochemistry, or RNA from each well is collected by RNAzol (Tel-Test, Inc., Friendswood, Tex., #CS-105) for RT-PCR analysis of gene expression. In some experiments, the insulin content of the pancreatic-spheroid clusters is measured, instead of immunocytochemistry or RT-PCR, by overnight acid-ethanol extraction at 4° C. Cell-free extracts are collected, neutralized with 0.4M Tris-base, and stored at −70° C. for insulin content assays.

Immunocytochemistry. After 30 minutes fixation in 4% paraformaldehyde at room temperature, cells were washed three times in PBS (Biofluids, #P312-500). These cells were further fixed and permeablized in 100% methanol (Fischer Scientific, #HC400-1 gal) for 5 min at room temperature. Cells were then washed three more times in PBS and blocked with block solution (DAKO Envision double stain system, #K1395) for five minutes. Excess block buffer was tapped off, primary antibody was applied, and cells were then incubated for two hours at room temperature. For Insulin, the primary antibody used was polyclonal guinea pig anti-insulin (DAKO, #A0564) at 1:50 dilution. The dilution was made in Medium B (Caltag, #GAS002).

Cells were then rinsed three times with PBS, HRP-conjugated secondary antibody was applied, Bottle 2, (DAKO, #K1395) followed by incubating cells for 30 minutes. Cells were rinsed three times with PBS, excess liquid was absorbed, and liquid DAB+chromogen substrate, Bottle 3, was added for five to ten minutes. Finally, cells were rinsed again with PBS and examined under a microscope, see FIG. 7B.

Double staining for Glucagon, 1:300 (DAKO, #A0565), was done following the DAKO Envision double staining protocol, see FIG. 7B). Pax6, 1:300 (Covance, #PRB-278P), and antibodies to mark other cell types are also used. Alternatively, all immunostaining is performed using fluorochrome-conjugated secondary antibodies (Sigma, Molecular Probes, or Jackson Labs), and visualized under Leica inverted-fluorescence microscope.

Insulin Release and Cell-content Detection Assay: To measure insulin protein secretion or insulin content of pancreatic spheroid-clusters, the medium or ethanol-extract collected from each well is applied to the enzyme-linked immunosorbent assay, ELISA, (Crystalchem, Chicago, Ill.).

### Example 3(b)

### Differentiation of HS Cells into Hepatic Cells

The following protocol for hepatic cell differentiation of HS cells is based on Hamazaki et al, “Hepatic Maturation in Differentiating Embryonic Stem Cells in vitro”, FEBS Lett. 497(1):15-9 (2001).

Homozygous stem cells are plated on mitomycin treated mouse embryonic fibroblasts (STO cells) on tissue cultures dishes (FALCON 35-3802, 60×15 mm style, polystyrene, nonpyrogenic, Becton Dickinson Labware) in stem cell medium containing 20% fetal bovine serum (Life technologies) in DMEM medium supplemented with nonessential amino acid, pen-strep (Life Technologies), beta-mercaptoethanol (Sigma), and LIF (Chemicon). Cells are cultured at 37° C., 5% C02 overnight. HS cells are then trypsinized with Trypsin-EDTA (0.05%-0.5%) (Life Technologies) and cultured in suspension dishes (Suspension Dish with Lid and Vent, Nalge Nunc International, 171099, 35×10 mm) for embryoid body formation in the same medium without LIF for 5 days. The embryoid bodies formed are then transferred to 0.1% collage type I (Sigma, C-7661) coated 24-well plate (Corning Incocrporated/Costar 3524, 24 well cell culture Cluster/Flatbottom with Lid/non-pyrogenic polystyrene) in LIF-free stem cell medium containing 100 ng/ml acidic fibroblast growth factor (Sigma, F-3133) and cultured for 3 days.

After 3 days, the medium is replaced with LIF-free stem cell medium containing 20 ng/ml hepatic growth factor (Sigma, H-1404) for 6 days, and then in LIF-free stem cell medium containing 10 ng/ml OSM (Sigma, O-9635), 10 μM Dexamethasone (Sigma, D-6645), 5 μg/ml selenious acid (Aldrich, 22985-7), 50 μg/ml insulin (Invitrogen, I-1882), and 50 μg/ml transferrin (Sigma, T-2036). The differentiated cells are then analyzed for hepatic specific gene expression. The genes analyzed, the annealing temperature for PCR, expected product seizes, and the primer sequences in RTPCR are as follows: transthyretin (TTR) 55° C., 225 bp, 5-CTC ACC ACA GAT GAG AAG, 5-GGC TGA GTC TCT CAA TTC; α-fetoprotein (AFP) 55° C., 173 bp, 5-TCG TAT TCC AAC AGG AGG, 5-AGG CTT TTG CTT CAC CAG; α-1-anti-trypsin (AAT), 55° C., 484 bp, 5-AAT GGA AGA AGC CAT TCG AT, 5-AAG ACT GTA GCT GCT GCA GC; Albumin (ALB), 55° C., 260 bp, 5-GCT ACG GCA CAG TGC TTG, 5-CAG GAT TGC AGA CAG ATA GTC; glucose-6-phophatase (G6P) 55° C., 210 bp, 5-CAG GAC TGG TTC ATC CTT, 5-GTT GCT GTA GTA GTC GGT; tyrosine aminotrnasferase (TAT), 50° C., 206 bp, 5-ACC TTC AAT CCC ATC CGA, 5-TCC CGA CTG GAT AGG GTA G; β-actin 55° C., 200 bp, 5-TTC CTT CTT GGG TAT GGA AT, 5-GAG CAA TGA TCT TGA TCT TC; and SEK1 50° C., 300 bp, 5-TGT ATG GAG CTC ATG TCT ACC; 5-GTC TAT TCT TTC AGG TGC CA.

### Example 4

### Differentiation of Mouse HS Cells into Cells from the Ectodermal Embryonic Layer

### Example 4(a)

### Differentiation into Neuronal Precursor Cells and Functional Postmitotic Nerve Cells

In one embodiment, HS cells were induced to form neuronal precursor cells. Neuroepithelial precursors cells derived from HS cells differentiate into both neurons and glia, and further differentiation leads to expression of a wide variety of neuron-specific genes, and the generation of both excitatory and inhibitory synaptic connections. The expression pattern of position-specific neural markers seen in ES cells demonstrates the presence of a variety of central nervous system (CNS) neuronal cell types. By analogy, it appears that HS cells also give rise to neuronal precursor cells that can efficiently differentiate into functional post-mitotic neurons of diverse CNS structures.

The method of Okabe et al. was used to elicit differentiation of HS cells into a variety of neuronal cells and neurons (Okabe et al., Mech. Dev. 59: 89-102 (1996)).

Materials. The materials were purchased from the following sources: fibronectin, laminin, neurobasal medium, B27 supplement, and superscript II RNase H-reverse transcriptase from Gibco/BRL (Grand Island, N.Y.); bFGF from R&D Systems (Minneapolis, Minn.); insulin, transferrin. selenium chloride, polyornithine, progesterone, putrescine, T3, cytosine arabinoside, anti-MAP2 antibody, anti-NF-M antibody, anti-GABA antibody, and anti-glutamate antibody from Sigma (St. Louis, Mo.); Taq polymerize from Bushranger-Mannheim (Mannheim, Germany); Anti-GFAP antibody from ICN Biomedicals (Costa Mesa, Calif.); anti-keratin 8 antibody from American Type Culture Collection (Rockville, Md.); Vectastain ABC kit from Vector laboratories (Burlingame, Calif.); double staining kit and amino-ethyl carbazole from Zymed Laboratories Inc. (Carlton Court, Calif.) anti-phosphorylated CREB antibody from Upstate Biotechnology Inc. (Lake Placid, N.Y.); BrdU staining kit from Amersham (Arlington Heights; Ill.); fluorescence secondary antibodies from Cappel (Durham, N.C.). Selection of Nestin-Positive Cells. HS cell clumps (or EBs) kept in ES medium suspension culture (see previous examples for medium ingredients) for 4 days were transferred to 15 ml tubes. After the EBs settled, half of the ES culture medium was removed, and 2.5 ml of fresh ES medium was added to the original culture dishes. Dishes were then rinsed with ES medium and added to the same 15 ml tube. EBs were then transferred to tissue new culture dishes. ES medium was changed after 24 h, and ITS medium containing fibronectin (FN), (25 ul of stock/5 ml medium made by carefully layering ES cell-qualified water on 5 mg FN (1 mg/ml) and letting it stand at 4° C. for 30 min), was added. (500 ml ITS medium: DMEM/F12 (1:1) (Gibco 12500-039) 6 g; Insulin (Intergen 4501-01) 2.5 mg dissolved in 0.5 ml sterile H₂O and 5 mcl of 10N NaOH; 30 mcl Selenium Chloride (0.5 mM); 0.775 g glucose; 0.0365 g glutamine; 1.2 g NaHCO₃; and Transferrin (Sigma T-2036) 25 mg; pH 7.5; 5 ml 100×P/S.)

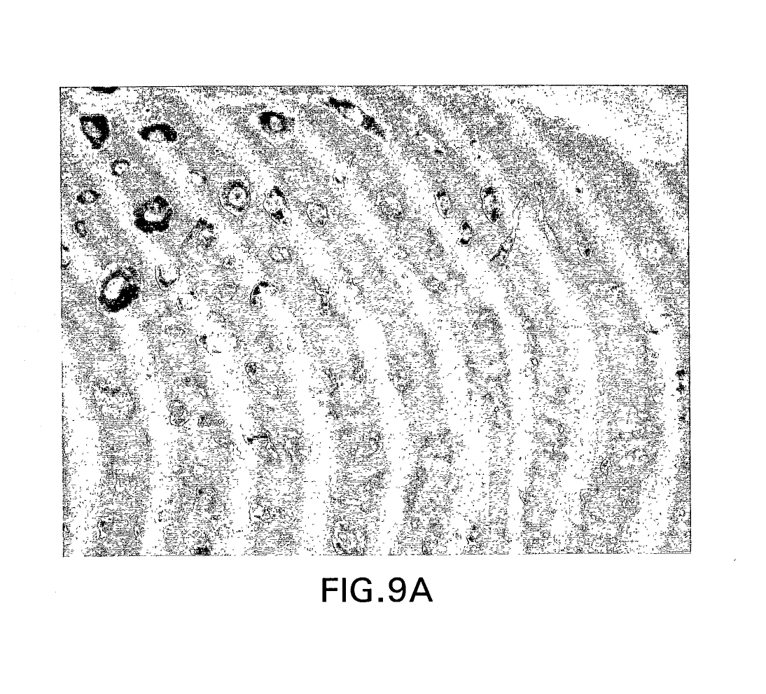

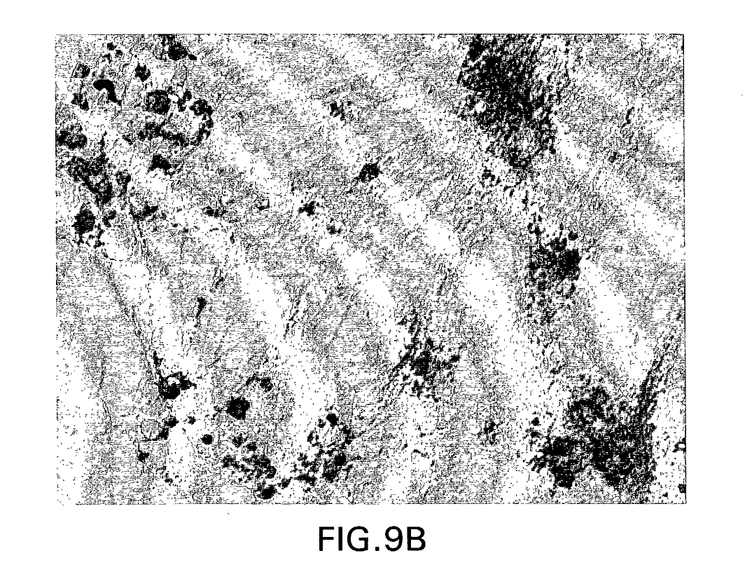

Cells were then fed for 6-10 days in ITS medium containing FN, where medium was changed every two days; FIG. 9A shows nestin-positive cells after 6 days of culturing.

Expansion of Nestin-Positive Cells by bFGF. Cells maintained in ITS/FN medium were washed with PBS twice. 1 ml of trypsin/EDTA solution (0.05% trypsin/0.04% EDTA) was then added to the medium, and cells were maintained at 37° C. for five minutes to cause cell dissociation. 5 ml of ES medium (described in the foregoing example) was then added, and cells were transferred to a 15 ml tube.

EBs were first allowed to settle, and then collected by centrifugation. The cell pellet was resuspended in 5 ml N2 medium containing B27 media supplement (B27 Serum-free supplement 50×, liquid, Gibco 17504-44). (N2 medium 500 ml: f12/DMEM 6 g; glucose 0.775 g; glutamine 0.0365 g; NaHCO₃ 0.845 g; insulin 0.0125 g; 1M putrescine (stock) 50 ul (final 100 uM); 0.5 mM selenite (stock) 30 mcl (final 30 nM; 0.1 mM progesterone (stock) 100 mcl (final 20 nM); adj. PH 7.2; 5 ml 100×P/S.)

Cells were then counted and seeded at a cell density of 5×10⁵ cells/well on 24 well plates (400 mcl N2 medium) or 5-7×10⁶ cells/dish on 6 cm dishes (3 ml N2 medium) on dishes precoated with poly-L-ornithine (15 ug ml⁻¹) and laminin (1 ug ml⁻¹), both obtained from Bector Dickinson Labware, Bedford, Mass. (Precoating protocol: a sterile cover slip (assistent 1001/0012, 12 mm) was inserted into each well (24 well plate); 400 ul poly-L-ornithine (15 ug/ml⁻¹) was then added followed by dilution with PBS from 1000×stock and kept overnight; plates were washed with PBS, and fresh PBS was then added and the plates were placed at 37° C. for 30 minutes; plates were then washed again with PBS and 400 ul of FN (1 ug/ml) was then added followed by dilution with PBS from 1000×stock; plates were placed at 37° C. for at least 2 hs).

N2 culture medium containing 10-20 ng/ml⁻¹ bFGF (R & D Systems, Minneapolis, Minn.) and B27 supplement was then added to the plated cells, and cells were fed on such medium for 6 days. The medium was changed every 2 days. For passage, cells were dissociated by 0.05% trypsin and 0.04% EDTA in PBS, collected by centrifugation, and replated.

Differentiation of Nestin Positive Cells Expanded by bFGF. Differentiation was induced by the removal of bFGF from cell cultures. Cell clumps were allowed to spread for 3-4 days in N2 medium supplemented with laminin (1 mg ml⁻¹) in the presence or absence of cAMP (1 mM), and AA (200 uM), both obtained from Sigma, St. Louis, Mo. Cells were then incubated under differentiation conditions for 6-15 days.

Immunocytochemistry. Cells were fixed with 2% paraformaldehyde in phosphate-buffered saline (PBS; pH 7.4) for 20-30 min permeabilized with 0.2% Triton X-100 in PBS, and treated with 5% normal goat serum. The cells were incubated for 30 min-1 h with the primary antibodies against nestin (1:1000; from Dr. M. Marvin, NIH). Cell may also be incubated with primary anti-bodies against keratin 8 (1:1000), brain fatty acid binding protein (1:1000), MAP2 (1:200), NF-M (1:100), Synapsin I (1:1000), GFAP (1:50), O4, GalC (supernatant of producing cells), GABA (1:1000), and glutamate (1:500). After washing with PBS, cells were processed according to the method for the Vectastain ABC kit.

For double immunofluorescence staining with MAP2 and NF-M, cells can be fixed and permeabilized with Triton X-100 and treated with NGS in a similar manner. The cells can then be incubated with monoclonal anti-MAP2 antibody, followed by fluorescein-labeled anti-mouse IgG, and then fixed again with 2% paraformaldehyde for 30 min. After re-fixation, the cells are incubated with monoclonal anti-NF-M antibody, followed by rhodamine-labeled anti-mouse IgG. The second fixation eliminates the cross-reaction of the rhodamine-conjugated anti-mouse IgO to the anti-MAP2 monoclonal.

For double-label immunocytochemistry with enzyme-linked secondary antibodies, the instructions of the double staining kit (Zymed Laboratories, Inc.) are followed. Staining techniques with anti-phosphorylated CREB antibody (1:1000) are as described by Ginty et al. (1993).

Proliferation Assay. Cells are incubated with BrdU for 8 h at 37° C. After incubation, the cells are immediately fixed and processed according to the instruction of BrdU staining kit. After the color reaction, the cells are incubated with 0.8% hydrogen peroxide and 5% NGS in PBS for 30 mm to inactivate HARP activity. After intense washing, they are processed for either anti-nestin or anti-MAP2 antibody staining to generate a reddish reaction product in the cytoplasm visualized with aminoethyl carbazole.

Cell density is determined by counting the number of cells per field at 200×magnification. Eight fields are analyzed for each sample, and cell densities are calibrated and averaged.

RT-PCR. Total RNA was extracted from each cell preparation by the method of Chomczynski and Sacchi (Chomczynski and Sacchi, Anal Biochem 162: 156-159 (1987)). The total RNA was treated with RNase-free DNase, and cDNA synthesis was performed according to the instructions for superscript II RNase H-reverse transcriptase. PCR reaction was performed in PCR buffer (50 mM KCl, 10 mM Tris-HCI (pH 8.8), 1.5 mM MgCl2, 0-001% (w/v) gelatin) containing 0.2 mM dNTP, 0.3 μM each of forward and reverse primers, and 0.25 U of Taq polymerase. Cycling parameters were denaturing at 9-to C for 30 s, annealing at 55° C. for 30 s, and elongation at 72° C. for 60 s. Cycling times were determined for each primer set to be within the exponential phase of amplification.

Amplification of genomic DNA can be distinguished by the size of products-actin-NMDAR1, NMDAR2D, calbindin D28, GAD65, GABAAa3, AMPA receptor. For other primers, control amplification is done without adding reverse transcriptase to see any amplification of genomic DNA. No amplification of genomic DNA should be observed in control experiments.

Electrophysiology. Cells are recorded at room temperature with 3-6 M Ω patch pipettes containing 130 mM potassium acetate (or 120 CsCl+10 KCl), 10 mM HEPES, 2 mM MgCl₂, 1 mM ATP, 0.1 mM EGTA, 10 mM NaCl, followed by adjusting pH to 7.2 with KOH, and adjusting osmolarity to 300 mosM with sucrose. The recording saline contains 130 mM NaCl, 5 mM KCl, 2 mM CaCl₂, 1 mM MgCl2, 10 mM HEPES, and 10 mM glucose. Osmolarity is adjusted to 320 mosM with sucrose, and pH is adjusted to 7.4 with NaOH. Glutamate (1 mM in the recording saline) is applied by pressure through a micropipette positioned near the recorded cell or near adjacent cells in the field of view, within 100 μm of the recorded cell. Current signals are amplified with an Axopatch amplifier, stored and analyzed on an IBM computer using pClamp-6 software.

Electron Microscopy. Cells in plastic dishes are fixed with 2% paraformaldehyde and 1% glutaraldehyde in PBS for 1 h. Cells are then washed with water, treated with 1% OSO₄, block-stained with uranyl acetate, dehydrated with ethanol and embedded in Araldite resin. Thin-sectioned samples are observed under JEOL 1200 EX electron microscope.

Stimulation of Differentiated Neuronal Cultures. Cells differentiated in neurobasal medium plus B27 and 5% FCS are incubated with the same medium containing 10 μM of either glutamate or NMDA for 10 min. Cells are fixed immediately after stimulation for phospho-CREB staining. Cells are incubated for 50 min after stimulation and RNA is extracted for the analysis of c-fos induction.

### Example 4(b)

### Differentiation of Tyrosine Hydroxylase-Positive Neuronal Cells

HS cells were able to produce Tyrosine Hydroxylase in vitro after several steps of differentiation described as follows. EBs were formed as described in example 1(d) for four days and then plated onto adhesive tissue culture surface in the ES cell medium.

After 24 hours of culture, selection of nestin-positive cells was initiated by replacing the ES cell medium with serum-free Insulin/Transferrin/Selenium/Fibronectin (ITSFn) medium which contains DMEM/F12(1:1), Gibco 11320-033 supplement with Insulin (Sigma I1882) 5 μg/ml, Selenium chloride (Sigma S5261) 30 nM and Fibronectin (Sigma F1141) 5 μg/ml.

After 6-10 days of nestin-selection, cell expansion was initiated. Specifically, the cells were dissociated by 0.05% trypsin/0.04% EDTA, and plated on tissue culture plastic or glass coverslips, which were precoated with 15 μg/ml polyornithine (Sigma, P3655) and 1 μg/ml laminin (Sigma, L2020), at a concentration of 1.5-2×10⁵ cells cm² in N2 medium containing DMEM/F12(1:1), Gibco 11320-033 supplemented with N2 supplement (100×, Gibco 17502-048), 20 μg/ml Insulin, 1 μg/ml of laminin (Sigma, L2020), 10 ng/ml of bFGF (R& D Systems, 233-FB), 500 ng/ml murine N-terminal fragment of SHH (R&D Systems, 461-SH) and 100 ng/ml murine FGF8 isoform b (R&D Systems, 423-F8). The medium was changed every two days.

Differentiation of Tyrosine Hydroxylase positive cells were induced by removal of bFGF from above described medium for expansion with laminin (1 mg/ml ) in the presence or absence of 1 μM cAMP (Sigma, A6885), 200 μM Ascorbic acid (Sigma, A5960). The cells were incubated under differentiation conditions for 6-15 days.

To detect the Tyrosine Hydroxylase positive cells, the induced HS cell culture were rinsed with PBS (phosphate buffered saline, pH 7.4) once and fixed with 4% paraformaldhyde (Electron Microscopy Sciences, 15712) for 30 minutes. The fixed cells were then rinsed 3 times with PBS and treated with methanol for 5 minutes.

The methanol treated cells were again rinsed 3 times with PBS and blocked with block solution (Bottle 1) from Envision+System (Dako, K4010) for 5 minutes.

The excess blocking buffer were tapped off, primary antibody rabbit anti-Tyrosine Hydroxylase (Pel Freez, P40101-0,1:300 in PBS) was applied to the cells and incubated for 60 minutes at room temperature.

The primary antibody stained cells were rinsed 3 times with PBS and the secondary antibody Labelled Polymer (Bottle 2) from Envision+ Systems was applied to cover the cell culture and incubated for 30 minutes at room temperature.

The secondary antibody stained cells were rinsed 3 times with PBS, Liquid DAB+ substrate (Bottle 3) from Envision+ System was added to cover the cell culture and incubated for 5 minutes. Finally the cells were rinsed with PBS 3 times and Tyrosine Hydroxylase positive cells were detected under the microscope (see FIG. 9B showing nestin-positive cells after 6 days of selection.)

1. An isolated homozygous stem cell derived from a method comprising:
(a) isolating a homozygous post-meiosis I diploid germ cell, wherein said germ cell is an oocyte which matured to metaphase II in vivo;
(b) mitotically activating said metaphase II oocyte after an in vitro resting period of between 0-9 hours by preventing the extrusion of the second polar body during oogenesis;
(c) culturing said activated metaphase II oocyte to form a blastocyst-like mass; and,
(d) isolating homozygous stem cells from the inner cell mass of said blastocyst-like mass.

2. The isolated homozygous stem cell of claim 1, wherein the resting period is between 0-1½ hours.

3. The isolated homozygous stem cell of claim 1, wherein the resting period is between 1½-3 hours.

4. The isolated homozygous stem cell of claim 1, wherein the resting period is between 3-5 hours.

5. The isolated homozygous stem cell of claim 1, wherein the resting period is between 5-9 hours.

6. A method of producing homozygous stem cells comprising:
(a) isolating a homozygous post-meiosis I diploid germ cell, wherein said germ cell is an oocyte which matured to metaphase II in vivo;
(b) mitotically activating said metaphase II oocyte after an in vitro resting period of between 0-9 hours by preventing the extrusion of the second polar body during oogenesis;
(c) culturing said activated metaphase II oocyte to form a blastocyst-like mass; and,
(d) isolating homozygous stem cells from the inner cell mass of said blastocyst-like mass.

7. The isolated homozygous stem cell of claim 6, wherein the resting period is between 0-1½ hours.

8. The isolated homozygous stem cell of claim 6, wherein the resting period is between 1½-3 hours.

9. The isolated homozygous stem cell of claim 6, wherein the resting period is between 3-5 hours.

10. The isolated homozygous stem cell of claim 6, wherein the resting period is between 5-9 hours.

11. An isolated homozygous stem cell derived from a method comprising:
(a) producing a mitotically activated homozygous post-meiosis I diploid germ cell by: fusing two oocytes or two spermatids, preventing the extrusion of the second polar body during oogenesis, allowing the extrusion of the second polar body and spontaneous self-replication under appropriate conditions, or transferring two sperm or two haploid egg nuclei into an enucleated oocyte;
(b) culturing said activated homozygous post-meiosis I diploid germ cell to form a blastocyst-like mass;
(c) transplanting said blastocyst-like mass into an animal host to create a stemplasm; and,
(d) culturing said stemplasm in vitro to derive homozygous stem cell lines.

12. An isolated homozygous stem cell derived from the method of claim 11, further comprising screening stem cells that are homozygous by genotyping when a mitotically activated post-meiosis I diploid germ cell is produced by (a) fusing two oocytes or two spermatids, or (b) transferring two sperm or two haploid egg nuclei into an enucleated oocyte.

13. An isolated homozygous stem cell derived from the method of claim 11, wherein said blastocyst-like mass is transplanted under the kidney capsule.

14. An isolated homozygous stem cell derived from the method of claim 11, wherein said blastocyst-like mass mixed with MATRIGEL™ is transplanted in the hind limb of an animal host.

15. A method of producing homozygous stem cells comprising:
(a) producing a mitotically activated homozygous post-meiosis I diploid germ cell by: fusing two oocytes or two spermatids, preventing the extrusion of the second polar body during oogenesis, allowing the extrusion of the second polar body and spontaneous self-replication under appropriate conditions, or transferring two sperm or two haploid egg nuclei into an enucleated oocyte;
(b) culturing said activated homozygous post-meiosis I diploid germ cell to form a blastocyst-like mass;
(c) transplanting said blastocyst-like mass into an animal host to create a stemplasm; and,
(d) culturing said stemplasm in vitro to derived homozygous stem cell lines.

16. The method of claim 15, further comprising screening stem cells that are homozygous by genotyping when a mitotically activated post-meiosis I diploid germ cell is produced by (a) fusing two oocytes or two spermatids, or (b) transferring two sperm or two haploid egg nuclei into an enucleated oocyte.

17. The method of claim 15, wherein said blastocyst-like mass is transplanted under the kidney capsule.

18. The method of claim 15, wherein said blastocyst-like mass mixed with MATRIGEL™ is transplanted in the hind limb of an animal host.

19. A method of making a desired progenitor cell, differentiated cell, group of differentiated cells, or tissue type comprising inducing isolated homozygous stem cells created by the method of claim 1, 11, and 15 to differentiate under suitable conditions.
